(19) United States
(12) Patent Application Publication
(10) Pub. No.: US 2026/0297578 A1
(43) Pub. Date: Oct. 1, 2026

(54) METHODS OF PRESERVING VISION AND TREATING VISION LOSS

(71) Applicant: Massachusetts Eye & Ear Infirmary, Boston, MA (US)

(72) Inventors: Dong Feng Chen, Newtonville, MA (US); Wai Lydia Tai, Boston, MA (US)

(21) Appl. No.: 19/477,876

(22) PCT Filed: Mar. 1, 2024
(86) PCT No.: PCT/US2024/018095

§ 371 Date: Oct. 23, 2025

Related U.S. Application Data
(60) Provisional application No. 63/461,559, filed on Apr. 24, 2023.

(51) Int. Cl. C12N 15/113; A61K 9/00; A61K 31/121; A61K 31/167; A61K 31/4709; A61K 31/506; A61K 31/551; A61K 31/706; A61P 27/02; C12N 15/86

(52) U.S. Cl. CPC C12N 15/113; A61K 9/0019; A61K 31/121; A61K 31/167; A61K 31/4709; A61K 31/506; A61K 31/551; A61K 31/706; A61P 27/02; C12N 15/86; C12N 2310/531; C12N 2750/14143

(57) ABSTRACT

Provided herein are methods of treating optic nerve injury or optic nerve condition, including whole-eye transplantation, often associated with retinal ganglion cell degeneration in a subject (e.g., a human adult), including administering an effective amount of a DNMT3A inhibitory nucleic acid. The DNMT3A inhibitory nucleic acid can be delivered in a vector to a subject intravitreally.

### CLAIM OF PRIORITY

This application claims the benefit of U.S. Provisional Application Ser. No. 63/461,559, filed on Apr. 24, 2023. The entire contents of the foregoing are incorporated herein by reference.

### FEDERALLY SPONSORED RESEARCH OR DEVELOPMENT

This invention was made with Government support under Grant No. EY033882 awarded by the National Institutes of Health. The Government has certain rights in the invention.

### SEQUENCE LISTING

This application contains a Sequence Listing that has been submitted electronically as an XML file named “00633-0385WO1_SL_ST26.XML.” The XML file, created on Feb. 28, 2024, is 3,542 bytes in size. The material in the XML file is hereby incorporated by reference in its entirety.

### TECHNICAL FIELD

The present disclosure relates to methods for treating optic nerve injury or optic nerve degeneration, often associated with retinal ganglion cell degeneration.

### BACKGROUND

Mature neurons in the central nervous system (CNS) of adult mammals regenerate poorly after injury. Eye-to-brain circuits consist of retinal ganglion cells (RGCs) whose axons form the optic nerve and connect to the central target areas. Functional regeneration of the optic nerve cannot be achieved by the activation of a single gene or transcription factor but rather requires modulation of multiple gene expression or programs. The limited regenerative potential of the optic nerve in adult mammals presents a major challenge for restoring vision after optic nerve trauma or disease.

### SUMMARY

Provided herein are methods of restoring vision in a subject with an optic nerve injury or condition comprising administering to the eye of the subject a therapeutically effective amount of an DNMT3a inhibitor. In some embodiments, the DNMT3a inhibitor is a small molecule inhibitor selected from the group consisting of decitabine, SAHA, GSKex1, compound 40, compound 403, Compound 15a, SGI-1027, MC3343, MC3353, BIX-01294, CM-272, CM-579, UV15008, and propiophenone. In some embodiments, the DNMT3a inhibitor is an inhibitory nucleic acid that binds to a portion of DNMT3A. In some embodiments, the inhibitory nucleic acid is antisense RNA, antisense DNA, chimeric antisense oligonucleotides, interference RNA (RNAi), short interfering RNA (siRNA); or a short, hairpin RNA (shRNA). In some embodiments, the administering to the eye of the subject an AAV vector comprises the inhibitory nucleic acid that binds to DNMT3A. In some embodiments, the AAV vector comprises AAV2. In some embodiments, the administration comprises intravitreal injection. In some embodiments, the optic nerve injury or condition is a physical injury. In some embodiments, the physical injury comprises compression injury, ischemia, stroke, trauma, shear force injury, tear injury, and/or surgery. In some embodiments, the optic nerve injury or condition comprises glaucoma, optic neuropathy, ischemic optic neuropathy, optic neuritis, optic nerve atrophy, idiopathic intracranial hypertension, or whole eye transplantation. In some embodiments, the subject is an adult.

Provided herein are nucleic acid compositions having the sequence of SEQ ID NO: 2 or SEQ ID NO: 3, including vectors encoding a nucleic acid having the sequence of SEQ ID NO: 2 or SEQ ID NO: 3.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages will be apparent from the following detailed description and figures, and from the claims.

### DESCRIPTION OF DRAWINGS

FIGS. 1A-N. Dnmt3a deficiency enabled robust axonal outgrowth in retinal explants of mice and human. FIG. 1A: Schematic of RGC axon growth timeline during development (left) and qPCR quantification of Dnmt3a expression in purified RGCs (right) (n=4−5 mice/group); note the inverse correlation of Dnmt3a expression and RGC axon growth capacity from E16 to P10. FIG. 1B: Representative images of retinal sections from mice aged E16. P0, and P10 that were immunolabeled for DNMT3a (red) and an RGC marker RBPMS (green) and counter stained with DAPI (blue). DNMT3a signal was first detected in the ganglion cell layer (GCL) at P0 and became intense and colocalized with RBPMS in P10 retinas. Scale bar: 20 μm. FIG. 1C: Schematic of axon degeneration timeline (left) and qPCR quantification of Dnmt3a expression in purified RGCs (right) from naïve and post-ONC mice (n=3 mice/group). FIGS. 1D-I: Schematics of experiments presented in FIG. 1E and FIG. 1F (FIG. 1D) or FIGS. 1H-J (FIG. 1G), and quantification of axon number (FIG. 1E, FIG. 111) and longest axon length (FIG. 1F, FIG. 1I) in retinal explant cultures derived from adult mice (FIG. 1E, FIG. 1F) and post-mortem human eyes (FIG. 1H, FIG. 1I) that were treated with various doses of a pan DNMT inhibitor decitabine (n=3-6 mice/group). FIG. 1J: Representative images of cultured human retinal explants treated with vehicle (control) or decitabine (bottom) and immunolabeled for β111-tubulin to reveal growing axons. Scale bar: 100 μm. FIGS. 1K-M: Schematic of experiments presented in FIGS. 1L-N (FIG. 1K) and quantification (FIG. 1L, FIG. 1M) of axon number (FIG. 1L) and longest axon length (FIG. 1M) in cultured retinal explants taken from mutant and littermate control (Cre and fl/+) mice (n≥5 mice/group). FIG. 1N: Representative images of cultured retinal explants taken from littermate control (left) and CKO^{Dnmt3a} (right) mice that were immunolabeled for 0-III tubulin. Scale bar: 400 μm. ONBL, outer neuroblastic layer. White arrows denote neurites (*P<0.05, **P<0.01, ***P<0.001, one-way ANOVA; mean s.e.m.) FIGS. 2A-G. Full-length optic nerve regeneration and target reinnervation after ONC in CKO^{Dnmt3+/-} mice. FIG. 2A: Representative images of longitudinal optic nerve sections showing CTB labeled axons in control and CKO^{Dnmt3a+/-} mice at 2- or 16-week post-ONC. Each image is an overlay of 3 consecutive nerve sections. Asterisk indicates the crush site. Scale bar: 100 μm: inset: 20 μm. FIG. 2B: Quantification of axon regeneration by CTB-labeled fluorescent intensity at various distances distal to the crush site at 2 weeks post-ONC (n=5 mice/group). FIG. 2C: Representative images of retinal sections taken from control and CKO^{Dnmt3a+/-} mice at 2 weeks post-ONC that were immunolabeled with anti-RBPMS antibody (green) and counter-stained with DAPI (blue). GCL, ganglion cell layer; scale bar: 20 μm. FIG. 2D: Quantification of RGC survival by counting RBPMS+ cells in retinal sections at 2 weeks post-ONC. White arrows denote RBPMS+ cells (n=3−4 mice/group). FIG. 2E: Schematic of the path of regenerating axons highlighted in purple color, showing their entrance primarily into the contralateral optic chiasm (OC) and optic tract (OT) to innervate the lateral geniculate nucleus (LGN) and Pretectal area. FIG. 2F, FIG. 2G: Representative images of brain sections from CKO^{Dnmt3a+/-} mice at 16 weeks post-crush showing CTB-labeled axons innervating the contralateral (Contra) OC (FIG. 2F, scale bar: 1 mm: inset: 100 μm), OT (FIG. 2G, scale bar: 200 μm), LGN and pretectum (g, scale bar: 200 μm), but not seen in the ipsilateral side (Ipsi). Arrows denote the path of OT, LGN and pretectal areas in Contra and Ipsi brain sections. Insets are circled by orange dash lines. *P<0.05, **P<0.01: for b, multiple unpaired t-tests; for FIG. 2D, unpaired t-test: mean±s.e.m.

FIGS. 3A-J. Visual function recovery following ONC in Dnmt3a deficient mice. FIG. 3A: Schematic of the experiments presented in FIG. 3B-E. FIG. 3B: Quantification of visual acuity assessed by OMR in CKO^{Dnmt3a+/-} and littermate control mice before (baseline, BL) and at 2-16 weeks (wk) after ONC (n≥8mice/group). FIG. 3C: Light perception assessed by the light/dark preference tests in CKO^{Dnmt3a+/-} and littermate control mice before (BL) and at 16 weeks post-ONC: the assay measured the percentage of time spent in the dark chamber (n 5 mice/group). FIG. 3D: VEP N1 wave amplitudes in CKO^{Dnmt3a+/-} and littermate control mice assessed before (BL) and at 16 weeks post-ONC (n≥11 mice/group). FIG. 3E: RGC function measured by pSTR amplitudes in CKO^{Dnmt3a+/-} and littermate control mice before (BL) and at 2-16 weeks after ONC (n≥8 mice/group) (*P<0.05, **P<0.01, ***P<0.001, multiple unpaired t-tests; mean±s.e.m.). FIG. 3F, FIG. 3G: Dimplots showing detection of retinal cell (FIG. 3F) and RGC types (FIG. 3G) in the CKO^{Dmnt3a+/-} and littermate control retinas (n=6 retinas from 6 mice/group) at 2 days after ONC by single nuclei RNA-seq (snRNA-seq). FIG. 3H: Heatmap showing markers unique for each RGC cluster. X-axis indicates the RGC types (cluster annotation), and Y-axis displays the markers used for RGC type identification. FIG. 3I: Single-sample gene set enrichment analysis (ssGSEA) of DEGs from snRNA-seq datasets indicating increased strengths (normalized to controls) in optic nerve morphogenesis, axon genesis, and dendritic extension pathways and decreased strengths in neuronal death and response to axon injury pathways in CKO^{Dnmt3a-/-} RGCs compared to controls. FIG. 3J: Combined GSEA scores in pathways related to neural injury, inflammation, and nerve regeneration among CKO^{Dnmt3a+/-} (blue dotted line) and control (orange dotted line) RGC types. Normalization of the normalized enrichment scores (NES) was carried out using formulas 4-6 described in the Methods. The data revealed across-the-board downregulation of the neuronal injury and inflammation pathways but upregulation of nerve regeneration gene network strength in CKO^{Dnmt3a+/-} RGCs compared to controls. N.D=not detected.

FIGS. 4A-I. Activation of diverse axon regeneration pathways by Dnmt3a deficiency unveiled by multi-omics sequencing data. FIG. 4A: Heatmap of cellular outgoing signals in the retina of CKO^{Dnmt3a+/-} and control mice, predicted by the CellChat output analyses and selected from those showing drastic changes in CKO^{Dnmt3a+/-} and control RGCs: note the downregulation of CSF, GRN, MHC-I and SEMA4 outgoing signals and upregulation of ANGPT and SPP1 outgoing signals in CKO^{Dnmt3a+/-} RGCs. X-axis denotes cell populations: RGC, immune, Müller glia, amacrine, and others (including horizontal cells, bipolar neurons, astrocytes, retinal pigment epithelial cells, rods, and cones). Y-axis denotes signaling pathways (receptor-ligand pairs). FIG. 4B: Chord diagrams representing the intercellular CSF signaling among retinal cell types. The outgoing signal of RGCs was highlighted in green, while the outgoing signals from all other cell types were colored in gray or light pink to simplify the presentation. Arrows indicate the directions of the signals, and the arrow's width represents the relative signal strength. FIG. 4C: Heatmaps of outgoing signals sent among RGC types in control and CKO^{Dnmt3a+/-} mice. Note that CKO^{Dnmt3a+/-} RGCs displayed exclusive transmission of PVR and VISFATIN signals that were absent in control RGCs, while VEGF and IL-4 outgoing signals were observed solely among control RGC types. FIG. 4D: Chord diagrams showing intercellular WNT signaling among RGC types in control and CKO^{Dnmt3a+/-} mice. FIG. 4E: Reduced DNA methylation at the differentially methylated regions (DMR) in CKO^{Dnmt3a+/-} RGCs compared to controls as assessed by whole-genome bisulfite-seq (n=2 mice/group). FIG. 4F: GO analysis of hyper- and hypo-methylated pathways and related genes. FIG. 4G: KEGG pathway enrichment of DMR-related gene pathways, selected from TOP-20. FIG. 4H: KEGG pathway analysis based on the upregulated DEGs from bulk RNA-seq transcriptome profiling of CKO^{Dnmt3a+/-} and control RGCs (n=3 mice/group). Note that the DMR-pathways identified in FIG. 4G and the upregulated signaling events in FIG. 4H are highly correlated—the common pathways found in both are highlighted in orange. FIG. 41: qPCR quantification of selective genes reported to mediate axon regeneration, neural injury and inflammation in CKO^{Dnmt3a+/-} and control RGCs isolated from naïve mice or mice at 2 days after ONC (n≥3 mice/group; *P<0.05, **P<0.01. ***P<0.001, one-way ANOVA; mean s.e.m.).

FIGS. 5A-L. Restoration of vision following ONC in adult mice by therapeutic Dnmt3a knockdown. FIG. 5A: Schematic of the experiments presented in FIG. 5B-FIG. 5M. FIG. 5B: Assessment of visual acuity by OMR assay in adult wildtype mice before (BL) and at 4-12 weeks after ONC; mice received intravitreal injection immediately after ONC of either control AAV.scramble or treatment AAV.shRNA that specifically targeted Dnmt3a. FIG. 5C: Percentage of times spent in the dark chamber by mice in the light/dark preference test. d, pSTR amplitudes. FIG. 5E, FIG. 5F: VEP N1 amplitude (FIG. 5E) and latency (FIG. 5F). FIG. 5G: Assessment of visual acuity by OMR assay in adult Dnmt3a^{fl/+} mice before and at 4-12 weeks after ONC; mice received intravitreal injection immediately after ONC of either control AAV.GFP or treatment AAV.Cre. FIG. 5H: Percentage of time spent in the dark chamber by mice in the light/dark preference test. FIG. 5I: pSTR amplitude. FIG. 5J: Representative pSTR wave forms of (FIG. 5I). FIG. 5K, FIG. 5L: VEP N1 amplitude (FIG. 5K) and latency (FIG. 5L). FIG. 5M, Representative VEP waveforms of (FIG. 5K, FIG. 5L). (n≥3 mice/group: *P<0.05, **P<0.01, ***P<0.001, multiple unpaired t-tests: mean s.e.m.).

FIGS. 6A-N. DNMT expression during development or injury in RGCs of wildtype and mutant mice. FIG. 6A: qPCR quantification of Dnmt1 and Dnmt3b expression in RGCs isolated from E16, P0 and P10 mice. FIG. 61B: Representative image of retinal sections taken from a P10 mouse that was immunolabeled for DNMT3a (red) and counter-stained with DAPI (blue), showing intensive DNMT3a signal in the ganglion cell layer (GCL) and other retinal layers. Scale bar: 20 μm. FIG. 6C: qPCR quantification of Dnmt1 and Dnmt3b expression in RGCs taken at day 2 to 14 after ONC. FIG. 6D: Representative images of retinal sections taken from a Vglut2-Cre:R26-tdTomato mouse that were immunolabeled for RBPMS (green) and counter-stained with DAPI, showing colocalization (orange) of tdTomato red (Cre+) and RBPMS+ cells. INL, inner nuclear layer: ONL, outer nuclear layer. Scale bar: 20 μm: inset: 10 μm. FIG. 6E: Counts of tdTomato red (Cre+) and RBPMS+ cells in Vglut2-Cre:R26-tdTomato mouse retinal sections. FIG. 6F, FIG. 6G: Gene expressions of Dnmt3a in the RGCs (FIG. 6F) and retinas (FIG. 6G) of wildtype, mutant, and corresponding littermate fl/+ control mice. FIG. 6H, FIG. 6I: Gene expression of Dnmt1 in the RGCs (FIG. 6H) and retinas (FIG. 6I) of CKO^{Dnmt1+/-}and littermate control mice. FIG. 6J: Representative images of cultured retinal explants derived from control (left) and CKO^{Dnmt1+/-} mice immunolabeled for β-III tubulin. FIG. 6K, FIG. 6L: qPCR quantification of Dnmt1 (FIG. 6K) and Dnmt3b (FIG. 6L) expression in RGCs of CKO^{Dnmt3a+/-} mice. FIG. 6M, FIG. 6N: Levels of global DNA methylation (5mc %) detected in RGCs and non-RGC retinal cells of control and CKO^{Dn3a+/-} mice (n≥3 mice/group; *P<0.05, **P<0.01, ***P<0.001; for a, c, f, g, one-way ANOVA; for FIG. 6E, FIG. 6H, FIG. 6I, FIG. 6K-FIG. 6N, unpaired t-test; mean±s.e.m.).

FIGS. 7A-E. Reinnervation of brain target by regenerated axons and functional recovery following ONC in CKODnmt3+/− mice. FIG. 7A: Representative image of brain section taken from a control mouse post-ONC showing absence of CTB-labeling in the optic chiasm (OC). Ipsi, ipsilateral; Contra, contralateral. Scale bar: 1 mm, inset: 100 μm. FIG. 7B: Image of brain section taken at the ventral LGN (vLGN) level of a CKODnmt3+/− mouse at 16 weeks post-ONC, showing CTB-labeled axons entering the contralateral vLGN. Scale bar: 200 μm: inset: 100 μm. Insets are circled by orange dash lines. Arrows denote the vLGN. FIG. 7C: VEP N1 latency measured in CKODnmt3a+/− and littermate control mice before (BL) and at 16 weeks post-ONC. Note the prolonged N1 latency in optic nerve-injured CKODnmt3a+/− mice compared to BL while N1 waveform was not detected in control mice, so the N1 latency is expressed as indefinite. (n≥11 mice/group; ***P<0.001, multiple unpaired t-tests; mean+s.e.m.). FIG. 7D, FIG. 7E: Representative waveforms of the VEP (FIG. 7D) and pSTR (FIG. 7E) taken from CKODnmt3a+/− and littermate control mice before (BL) and at 16 weeks post ONC.

FIGS. 8A-D. Alterations in the RGC transcriptomes but not type distributions. FIG. 8A: Dimplot showing RGC clustering in snRNA-seq dataset merged from control and CKO^{Dnmt3a+/-} mice and identifying 35 clusters with 42 out of 45 RGC types (annotation showing multiple RGC types, e.g. W3D2|W3D3, in some clusters). FIG. 8B: Feature plot showing expression of Rbpms, a pan-RGC marker, in the entire retinal cell clusters and RGC subsets in merged snRNA-seq datasets of control and CKO^{Dnmt3a+/-} mice. FIG. 8C: Barplot showing RGC types frequency with the total number of RGCs to be 3791 for control and 3319 for CKO^{Dnmt3+/-}, respectively. FIG. 8D: Scatterplot visualization of relative strength of combined Gene set enrichment analysis (GSEA) pathway scores related to neuronal injury (n=4 GSEA pathways) and nerve regeneration (n=2 GSEA pathways) between CKO^{Dnmt3+/-} and control RGC types. The difference was calculated by subtraction of the normalized average expression in CKO^{Dnmt3+/-} RGCs from the Controls. Each colored dot represents a RGC type noted in the dimplot (FIG. 8A).

FIGS. 9A-B. Wide-scale shifts of transcriptome profiles in RGC types. FIG. 9A, FIG. 9B: Heatmaps of Gene ontology biological processes (GOBP) analysis for snRNA-seq DEGs in individual RGC clusters indicating increased strengths in optic nerve morphogenesis (FIG. 9A), axonogenesis, dendritic extension, and axon development (FIG. 9B), but decreased strengths in neuronal death and response to axon injury (FIG. 9B). NES, normalized enrichment scores.

FIGS. 10A-B. Comparison of Pten and Socs3 expression in various RGC types of CKO^{Dnmt3+/-} and control mice. FIG. 10A: Heatmaps of Gene ontology biological processes (GOBP) analysis for snRNA-seq DEGs in individual RGC clusters indicating reduced acute inflammatory response, and inflammatory cytokine production in CKO^{Dnmt3+/-} RGCs compared to controls. NES, normalized enrichment scores. FIG. 10B: Dotplot visualization of Pten (top) and Socs3 (bottom) expression in individual RGC clusters detected by snRNA-seq analysis of CKO^{Dnmt3+/-} and control RGCs. Red stars denote RGC types with significantly downregulated expressions of Pten or Socs3 in the CKO^{Dnmt3+/-} compared to the control (P<0.05).

FIGS. 11A-B. CellChat predictions of outgoing signals from different retinal cell types. FIG. 11A, FIG. 11B: CellChat heatmap representing all predicted outgoing signaling patterns from retinal cells of control (FIG. 11A) and CKO^{Dnmt3+/-} (FIG. 11B) mice. The cell types analyzed include RGC, immune cells (including microglia/macrophages, T helpers and T cells), Müller glia, amacrine, and other (including horizontal cells, bipolar neurons, astrocytes, retinal pigment epithelium, rods, and cones). The top-colored bar plot demonstrates the total signaling strength of a cell group by summarizing all signaling pathways shown on the heatmap. X-axis denotes the cell populations, and Y-axis denotes the pathways in an order of signaling strength/contribution.

FIGS. 12 A-B. CellChat predictions of incoming and outgoing signals sent within RGC types. FIG. 12A, FIG. 12B: CellChat heatmap representing all predicted incoming and outgoing signaling patterns among RGC types of control (FIG. 12A) and CKODnmt3+/− (FIG. 12B) mice. Colored bars shown on top of the heatmap illustrate the total signaling strength of a cell group by summarizing all signaling pathways. X-axis denotes the RGC types, and Y-axis denotes the pathways in an order of signaling strength/contribution.

FIGS. 13 A-D. DMR demethylation and upregulation of axon regeneration pathways by Dnmt3a deficiency. FIG. 13A: The total methylation level of CG in the RGCs of control and CKODnmt3a+/− mice at 2-day post-crush (n=2 mice/group). FIG. 13B: Circos plot of CG methylation level and the difference between control and CKODnmt3a+/− RGCs. From outside to inside, each ring represents: 1. methylation level for the CKODnmt3a+/− group, 2. methylation level difference between control and CKODnmt3a+/−(heatmap) 3. methylation level for the control group. The chromosomes were divided into bins where methylation level of each bin is calculated as the number of reads with methylation/(number of reads with methylation+number of reads without methylation). FIG. 13C: Cluster heatmap for CG methylation level at the differentially methylated regions (DMRs). FIG. 13D: Principal component analysis of bulk RNA-seq data (n=3 mice/group), demonstrating the variability of the samples studied. FIG. 13E: Volcano plot of DEGs between CKODnmt3+/− and control RGCs by bulk RNA-seq. NS, non-significant; Log 2 FC, DEGs with log 2 fold change>1 and p-value>0.05; p-value, DEGs with p-value<0.05 and log 2 fold change<1.

FIGS. 14A-E. Knockdown of Dnmt3a by AAV-mediated gene delivery. FIG. 14A: Kyoto encyclopedia of genes and genomes (KEGG) pathways enriched by downregulated genes in bulk RNA-seq. FIG. 14B: Gene set enrichment analysis (GSEA) Hallmark pathways and (FIG. 14C) Gene ontology (GO) Terms enriched by upregulated (labeled in yellow) and downregulated (labeled in purple) genes in bulk RNA-seq. (FIG. 14D) qPCR quantification of Dnmt3a expression in RGCs and non-RGC retinal cells in adult wildtype mice at 14 days after receiving intravitreal injection of AAV.Scramble or AAV.shRNA that specifically targets Dnmt3a. (FIG. 14E) qPCR quantification of Dnmt3a expression in RGCs and non-RGC retinal cells in adult Dnmt3a fl/+ mice at 14 days after receiving intravitreal injection of AAV.GPF or AAV.Cre (n≥3 mice/group: *P<0.05, unpaired t-test; mean s.e.m.).

### DETAILED DESCRIPTION

Embryonic RGCs possess the innate ability to regenerate optic nerve fibers and readily grow their axons even when presented with a hostile or adult brain environment (growth-inhibitory)^{5,6}. However, this ability of RGCs is lost perinatally⁴⁻⁷ a time coinciding with dynamic changes in epigenetic factors during the RGC maturation⁸. A recent study demonstrated promising results in reprogramming the epigenomes of adult RGCs through the ectopic expression of three transcription factors used to generate stem cells—Oct4, Sox2, and Klf4 (Yamanaka factors)⁹. This resulted in partial restoration of youthful DNA methylation patterns and moderate axon regeneration following crush injury that did not reach the brain or the central vision targets which is an essential step for functional restoration. To date, however, no known strategy fully restores optic nerve regeneration with visual recovery following nerve injury in adult mammals. As shown herein, restoration of vision after traumatic injury of the optic nerve is achieved via suppression of Dnmt3a, which can overcome the inability of adult RGCs to regenerate axons. Inhibition of DNMT3a-dependent DNA methylation reactivates an intrinsic axon growth program that is lost in adults, promotes RGC survival, and enables axonal regeneration through the optic nerve to central targets. As regenerating axons reach the brain, spatial vision is recovered. Provided herein are materials and methods for inducing the regeneration and repair of the optic nerve to treat an optic nerve injury or condition, including whole-eye transplant, by administering inhibitors of DNMT3A.

### Dnmt3a

The DNMT3A gene encodes the enzyme DNA methyltransferase 3 alpha. This enzyme is involved in DNA methylation, or the addition of methyl groups to DNA molecules, and in particular to cytosine nucleotides at CpG sites. This process is called de novo DNA methylation. The DNMT3A protein includes the Pro-Trp-Trp-Pro (PWWP) domain, the ATRX-DNMT3-DNMT3L (ADD) domain, and the catalytic methyltransferase domain. DNMT3A is widely expressed among mammals and highly homologous between human and murine homologues.

As shown herein, DNMT3a is a potent inhibitor of axon regeneration in mouse and human retinal explants. DNMT3a is pivotal for postnatal onset of optic nerve regenerative failure, and suppression of this single gene results in reprogramming of RGC transcriptomic landscape to enable unprecedented level of optic nerve regeneration and reversal of vision loss in adult mice. Applicants have found that suppressing the DNMT3a expression or function in both mouse and human retinal explant cultures or via adenoviral vector-mediated shRNA that targets the common RNA sequence of mouse and human Dnmnt3a in RGCs unlocks the epigenetic switch for optic nerve regeneration and presents a therapeutic avenue for effectively reversing vision loss resulted from optic nerve trauma or diseases.

In some embodiments, methods of restoring vision include administering to the subject a DNMT3A inhibitor. In some embodiments the DNMT3A inhibitor is a small molecule compound selected from the group consisting of: Decitabine, SAHA, GSKex1, compound 40, compound 40_3, Compound 15a, SGI-1027, MC3343, MC3353. BIX-01294, CM-272, CM-579, UV15008, and propiophenone. See Wong KK, Lawrie CH, Green TM. Oncogenic Roles and Inhibitors of DNMT1, DNMT3A, and DNMT3B in Acute Myeloid Leukaemia. Biomark Insights. 2019 May 8; 14:1177271919846454. Doi: 10.1177/1177271919846454; Hu, C., Liu, X., Zeng, Y. et al. DNA methyltransferase inhibitors combination therapy for the treatment of solid tumor: mechanism and clinical application. Clin Epigenet 13, 166 (2021). doi.org/10.1186/s13148-021-01154-x; Zhang Z, Wang G, Li Y, Lei D, Xiang J, Ouyang L, Wang Y, Yang J. Recent progress in DNA methyltransferase inhibitors as anticancer agents. Front Pharmacol. 2022 Dec. 16:13:1072651. doi: 10.3389/fphar.2022.1072651. In some embodiments, the DNMT3A inhibitor is an inhibitory nucleic acid that targets DNMT3A. Exemplary human DNMT3A sequences are provided in GenBank; see Table 1.

TABLE 1
DNMT3A, exemplary human sequences

| | | Tran- | |
|---|---|---|---|
| | Transcript- | script- | |
| Transcript- | protein- | protein- | |
| accession | accession | isoform | Transcript-name |
| NM_022552.5 | NP_072046.2 | isoform a | transcript variant 3 |
| NM_175629.2 | NP_783328.1 | isoform a | transcript variant 1 |
| NM_153759.3 | NP_715640.2 | isoform b | transcript variant 2 |
| NM_001320893.1 | NP_001307822.1 | isoform d | transcript variant 6 |
| NM_001375819.1 | NP_001362748.1 | isoform e | transcript variant 8 |
| NM_175630.1 | NP_783329.1 | isoform c | transcript variant 4 |
| NM_001320892.2 | NP_001307821.1 | isoform c | transcript variant 5 |

### Inhibitory Nucleic Acids

Inhibitory nucleic acids useful in the present methods and compositions include antisense oligonucleotides, ribozymes, extemal guide sequence (EGS) oligonucleotides, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of DNMT3A and suppress its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); or a short, hairpin RNA (shRNA), or combinations thereof. See, e.g., WO 2010040112.

The DNMT3a inhibitor includes an inhibitory nucleic acid capable of binding to mammalian DNMT3A (e.g., mice, rats, sheep, cows, goats, dogs, cats, pigs, guinea pigs). The DNMT3A inhibitor includes an inhibitory nucleic acid capable of binding to human DNMT3A. In some embodiments, the inhibitory nucleic acid binding to DNMT3A comprises a nucleic acid of at least 80%, at least 90%, at least 95%, at least 97%, at least 99% sequence identity to SEQ ID NO: 2 or SEQ ID NO: 3. In some embodiments, the inhibitory nucleic acid binding to DNMT3A, binds a portion of a human sequence listed in Table 1. DNMT3a is evolutionarily conservative, so it shares a high homology across species. In some embodiments, the DNMT3a inhibitor does not include SEQ ID NO: 2 or SEQ ID NO: 3.

The inhibitory nucleic acids useful in the present methods are sufficiently complementary to a human DNMT3A RNA, e.g., mRNA, to hybridize sufficiently well and with sufficient specificity, to give the desired effect. A 100% complementarity is not required. Routine methods can be used to design an inhibitory nucleic acid that binds to the DNMT3A sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example. “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides). In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 11, 12, 13, 14, 15, 16, 17, 18, 19, 20, 21, 22, 23, 24, 25, 26, 27, 28, 29, 30, 31, 32, 33, 34, 35, 36, 37, 38, 39.40, 41.42, 43, 44, 45, 46, 47, 48, 49, or 50 nucleotides in length (complementary portions refers to those portions of the inhibitory nucleic acids that are complementary to the target sequence).

In general, the inhibitory nucleic acids useful in the methods described herein have complementarity to a portion of the DNMT3A mRNA sufficient to inhibit translation of the DNMT3A protein. Percent complementarity of an inhibitory nucleic acid with a region of the DNMT3A can be determined routinely using basic local alignment search tools (BLAST programs) (Altschul et al., J. Mol. Biol., 1990, 215, 403-410; Zhang and Madden. Genome Res., 1997, 7, 649-656). Inhibitory nucleic acids that hybridize to an RNA can be identified through routine experimentation. In general, the inhibitory nucleic acids must retain specificity for DNMT3A, i.e., must not directly bind to, or directly significantly affect expression levels of, transcripts other than the intended target.

In some embodiments, a nucleic acid sequence that is complementary to DNMT3A RNA can be an interfering RNA, including but not limited to a small interfering RNA (“siRNA”) or a small hairpin RNA (“shRNA”). Methods for constructing interfering RNAs are well known in the art. For example, the interfering RNA can be a polynucleotide with a duplex, asymmetric duplex, hairpin, or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to DNMT3A sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in the DNMT3A or a portion thereof and the sense region having nucleotide sequence corresponding to the DNMT3A sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553. (2002); Lee et al, Nature Biotechnol., 20, 500-505, (2002); Mivagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc NatlAcadSci USA 99:6047-6052, (2002).

The DNMT3A RNA cleavage reaction guided by siRNAs is highly sequence specific. In general, siRNA containing a nucleotide sequence identical to a portion of DNMT3A sequence is preferred for inhibition. However, 100% sequence identity between the siRNA and the DNTM3a gene is not required to practice the present methods. Thus, the compositions and methods have the advantage of being able to tolerate sequence variations that might be expected due to genetic mutation, strain polymorphism, or evolutionary divergence. For example, siRNA sequences with insertions, deletions, and single point mutations relative to the DNMT3A sequence can be effective for inhibition. Alternatively, siRNA sequences with nucleotide analog substitutions or insertions can be effective for inhibition. In general, the siRNAs must retain specificity for DNMT3A, i.e., must not directly bind to, or directly significantly affect expression levels of, transcripts other than the DNMT3A.

### Vectors

The present methods can include delivery of the inhibitory nucleic acids naked (e.g., as synthetic nucleic acids) or in an expression vector. Exemplary viral vectors for use in the present methods and compositions include recombinant retroviruses, adenovirus, adeno-associated virus, alphavirus, and lentivirus.

A preferred viral vector system useful for delivery of nucleic acids in the present methods is the adeno-associated virus (AAV). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988): Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. In some embodiments, the AAV is AAV1, AAV2, AAV4, AAV5, AAV6. AV6.2. AAV8, AAV9, rh.10, rh.39, rh.43 or CSp3, or a pseudotyped AAV. Non-limiting examples of derivatives and pseudotypes include AAVrh.10, rAAV2/1, rAAV2/5, rAAV2/8, rAAV2/9, AAV2-AAV3 hybrid, AAVhu.14, AAV3a/3b, AAVrh32.33, AAV-HSC15, AAV-HSC17, AAVhu.37, AAVrh.8, CHt-P6, AAV2.5, AAV6.2, AAV2i8. AAV-HSC15/17, AAVM41, AAV9.45, AAV6(Y445F/Y731F), AAV2.5T, AAV-HAEl/2, AAV clone 32/83, AAVShH10, AAV2 (Y->F), AAV8 (Y733F), AAV2.15, AAV2.4, AAVM41, and AAVr3.45. AAV serotypes and derivatives/pseudotypes, and methods of producing such are known in the art (see, e.g., Mol Ther. 2012 April; 20(4):699-708). In some embodiments, the rAAV particle is a pseudotyped rAAV particle, which comprises (a) an rAAV vector comprising ITRs from one serotype (e.g., AAV2, AAV3) and (b) a capsid comprised of capsid proteins derived from another serotype (e.g., AAV1, AAV2, AAV3, AAV4. AAV5. AAV6. AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

In some embodiments, the AAV is chosen based on its natural tropism (see, e.g., Table 2). (Table 2 adapted from Table 1 of Komeyenkov and Zamyatnin, Jr., Pharmaceutics 2021, 13(5), 750).

TABLE 2
Characterization of AAV natural serotypes.

| Serotype | Natural Tropism |
|---|---|
| AAV1 | Muscle, CNS, heart, liver, lungs |
| AAV2 | Heart, CNS, liver, lungs, retina |
| AAV3 | Liver |
| AAV4 | Retina, lungs, kidney |
| AAV5 | Retina, CNS, liver |
| AAV6 | Heart, liver, muscle, retina |
| AAV7 | Liver |
| AAV8 | Muscle, heart, CNS, liver |
| AAV9 | Heart, CNS, liver |
| AAV10 | Muscle, myoblast tissue |
| AAV11 | Muscle, myoblast tissue |
| AAV12 | Salivary glands, muscle |

In some embodiments the method includes an AAV2 (adeno-associated virus) vector comprising a nucleotide sequence encoding an inhibitory nucleic acid capable of binding to DNMT3A. In some embodiments the method includes an AAV vector comprising the inhibitory nucleic acid capable of binding to DNMT3A. In some embodiments the vector also comprises a U6 promoter.

In some embodiments, the AAV also has one or more additional mutations that increase delivery to the target tissue, e.g., the CNS, or that reduce off-tissue targeting, e.g., mutations that decrease liver delivery when CNS, heart, or muscle delivery is intended (e.g., as described in Pulicherla et al. (2011) Mol Ther 19:1070-1078): or the addition of other targeting peptides, e.g., as described in Chen et al. (2008) Nat Med 15:1215-1218 or Xu et al., (2005) Virology 341:203-214 or U.S. Pat. Nos. 9,102,949; 9,585,971; and US20170166926. See also Gray and Samulski (2011) “Vector design and considerations for CNS applications,” in Gene Vector Design and Application to Treat Nervous System Disorders ed. Glorioso J., editor. (Washington, DC: Society for Neuroscience:) 1-9, available at sfn.org/~/media/SfN/Documents/Short %20Courses/20110%20Short %20Course %20I/2011_SC1_Gray.ashx.

The AAV should also include a promoter to drive expression of the inhibitory nucleic acid. In some embodiments, expression is driven by a ubiquitous promoter, such as cytomegalovirus (CMV); a hybrid CMV enhance/chicken R-actin (CBA) promoter; a promoter comprising the CMV early enhancer element, the first exon and first intron of the chicken β-actin gene, and the splice acceptor of the rabbit β-globin gene (commonly call the “CAG promoter”); beta glucuronidase (GUSB); ubiquitin; UBC, Rous sarcoma virus (RSV) promoter; or a 1.6-kb hybrid promoter composed of a CMV immediate-early enhancer and CBAintron 1/exon 1 (commonly called the CAGGS promoter; Niwa et al. Gene, 108:193-199 (1991)). Alternatively, a promoter for a gene that is specifically expressed in retinal ganglion cells can be used, e.g., CMV, Thy-1, Mash5, Vglut2, mSncg, CBA, RPE65, and rhodopsin promoters. Modifications of these sequences may be possible or desirable in certain applications, and such modifications are within the scope of this disclosure. The woodchuck hepatitis virus posttranscriptional response element (WPRE) can also be used.

In some embodiments, the AAV vector comprises at least, in order from 5′ to 3′, a first adeno-associated virus (AAV) inverted terminal repeat (ITR) sequence, a promoter operably linked to a nucleotide sequence encoding an inhibitory nucleic acid targeting DNMT3A, a polyadenylation signal, and a second AAV inverted terminal repeat (ITR) sequence.

### Methods of Treatment

As discussed in the examples, the suppression of DNMT3A restored visual function after optic nerve injury. Administration of a therapeutically effective amount of a DNMT3A inhibitor as described herein for the treatment of optic nerve injury can overcome the inability of adult RGCs to regenerate axons and result in RGC and/or axonal regeneration. Generally, the methods include administering a therapeutically effective amount of the DNMT3A inhibitory nucleic acid as described herein, to a subject in need of, or who has been determined to be in need of, such treatment. Therefore, the methods here can include methods of regenerating an optic nerve in a mammal, methods of growing retinal ganglion cell axons, methods of reinnervating a damaged optic nerve, methods of treating damaged optic nerve neurons, methods of treating a neurodegenerative disease in a subject, and methods of recovering retinal ganglion cell function.

The methods described herein include methods for the treatment of optic nerve injury, RGC degeneration, and/or optic nerve disease or condition. In some embodiments, the injury is optic nerve crush. In some embodiments, the injury is optic nerve puncture. In some embodiments, the injury is a physical injury (e.g., compression injury, e.g., swelling, ischemia, stroke, trauma, shear force injury, tear injury, and/or surgery, e.g., surgical optic nerve transection) to the optic nerve. In some embodiments, RGC degeneration is caused by Parkinson's disease, Alzheimer's disease, or a demyelinating disease. In some embodiments, the optic nerve disease or condition is glaucoma, optic neuropathy, ischemic optic neuropathy, optic neuritis, optic nerve atrophy, idiopathic intracranial hypertension, or whole eye transplantation.

In some embodiments, the inhibitory nucleic acid can be used without a vector in the methods described herein to treat vision loss and/or restore vision. In some embodiments, the DNTM3A inhibitory nucleic acid (e.g., described herein) can be used in combination with the AAV vector (e.g., AAV2) in the methods described herein to reinnervate a damaged optic nerve and restore vision.

In some embodiments the administration of the inhibitory nucleic acid includes intravitreal injection, suprachoroidal delivery, port delivery, or subretinal delivery. In some embodiment, the subject is mammal (e.g., human) where the mammal is a juvenile or an adult.

### EXAMPLES

The invention is further described in the following examples, which do not limit the scope of the invention described in the claims.

### Methods

The methods described here were used in the Examples below.

### Mice

Vglut2-ires-Cre knock-in (Vglut2-Cre) mice were generated in Bradford B Lowell's laboratory (Harvard University) (Varadarajan et al., 2022) and Dnmt3a floxed (Dnmt3a^{flox}) mice were obtained from Guoping Fan's laboratory (University of California, Los Angeles) (Williams et al., 2020). C57BL/6J wildtype (000664) and B6.Cg-Gt(ROSA)26Sortm9(CAG-tdTomato)Hze/J (007909) mice were purchased from the Jackson Laboratory. Both sexes of animals were used in the experiments. The control group for experiments with Cre/lox mice included Vglut2-Cre, floxed, and littermate wildtype mice. All animal procedures were approved by the Institutional Animal Care and Use Committees (IACUCs) at the Schepens Eye Research Institute (SERI), Mass Eye and Ear. Animals were bred and housed under 12-h light/dark cycles with food and water ad libitum in the SERI animal facility.

### RGC Isolation

As described previously (Kristensen et al., 2009), mouse retinas were dissected in Neurobasal-A medium (Gibco, 10888-022) on ice and dissociated into single-cell suspensions in papain supplemented with deoxyribonuclease I and Ovomucoid protease inhibitor by manual trituration according to the manufacturer's protocol (Papain Dissociation System. Worthington, Lk003150). The resulting cells were spun down and resuspended in autoMACS Rinsing Solution (Miltenyi Biotec, 130-091-222) followed by incubation with micro-magnetic beads conjugated Thy1.2 antibody (Miltenyi Biotec, 130-121-278) for 20 min at 4° C. Cells were then loaded into the MS Column (Miltenyi Biotec, 130-042-201) held by a magnetic separator (Miltenyi Biotec. 130-042-108) that allowed separation of magnetic beads-labeled cells from the rest by rinsing the column. After 3 washings, the column was removed from the separator and the labeled cells were gently flushed into a collection tube with 1 ml of the rinsing solution. The collected cells were spun down and processed for RNA or DNA analysis.

### Total Nucleic Acids Extraction

Total RNA and DNA were extracted using the Zymo Quick-RNA MicroPrep Kit (Zymo Research. R1051) and Quick-DNA MiniPrep Kit (Zymo Research, D3024) respectively. The quantity and quality of both nucleic acids were measured using a NanoDrop 2000 Spectrophotometer (Thermo Fisher Scientific). RNA was then frozen and sent to Novogene for ultra-low-input bulk RNA sequencing or converted into cDNA with the PrimeScript RT Master Mix (Takara Bio, RR036A) for quantification of mRNA level using qPCR (Chen et al.,1998). DNA was frozen and sent to Novogene for whole genome bisulfite sequencing (WGBS) or processed for assessment of global methylation level.

### Global DNA Methylation Assay

Genome-wide DNA methylation levels were determined using the 5-mC DNA ELISA Kit (Zymo Research, D5325) per the manufacturer's instruction. Absorbance was recorded at 405 nm with a microplate reader (Agilent, BioTek Synergy H1 Multimode Reader). The percentage of methylated cytosines (% 5-mC) in the total DNA content was determined by calculating the levels of 5-mC using a standard curve generated with the kit controls.

### Human Eye Globes

The Lions Eye Bank in Florida provided human cadaver eye globes for the experiments. These eye globes were obtained from donors between the ages of 68 and 92. All experiments involving human tissue were conducted according to the principles of the Declaration of Helsinki.

### Retinal Explant Culture

Retinas dissected out from post-mortem human eyes were cut at both central and peripheral areas with a biopsy punch (Acuderm Inc, P2525) in ice-cold Neurobasal-A medium, producing small explants in 2.5 mm diameter. Retinas from adult mice were cut into 4-6 equally sized pieces. With the RGC layer facing down, retinal explants were placed onto tissue culture inserts (6-well plate format, Greiner Bio-One, 657641) that were pre-coated with Matrigel matrix (Corning, 354230). Inserts were then placed into culture plate wells containing Neurobasal-A medium supplemented with 25 μM L-Glutamic acid (Sigma-Aldrich, G8415). 2 mM Glutamax (Gibco, 35050061), 1×penicillin/streptomycin (Thermo Scientific, 15140122), 1×B-27a (Gibco, 17504-044), 5 μg/ml insulin (Sigma-Aldrich, 19278), 50 ng/ml BDNF (PeproTech, 450-02), 50 ng/ml CNTF (PeproTech, 450-13), and 1×forskolin (Sigma-Aldrich, F6886). Explants were cultured at 37° C. with 5% CO2 for 7 days and then processed for immunohistochemical analysis.

### Quantification of Neurite Outgrowth in Explant Cultures

After 7 days of incubation, retinal explants on inserts were fixed with 4% PFA for 2 h and blocked with Mojito buffer (10% NGS, 3% NDS, 1% BSA, 0.5% Tween-20, 0.5% Triton X-100 and 0.1% sodium citrate buffer in PBS) for 2 h at room temperature. To stain for neurites, explants were incubated with mouse anti-p Tubulin III (TUJI) antibody (1:500, Novus Biologicals, NB600-1018) overnight at 4° C. followed by Alexa Fluor 594-conjugated secondary antibody (1:500. Jackson ImmunoResearch) for 2 h at room temperature. All antibodies were diluted in a staining buffer consisting of 0.5% Triton X-100, 0.1% Tween-20 and 5% BSA in PBS. Between changes of solution, explants were washed 3 times, 10 min each with PBS. After mounting onto Superfrost Plus Slides (VWR, 48311-703) with Fluoromount-G (SouthemBiotech. 0100-20), explants were imaged with a Leica fluorescence microscope (Leica Microsystems, DMi8). The neurite number and length of each retinal explants were quantified using the built-in software LAS X of the microscope (Leica Microsystems, Leica Application Suite X) by individuals blinded to the experimental groups.

### Optic Nerve Crush Injury

Mice were anesthetized by inhalation of 3-4% isoflurane for induction and 1-3% isoflurane for maintenance. Detailed surgical procedure of unilateral optic nerve crush (ONC) has been described previously (Chen et al., 1997; Chen et al., 1995). In brief, a small incision was made in the temporal conjunctiva of the mouse eye, and the optic nerve was exposed and crushed with a Dumont #5 jeweler's forceps (FST) for about 5 sec. approximately 0.5 mm behind the eyeball. Proparacaine and antibiotic ointment were applied to the ocular surface before and after the crush, respectively. Buprenorphine SR or Ethiqa X were given subcutaneously as a postoperative analgesic.

### Immunohistochemistry of Cryosections

Mouse eye and brain were dissected out after transcardiac perfusion with 4% PFA in PBS. Tissues were post-fixed in 4% PFA overnight and then cryoprotected in 30% sucrose overnight at 4° C. For cryosections of the eye, cornea and lens were removed from the eye before being embedded in OCT compound (VWR, 25608-930) and frozen. The frozen tissue block was then sliced into 16 μm serial cross sections, collected on Superfrost Plus Slides and stored at −20° C. until processed. Frozen sections were blocked with BSA (5%) and Triton X-100 (1%) in TBS for 1 h at room temperature, followed by incubation with the primary antibodies overnight at 4° C. and then the proper secondary antibodies (1:500, Alexa Fluor 488, 594, 647 conjugated; Jackson ImmunoResearch) for 2 h at room temperature in the same blocking solution. Between changes of antibody incubation, all sections were washed 3 times, 5 min each. For staining of cholera toxin subunit B (CTB), after primary antibody, sections were incubated with biotin anti-goat (1:250, Vector Laboratories, BA-9500) for 2 h and Alexa Fluor 594-conjugated streptavidin (1:500, Thermo Scientific, S32356) for 1 h at room temperature. Primary antibodies used were goat anti-CTB (1:4000, List Biological Laboratories. 703), guinea pig anti-RBPMS (1:200, PhosphoSolutions, 1830-RBPMS), and mouse anti-DNMT3a (1:100, Novus Biologicals, NB120-13888SS). Staining for anti-DNMT3a required 2N HCl treatment for 30 min at 37° C. followed by neutralization with 0.1M Trish-HCl (pH 8.3) for 10 min at room temperature before the blocking.

For cryosections of the brain, OCT-embedded tissue block was cut into 40 μm serial cross sections, collected in cryoprotectant solution and stored at −20° C. until processed. The floating brain sections were blocked with Background Buster (Newcorner Supply, NB306-50) for 30 min at room temperature followed by a previously published staining protocol with slight modification. Briefly, after 4-day primary incubation with goat anti-CTB (1:4000) at 4° C. floating sections were stained with biotin anti-goat for 2 h (1:200) and Alexa Fluor 594-conjugated streptavidin (1:1000) for 2 h at room temperature.

Immunostained samples were mounted with Fluoromount-G and imaged using Leica DMi8 fluorescence microscope and Leica SP8 confocal microscope (Leica Microsystems) with 20× and 40× lens, and Olympus FluoView FV3000 confocal laser scanning microscope (Olympus Life Science) with 4× lens. Tile scans of 3-4 consecutive retinal and longitudinal optic nerve sections per animal were imaged and used for RGC and axon quantification where data from the scans were averaged for individuals. Projection of Brain Z-scans was carried out using the ImageJ (1.53t) Fiji (2.13.1) with the ‘Average intensity’ projection option. Quantification of regenerated axons was adapted from protocols described previously (Goldberg et al., 2002). In brief, within the microscope's built-in software LAS X, the fluorescence intensities of CTB-positive axons at different distances post the crush site were measured as the integrated density of the Region of Interest (ROI) subtracting the background. For quantification of RGC density in retinal sections, retinal sections containing the optic nerve head (thus crossing over the central and peripheral retinal regions) were used. The total number of RGCs in each retinal section was recorded and divided by the length of the entire retinal section. The quantifications were performed by individuals blinded to the experimental conditions.

### Optomotor Response-Based Visual Acuity Test

The optomotor reflex-based spatial frequency threshold test was performed as described previously to measure the visual acuity of mice (Chen et al., 1998). A mouse was placed on a pedestal in the center of an area surrounded by four LCD screens (Acer 15-inch) that displayed black and white stripes in a grating pattern, which rotated clockwise or counter-clockwise. Each eye was tested separately based on the direction of the rotating stripes. Two observers watched the mouse's tracking behavior, and a positive response was determined when the mouse head followed the grating movement. The rotation speed and contrast were kept constant. The width of the bars was varied to test how fine the mouse could see, and the corresponding bar width was calculated in term of cycle/degree where 0.4~0.5 cycle/degree reflects normal visual acuity in mouse. The responses were measured before and after treatment by individuals blinded to the group or treatment of the mouse. Mice with bleeding or inflammation after surgical procedures were excluded from the analysis. The exclusion criteria were established before the experiment.

### Light/Dark Preference Test

The light/dark preference test is based on the propensity of mice to prefer dark over bright environments (Rao et al., 2002). A mouse was placed in a box with a light and a dark chamber connected by a small opening to allow free transitions between these two chambers. Mice that can see light show a preference for the dark chamber while blind mice would have no preference. No eyes were covered during baseline measurement while the contralateral eyelid was sutured to prevent the uninjured eye from seeing when tested after crush. Each mouse was kept in the box for 5 minutes and the time it spent in the dark chamber was calculated as a percentage of the total time in the box. In a preliminary study, it was shown that mice with both eyes sutured exhibited no preference for the dark chamber as the blind mice, an indication of effective blockade of light perception by suturing the eye as it was reported (Lu et al., 2020).

### Electroretinography and Visual Evoked Potential

Electroretinography of positive scotopic threshold response (pSTR) and visual evoked potential (VEP) were measured as described previously (Chen et al., 1998; Juárez-Mercado et al., 2020). For pSTR recording, mice were dark adapted for 6-12 h before experiment started; while for VEP recording, mice were briefly adapted to dim red light (5-10 min) beforehand. Mice were anaesthetized with ketamine/xylazine (100 mg kg-1 and 20 mg kg-1). A drop of 1% tropicamide ophthalmic solution (Bausch & Lomb Inc., Tampa, FL, USA) was applied to dilate the pupils. Dim red light was on throughout the procedure and mice were kept on a built-in warming platform in the Ganzfield ColorDome (ColorDome LabCradle mouse ERG testing, Diagnosys LLC) to prevent hypothermia. Gold wire electrodes contacting both corneas were used to record pSTR with a reference and a ground needle electrode inserted subcutaneously between the eyes and at the base of the tail, respectively. Lights at intensities of 6.57E-5 cd·s/m² and 1.7E-4 cd·s/m² were flashed in the dome to elicit pSTR per intensity. An average of 40 responses were recorded by the ERG system (Espion Electroretinography System, Diagnosys LLC) and the b-wave amplitude was measured from the baseline to the positive peak. For VEP, electrode needles were placed subcutaneously at the snout, between the ears, and at the base of the tail. The contralateral eye was covered with a dark patch when recording the ipsilateral eye and vice versa. VEP was stimulated by 100 flashes at 3.0 cd·s/m² and the averaged response was recorded by the ERG system.

### Single-Nuclei RNA Sequencing

Single-nuclei RNA sequencing was performed based on a previously described method (Day et al., 2010). Retinas from the ONC eyes were collected from 6 control and 6 CKO^{Dnmt3a+/-} mice, respectively, at 2 days after injury (1 eye/mouse). To extract nuclei, frozen retinal tissues were homogenized in a Dounce homogenizer in 1 ml NP-40 lysis buffer and pelleted at 500 rcf for 5 min. The nuclei were stained for RBFOX3/NeuN (1:300, Sigma, #FCMAB317PE or #MAB377A5) and PTPRC/CD45 (1:300, BD Pharmingen, clone 30-F11) to enrich RGCs and immune cells, respectively. NeuN+ and CD45+ single nuclei were collected in separate tubes using a flow cytometer, and again pelleted at 500 rcf for 5 min. The nuclei were resuspended in 0.04% non-acetylated BSA/PBS solution, adjusted to a concentration of 1000 nuclei/μL, and loaded into a 10× Chromium Single Cell Chip (10× Genomics, Pleasanton, CA) with a targeted recovery of 8000 nuclei. As a result, sequencing was performed for a total of 17,895 nuclei in the control group (Neun+, 9210; CD45+, 8685) and 16,683 nuclei in the CKO^{Dnmt3a+/-} group (Neun+, 8235; CD45+, 8448). Single nuclei libraries were generated using the Chromium 3′ V3.1 platform (10× Genomics, Pleasanton, CA) following the manufacturer's protocol, and sequenced on an Illumina NovaSeq at the Bauer Core Facility at Harvard University.

### Single-Nuclei RNA-Seq Data Analysis

Data were processed in Cell ranger (Ji et al., 2022) to generate the output folder with barcodes, matrix and genes files. Those files were used to generate Seurat object using CreateSeuratObjecto function in Seurat package (Ferinelli et al., 2014; Chen et al., 2020). Data was filtered to remove doublets, low quality cells and empty droplets and performed filtering based on features/counts/mitochondrial genes/ribosomal genes following the standard Seurat pipeline (Jackson-Grusby et al., 2001). After data quality control and filtering, a total of 34,102 nuclei from both experimental groups were used for downstream analysis. Sequentially, data were normalized using the “NormalizeData” method, 2000 highly variable features were selected, data were centered, scaled, and clustered with Louvain algorithm (Kaneda et al., 2004). UMAP was chosen as a non-linear dimensionality reduction approach (Vong et al., 2011).

DimPlot( ) and FeaturePlot( ) functions were used in Seurat as visualization methods for the data annotation. By obtaining the differentially expressed genes list with the function FindAllMarkers(only.pos=TRUE, min.pct=0.25, log 2FC.threshold=0.25), manual annotation was performed for the dataset clusters (object$seurat_clusters) upon integrating the conditions (Control and CKO^{Dnmt3a+/-}) The integration was performed using the IntegrateData( ) function available in Seurat v4. Retinal ganglion cells (RGCs), immune cells, Müller glia, amacrine cells, horizontal cells, bipolar neurons, astrocytes, retinal pigment epithelium, rods, and cones were identified using the expression of genes known to be selective markers for each cell class (Tran et al., 2019). The immune cells population contains microglia/macrophages (Klra17+, C1qc+. Cx3cr1+. Tmem119+). T helpers (H2-Ab1+, Cd74+), T cells (Grap2+, Itgb7+). As the total number of immune cells in the populations was small, they were labeled as ‘Immune’ for the downstream analysis. FindAllMarkers( ) function was used to identify the gene expression dynamics between the conditions on total RGC subset of the dataset.

The RGCs were reclustered and normalized using the SCTransform method (Wassle et al., 2006) and also used the previously identified type-specific markers for RGC types identification (Barnstable et al., 1984). With the type markers list, the AverageExpressiono Seurat function was used to obtain the gene expression matrix and visualized it using the DotPlot(function. Upon identification based on the gene expression. 35 clusters were identified that contained 42 out of 45 known RGC types (Barnstable et al., 1984). In detail, from those 42 types, the resolution could be reached for 21 types in 24 separate clusters (1:1), with the other 21 types represented in 11 clusters, each of which contained 2-4 RGC types (W3D1|W3L1, M1a|M2, alpha OFF-T|alpha ON-T). The reason the resolution of RGC types could not be reached as published by Tran et al (Barnstable et al., 1984) is likely due to the relatively small number of cells available for this analysis.

To visualize RGC type-specific marker gene expression, a matrix of RGC Louvain clusters (object$seurat_clusters) was generated with the expression values for the query gene of interest. The gene expression ‘n’ from 0 to 1 was normalized for every gene k independently per cluster c (formula 1) and generated the values of gene patterns expression p for the gene patterns m (Gene1+Gene2+, Gene1+Gene2+Gene3-, etc.) by summarizing the normalized gene expression values ‘n’ per cluster c (formula 2). Upon receiving the values per pattern showing the gene pattern expression p, those were normalized from 0 to 1 per pattern m independently for every cluster c. The resulting matrix of normalized gene expression patterns ‘N’ was visualized using the ggplot2 geom_tile( ) (Perry et al., 1984).

$${n}_{k , c} = ( {x}_{k , c} - min k ) ( max k - min k ) \qquad Formula 1$$

$${p}_{m , c} = {n}_{{m}_{1} , c} \pm {n}_{{m}_{2} , c} \pm {n}_{{m}_{y} , c} \qquad Formula 2$$

$${N}_{m , c} = ( {p}_{m , c} - min m ) ( max m - min m ) \qquad Formula 3$$

To unravel cell-cell interactions, CellChat package (Cho et al., 2005) was used. Two parallel analyses were performed between the conditions: 1) merged RGC clusters vs. every other cluster in the dataset (immune cells, Müller glia, amacrine cells, horizontal cells, bipolar neurons, astrocytes, retinal pigment epithelium, rods, and cones): 2) subset RGC and performed the same type of the analysis for RGC clusters.

The analysis included the CellChat approach to generate total incoming/outgoing signaling and chord diagrams between Control and CKO^{Dnmt3a+/-} cells with additional settings. Those included type=“truncatedMean”, trim=0.1, raw.use=FALSE, population.size=TRUE for computeCommunProb( ) function. Upon generating the data, data of horizontal cells, bipolar neurons, astrocytes, retinal pigment epithelium, rods, and cones were merged and labeled as ‘rest. For the results, outgoing signaling was demonstrated using pattern=‘outgoing’ setting for the netAnalysis_signalingRole_heatmap( ) function and chord diagrams using netVisual_aggregate( ) function. The computational probability was exported to enable the communications comparisons between the cell types and conditions that were stored in the object as cellchat_object@netP$centr$pathway_name$outdeg and performed 0 to 1 normalization to visualize the data.

To perform GSEA pathway analysis, the escape package (Pemet et al., 2014) was used. The C5 pathways was loaded from the Molecular Signatures Database (Conceicao et al., 2019; Shi et al., 2018) (‘GOBP_NEURON_DEATH’, ‘GOBP_DENDRITE_EXTENSION’, ‘GOBP_OPTIC_NERVE_MORPHOGENESIS’, ‘GOBP_AXON_DEVELOPMENT’. ‘GOBP_REGULATION_OF_AXONOGENESIS’. ‘GOBP_RESPONSE_TO_AXON_INJURY’, ‘GOBP_INFLAMMATORY_RESPONSE’, ‘GOBP_INFLAMMASOME_COMPLEX_ASSEMBLY’, ‘GOBP_ACUTE_INFLAMMATORY_RESPONSE’. ‘GOBP_CYTOKINE_PRODUCTION_INVOLVED_IN_INFLAMMATORY_RESPONSE’, ‘GOBP_NEUROINFLAMMATORY_RESPONSE’) and performed ssGSEA enrichment (Conceicao et al., 2019). The ggplot2 and ggpubr packages (Ridder et al., 2006) were used for the data visualization.

Then, the pathways used for GSEA analysis were generalized into three programs: neuronal injury, inflammation, and regeneration. The neuronal injury program included ‘GOBP_NEURON_DEATH’ and ‘GOBP_RESPONSE_TO_AXON_INJURY’ pathways. The inflammation program included ‘GOBP_INFLAMMATORY_RESPONSE’, ‘GOBP_INFLAMMASOME_COMPLEX_ASSEMBLY’, ‘GOBP_ACUTE_INFLAMMATORY_RESPONSE’, ‘GOBP_CYTOKINE_PRODUCTION_INVOLVED_IN_INFLAMMATORY_RESPONSE’, and ‘GOBP_NEUROINFLAMMATORY_RESPONSE’ pathways. The regeneration program included ‘GOBP_DENDRITE_EXTENSION’, ‘GOBP_OPTIC_NERVE_MORPHOGENESIS’, ‘GOBP_AXON_DEVELOPMENT’, and ‘GOBP_REGULATION_OF_AXONOGENESIS’ pathways.

To normalize the contribution of the pathways to their corresponding programs from 0 to 1 (due to different average expressions and the amount of the pathways used), the matrix of normalized pathway average expression ‘A’ was generated for every pathway k per cluster c, using the pathway average expression ‘A’ data generated with GSEA analysis approach (formula 4).

$${A}_{k , c} = ( {a}_{k , c} - min k ) ( max k - min k ) \qquad Formula 4$$

Then, the pattern average expression ‘m’ was quantified for pathway mode y per cluster c as a summation of n pathways average expression A per cluster c with n=5 for injury, n=2 for inflammation, and n=4 for regeneration program (formula 5).

$${m}_{y , c} = {\sum }_{i = k}^{n} {A}_{i , c} = {A}_{k , c} + {A}_{{k}_{1} , c} + {A}_{n , c} \qquad Formula 5$$

Finally, the 0 to 1 normalized program average expression ‘M’ was quantified for pathway mode y per cluster c as a max/min normalization of program average expression m (formula 6).

$${M}_{y , c} = ( {m}_{y , c} - min y ) ( max y - min y ) \qquad Formula 6$$

The resulting matrix of RGC subtypes and programs was visualized using geom_point( ) and geom_line( ) functions of ggplot2 package with the programs separated using ~facet_wrap( ) function. The 3D visualization was performed for neuronal injury/inflammation/regeneration patterns using the plotly package (Extended Data HTML File), and 2D embeddings for injury/regeneration and inflammation/regeneration programs to demonstrate the distribution of RGC subtypes. For 2D and 3D embeddings, the values used are quantified as a difference of normalized program average expression M for pathway mode y per cluster c between CKO^{Dnmt3a+/-} and Control conditions.

### Whole Genome Bisulfite-Seq Analysis

DNA used for bisulfite sequencing were extracted from RGCs isolated from the injured eye at 2 days post-ONC. For each group, RGCs from 2 mice retinas were combined for DNA extraction (n=2 mice/group, 1 eye/mouse). Bisulfite sequencing and data analysis was performed by Novogene. Basic statistics on the quality of the raw reads was processed by FastQC (fastqc_v0.11.5) (Ridder et al., 2006). The read sequences produced by the Illumina pipeline in FASTQ format were pre-processed through Trimmomatic (Trimmomatic-0.36) software (Tang et al., 2020) using the parameter (SLIDINGWINDOW:4:15; LEADING:3, TRAILING:3: ILLUMINACLIP: adapter.fa: 2: 30: 10; MINLEN:36). The remaining reads that passed all the filtering steps were counted as clean reads and all subsequent analyses were based on this. Finally, FastQC was used to perform basic statistics on the quality of the clean data reads. Bismark software (version 0.16.3) (Jacobi et al., 2022) was used to perform alignments of bisulfite-treated reads to a reference genome (−X 700 --dovetail). The reference genome was first transformed into bisulfite-converted version (C-to-T and G-to-A converted) and then indexed using bowtie2 (Hahn et al., 2023). Sequence reads were also transformed into fully bisulfite-converted versions (C-to-T and G-to-A converted) before they were aligned to similarly converted versions of the genome in a directional manner. Sequence reads that produced a unique best alignment from the two alignment processes (original top and bottom strand) were then compared to the normal genomic sequence and the methylation state of all cytosine positions in the read was inferred. The same reads that aligned to the same regions of genome were regarded as duplicated ones. The sequencing depth and coverage were summarized using deduplicated reads. The results of methylation extractor (bismark_methylation_extractor. -- no_overlap) were transformed into bigWig format for visualization using IGV browser (Lim et al., 2016). The sodium bisulfite non-conversion rate was calculated as the percentage of cytosine sequenced at cytosine reference positions in the lambda genome.

To identify the methylation site, the sum Mc of methylated counts was modeled as a binomial (Bin) random variable with methylation rate. To calculate the methylation level of the sequence, the sequence was divided into multiple bins, with bin size is 10kb. The sum of methylated and unmethylated read counts in each window were calculated. Calculated ML was further corrected with the bisulfite non-conversion rate according to previous studies (Duan et al., 2015).

Differentially methylated regions (DMRs) were identified using the DSS software (Rheaume et al., 2023; Sun et al., 2011; Jin et al., 2021). The core of DSS is a new dispersion shrinkage method for estimating the dispersion parameter from Gamma-Poisson or Beta-Binomial distributions (Park et al., 2011). DSS possesses three characteristics to detect DMRs. First, spatial correlation. Proper use of the information from neighboring Cytosine sites can help improve estimation of methylation levels at each Cytosine site, and hence improve DMR detection. Second, the read depth of the Cytosine sites provides information on precision that can be exploited to improve statistical tests for DMR detection. Finally, without biological replicate, DSS combines data from nearby Cytosine sites and uses them as ‘pseudo-replicates’ to estimate biological variance at specific locations. According to the distribution of DMRs through the genome, the genes related to DMRs were defined as genes whose gene body region (from TSS to TES) or promoter region (upstream 2kb from the TSS) have an overlap with the DMRs. The rich factor was quantified as the number of DMR-related genes (input genes) divided by the number of pathway genes for each pathway, and the resulting parameters (rich factor, methylation type, and p-value) for the pathways selected from top-20 sorted by p-value (input genes≥20 per pathway) were visualized using geom_bar( ) and geom_point( ) ggplot2 functions.

### Bulk RNA-Seq Data Analysis

RNA used for bulk RNA sequencing were extracted from RGCs isolated from the injured eye at 2 days post-ONC (n=3 eyes/group, 1 eye/mouse). Raw data (raw reads) of .fastq format were first processed through in-house perl scripts by Novogene. In this step, clean data (clean reads) were obtained by removing reads containing adapter, ploy-N and low-quality reads from raw data. At the same time, Q20, Q30 and GC content of the clean data were calculated. All the downstream analyses were based on clean data with high quality.

Reference genome and gene model annotation files were downloaded from the genome website directly. The index of the reference genome was built using Hisat2 v2.0.5 and paired-end clean reads were aligned to the reference genome using Hisat2 v2.0.5 (Rao et al., 1993). The mapped reads of each sample were assembled by StringTie (v1.3.3b) (Carulli et al., 2021) in a reference-based approach. The reads numbers mapped to each gene was counted by featureCounts v1.5.0-p3 (Zhou et al., 2019). FPKM of each gene was calculated based on the length of the gene and reads count mapped to this gene. Differential expression analysis of 3 conditions/groups (3 biological replicates per condition) was performed using the DESeq2 R package (1.20.0) (Luck et al., 2021). DESeq2 provides statistical routines for determining differential expression in digital gene expression data using a model based on the negative binomial distribution. The resulting P-values were adjusted using Benjamini and Hochberg's approach for controlling the false discovery rate. Genes with an adjusted P-value≤0.05 found by DESeq2 were assigned as differentially expressed (DEGs).

The KEGG and GO pathway annotations of the DEGs were performed with ShinyGo (Li et al., 2022) version 0.76 with the FDR cutoff=0.05. The top 10 up or down-regulated pathways that are relevant to RGCs were visualized in dotplots (input genes≥5 per pathway, sorted by fold enrichment). For GSEA pathway analysis of the DESeq2 output, the clusterProfiler (Garcia et al., 2018), msigdbr (Musada et al., 2022), and org.Mm.eg.db (Johnson et al., 2014) were used. For the pathways, Hallmark category (category=“H”) (Ikeda et al., 2004) was used, and upon filtering and sorting data based on log 2FC values, the table was generated with the GSEA( ) function. The top RGC-relevant pathways were visualized using the ggplot2 package (setSize≥5, sorted by Normalized enrichment score, NES).

The volcano plot was generated with the DESeq2 output using the Enhanced Volcano package (Kajita et al., 2007). The genes labeled on the plot were the result of two approaches: 1) the most significant genes appearing in DESeq2 output by log 2FC and adjusted p-value: 2) genes appearing significant in the single nuclei RNA-seq data, pathways related to differentially methylated regions, and experimental data.

### Cell-Cell Interactions Atlas Generation

Upon generating the CellChat objects, the indegree and outdegree representing the incoming and outgoing signaling, respectively were exported. Those were used to generate the matrix consisting of cell barcodes as row names, while pathways (incoming), pathways (outgoing), ligands—ligand-receptor pairs:pathways (outgoing), receptors—ligand-receptor pairs:pathways (incoming) were used as column names. The CellChat object exported values were used to fill in the matrix. The following objects generated were formatted to follow the unified structure of the objects to be deposited to the Broad Institute's Single Cell Portal.

### Data Availability

The raw and processed data of bisulfite, bulk, and single nuclei transcriptomic experiments generated in this study have been deposited in the GEO database under accession codes: GSE229033 (bulk), GSE229034 (bisulfite), GSE228627 (single-nuclei). The single nuclei data can be explored at the Broad Institute's Single Cell Portal: singlecell.broadinstitute.org/single_cell/study/SCP2321/. The cell-cell interactions can be explored at: singlecell.broadinstitute.org/single_cell/study/SCP2333/for the Control, and singlecell.broadinstitute.org/single_cell/study/SCP2351/for the CKO^{Dnmt3a+/-}.

### Code Availability

The code for reproducing the bioinformatical analysis is available on the following GitHub repository: github.com/mcrewcow/Lydia_ChenLab_RGC_pONC_retina.

### Intravitreal Injection in Mice

Procedure for intravitreal injection was essentially described (Chen et al., 1995). During intravitreal injection, a pulled microcapillary tube was inserted just behind the ora serrata in the mouse peripheral retina. The vitreous was then partially removed (about 2 μl) to enable the injection of 2 μl of solution into the vitreous chamber. For experiments using AAVs, mice were kept in housing for at least two weeks after the injection of AAV to ensure that the target genes were stably expressed. For tracing regenerating axons anterogradely, 2 μl of 2 μg/μl CTB (List Biological Laboratories, 104) was injected intravitreally 2-3 days before mice were sacrificed at various time points to assess the extent of nerve regeneration after ONC.

### Adeno-Associated Viruses (AAV) Constructs and Production

AAV serotype 2/2 (AAV2) was used in all virus-mediated knockdown experiments. For Dnmt3a gene knockdown, vectors were generated in-house where an AAV.Scramble (scrambled control (5′-cctaaggttaagtcgccctcgctcgagcgagggcgacttaaccttagg-3′; SEQ ID NO: 1) and two AAV.shRNAs targeting Dnmt3a (#1: 5′-cgctccgctgaaggaatatttctcgagaaatattccttcagcggagcg-3′ SEQ ID NO: 2 and #2: 5′-ggcatccactgtgaatgataactcgagttatcattcacagtggatgcc-3′ SEQ ID NO: 3) were designed using the GPP Web Portal (Broad Institute). The shRNA sequences were synthesized as single-stranded oligonucleotides, annealed, and inserted into pAAV-U6-sgRNA-CMV-GFP (Addgene #85451) at SapI and SapI sites. AAV2 were produced as described using a triple transfection approached in HEK293T cells in 15 cm dishes with polyethylenimine and were harvested after 60 h incubation, purified by iodixanol gradient ultracentrifugation, and quantified by quantitative PCR (titers: >1×10¹² genome copies per ml). AAV2/2.CASI.CRE.RBG (AAV.Cre) and AAV2/2.CASI.EGFP.RBG (AAV.GFP) (titers: >1.02×10¹² GC/mL) were purchased from the Gene Transfer Vector Core at Schepens Eye Research Institute.

### Example 1: RGC Axon Growth Enabled by Dnmt3a Deficiency

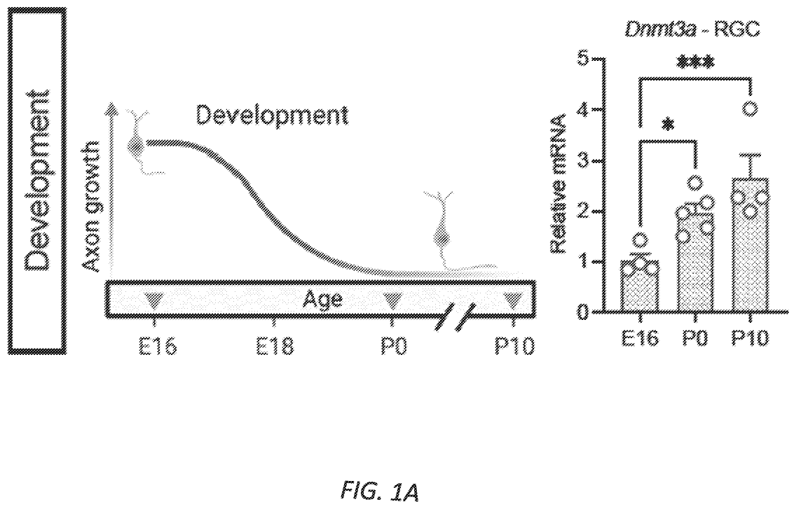

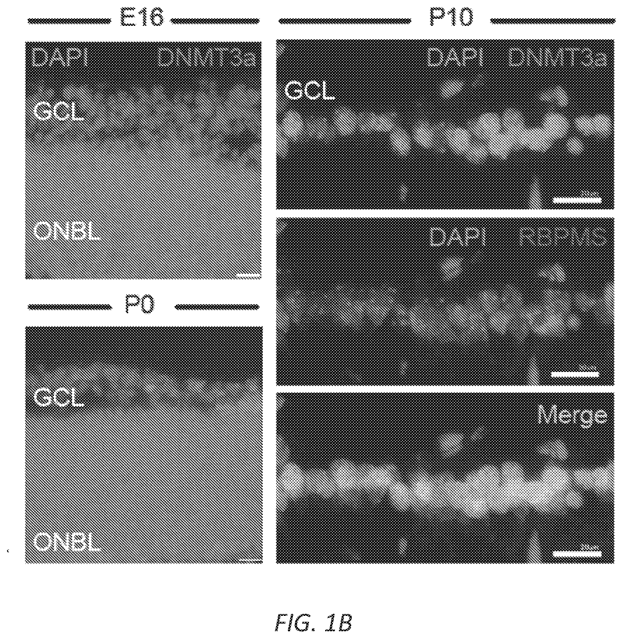

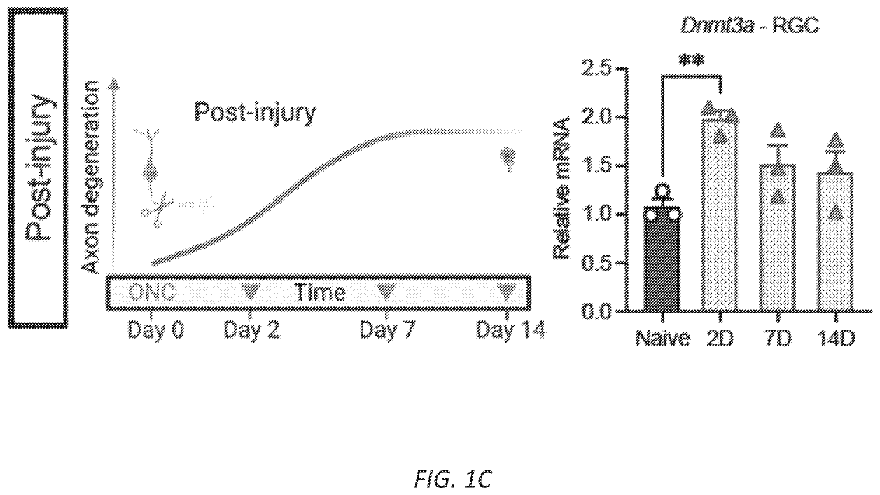

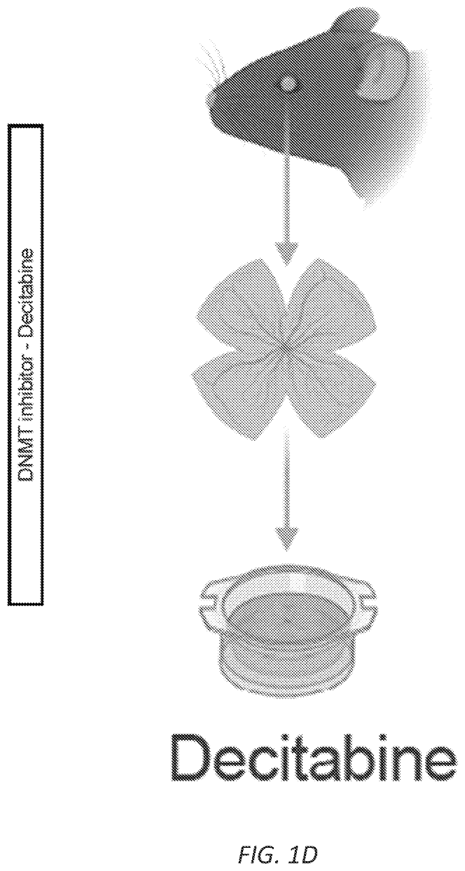

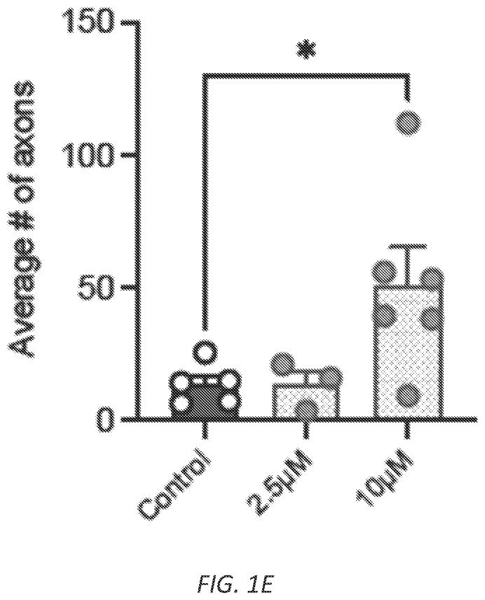

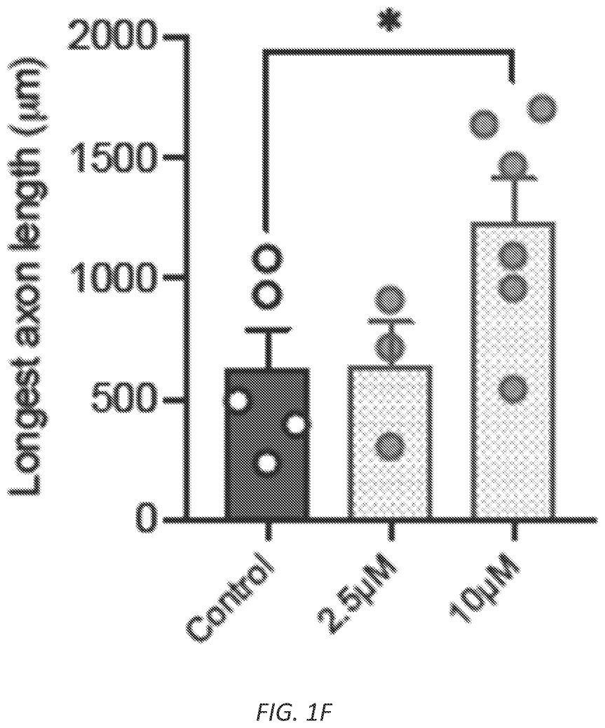

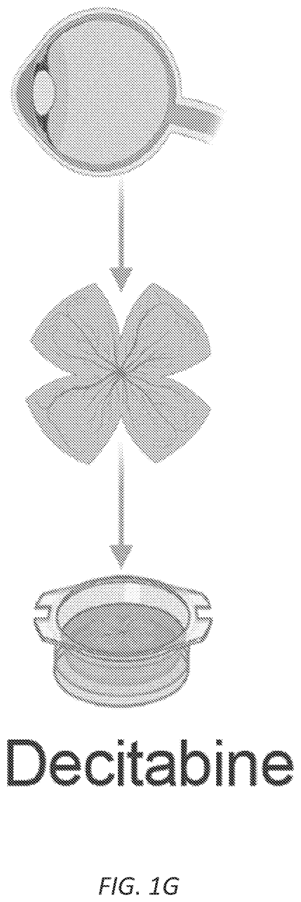

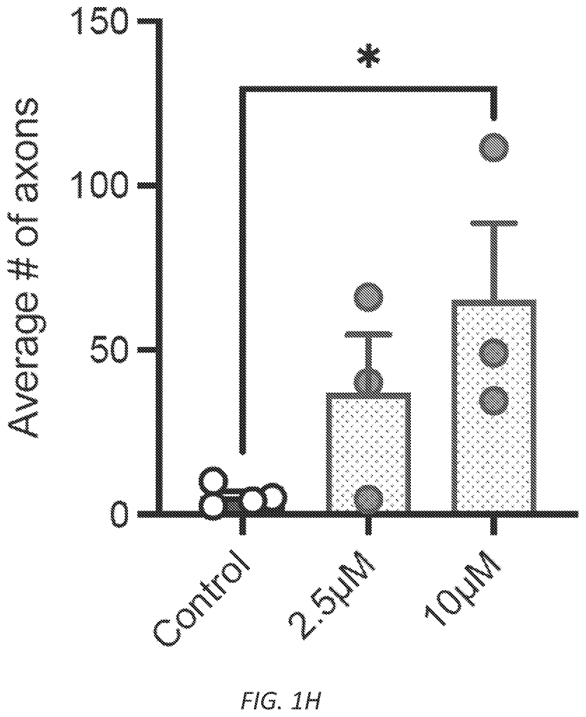

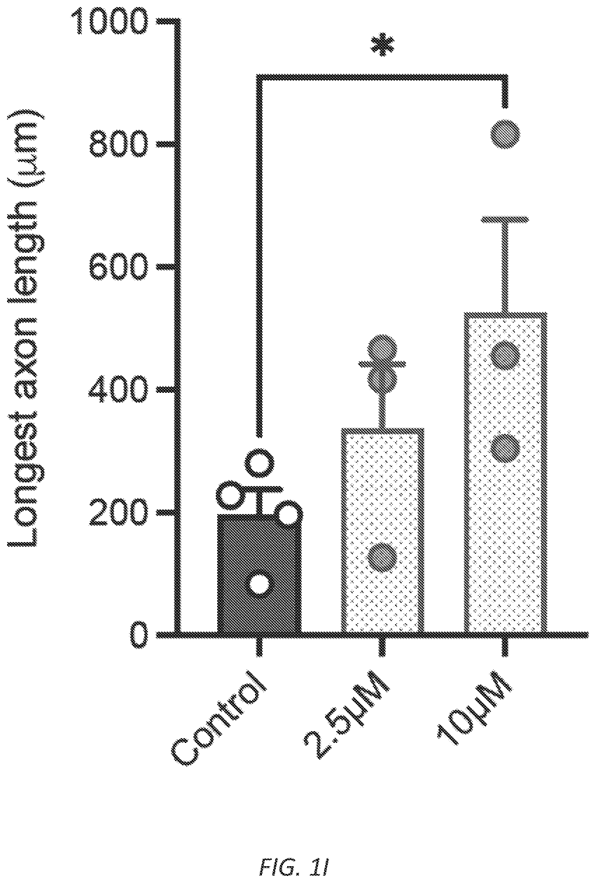

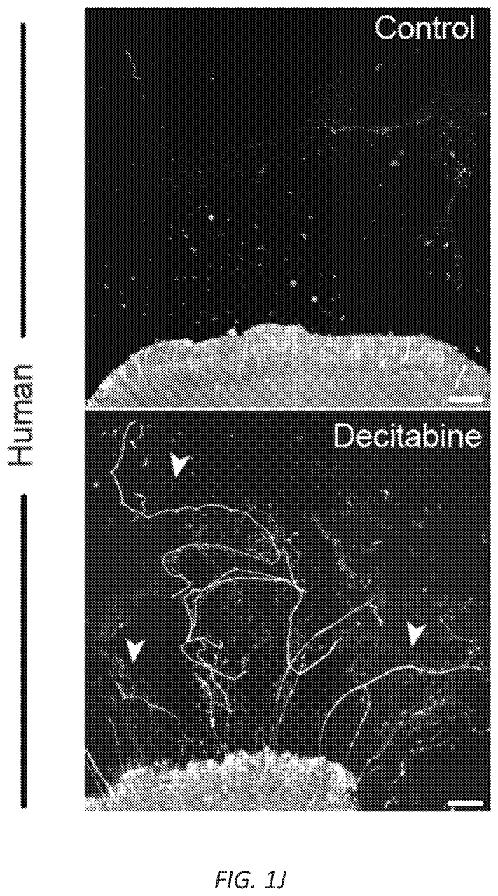

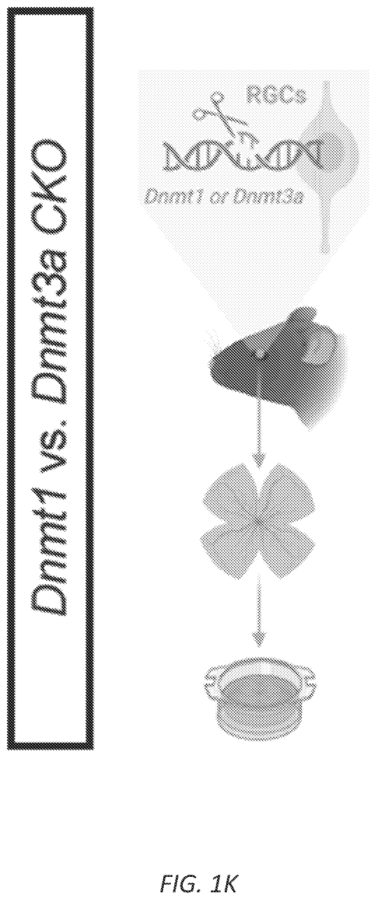

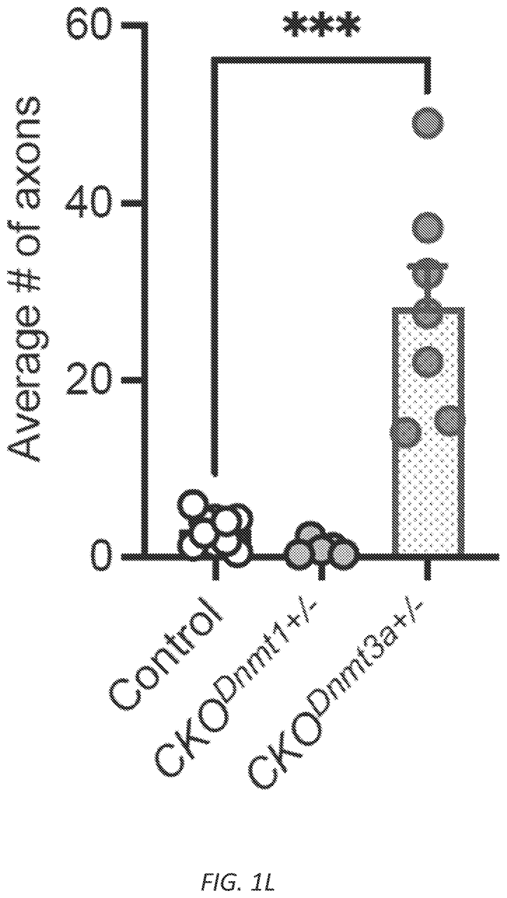

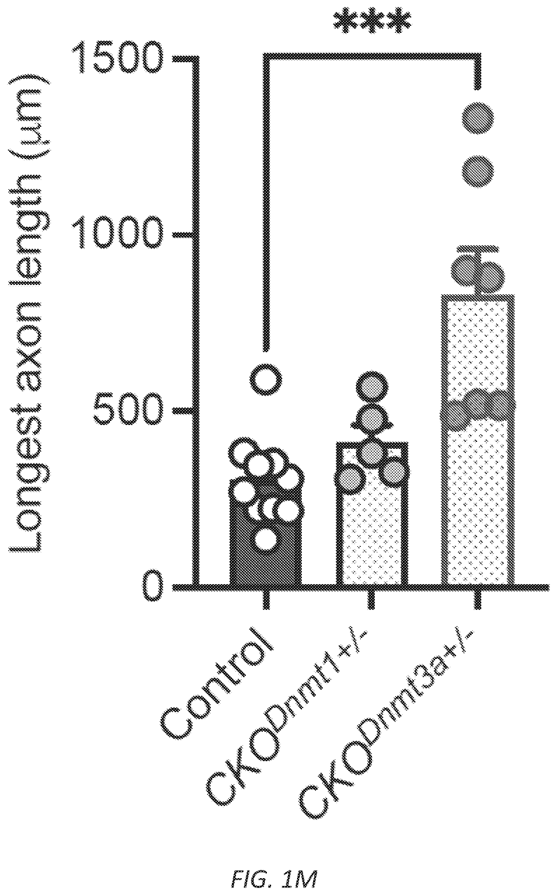

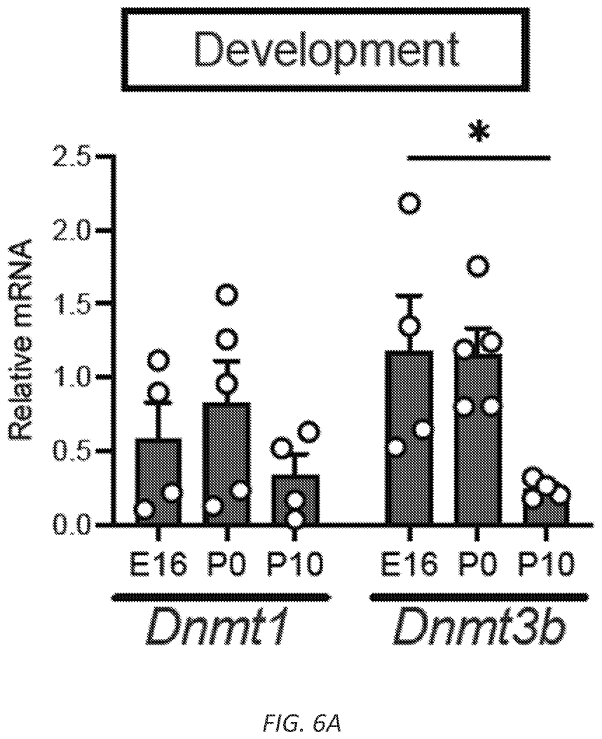

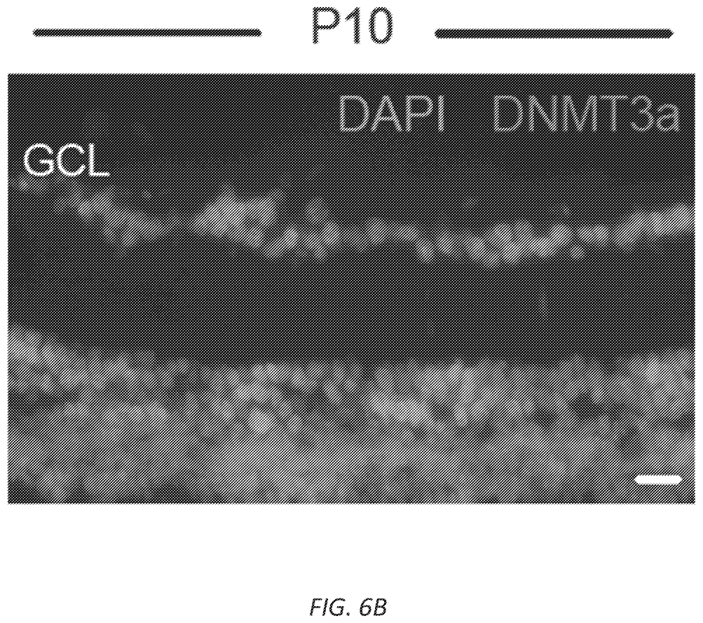

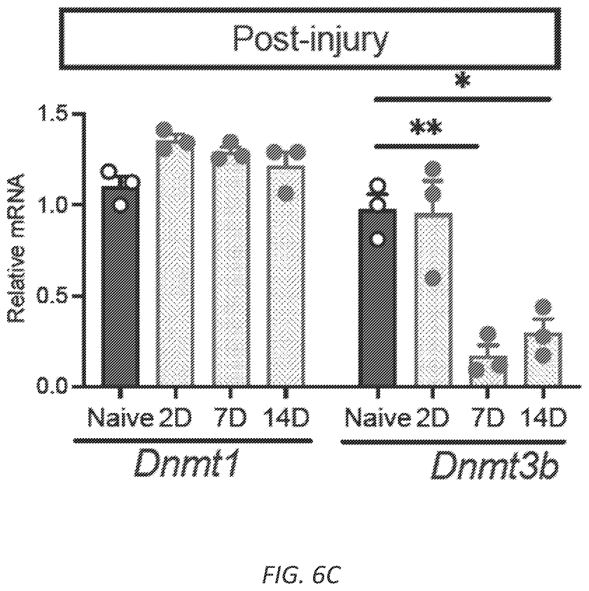

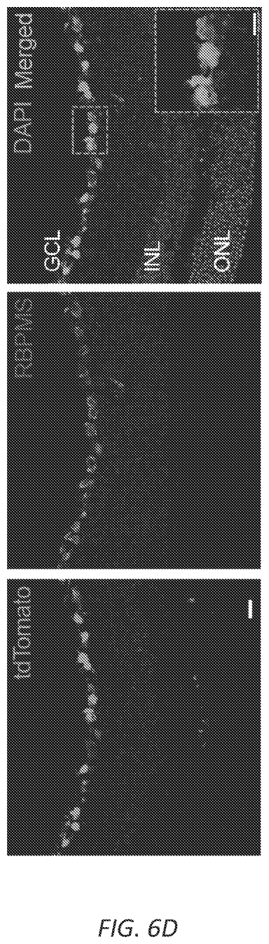

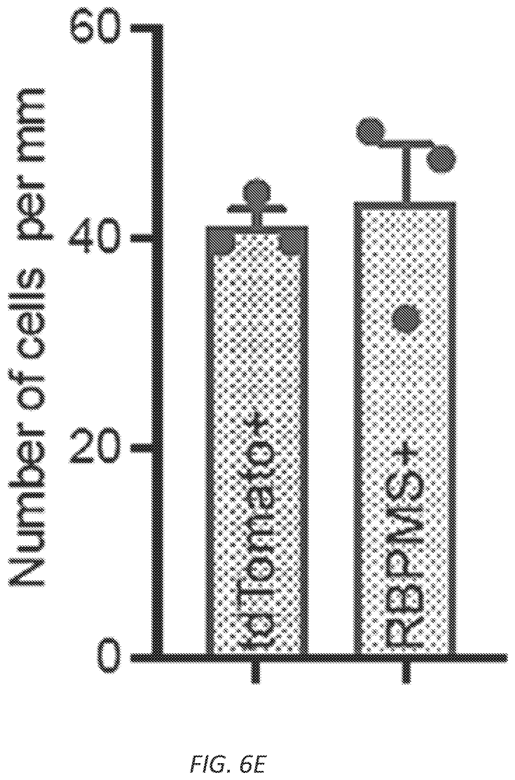

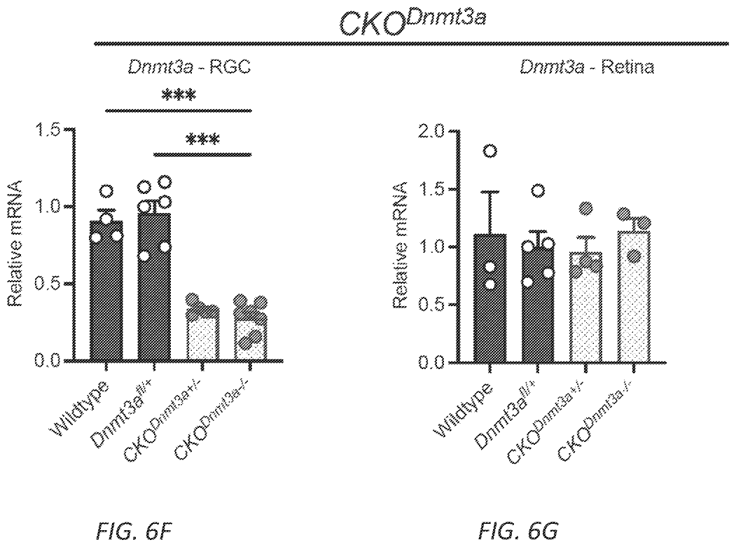

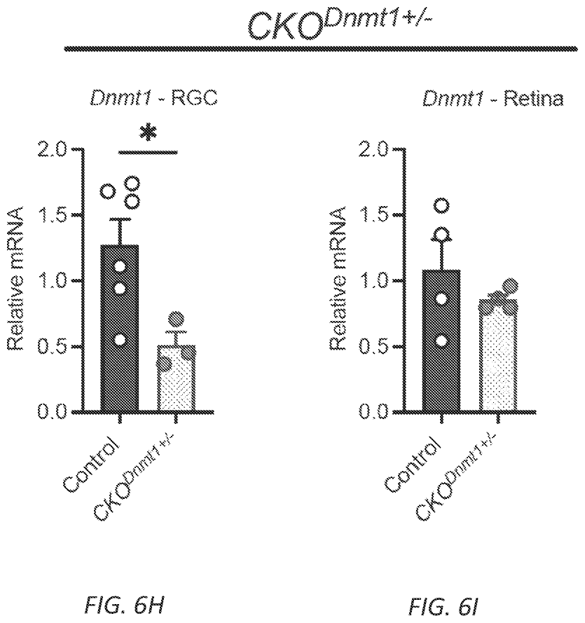

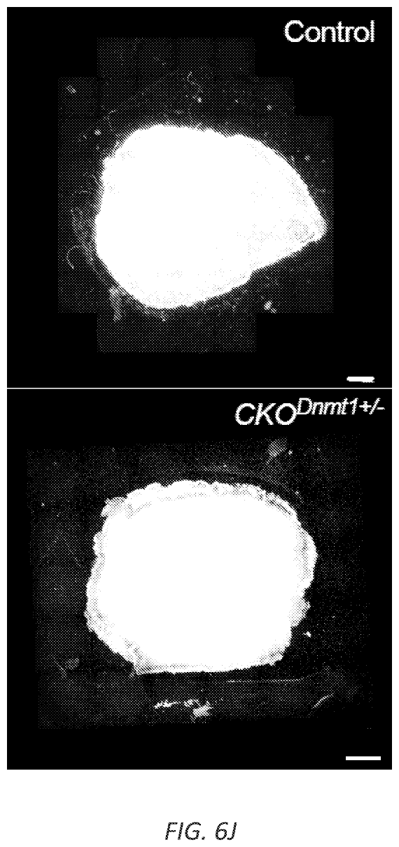

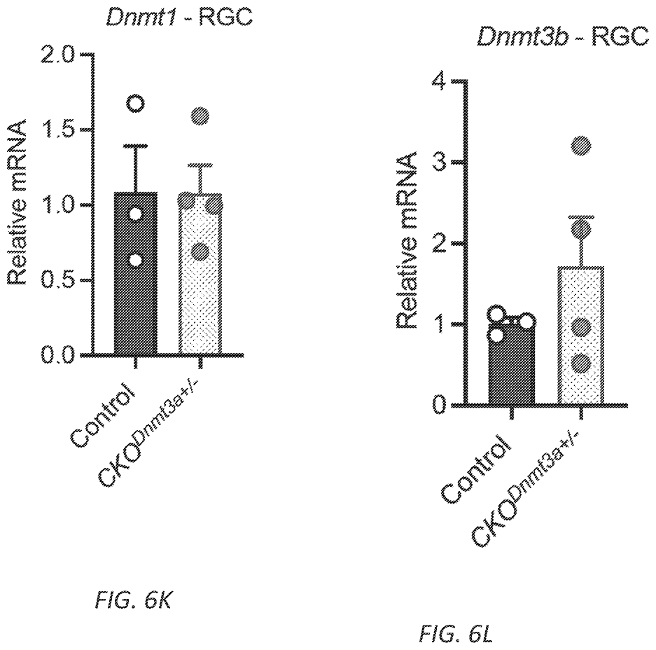

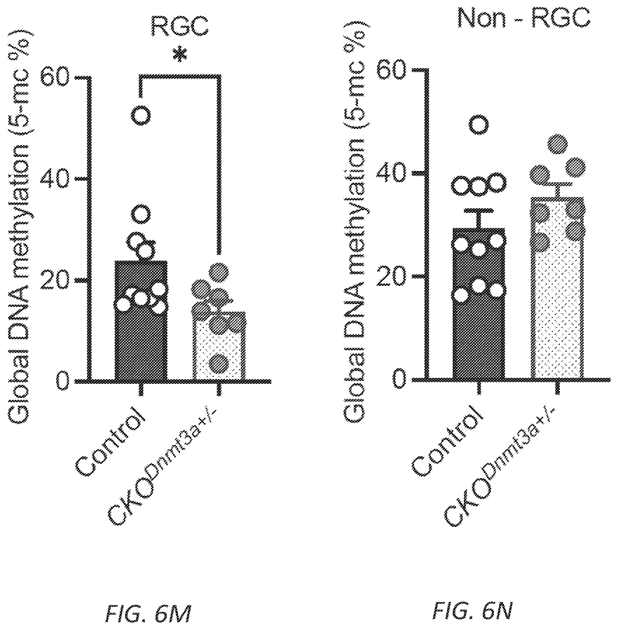

Mouse RGCs lose their intrinsic ability to regenerate axons around embryonic day 18 (E18), one day before birth (postnatal day 0; P0)⁵. To assess epigenetic mechanisms responsible for this decline, the expression patterns of DNA methyltransferases (DNMTs) were examined in developing RGCs of mouse pups aged E16 (before the time that RGCs lose their ability to regenerate axons), P0 (at the time when RGCs lose their ability to regenerate axons) and P10 (after the time that RGCs lose their ability to regenerate axons). Quantitative polymerase chain reaction (qPCR) of purified RGCs showed that the expression of Dnmt3a increased significantly from E16 to P10, which corresponded to the decline of RGCs' axon regenerative capacity (FIG. 1A). Other DNMTs, including Dnmt1 and Dnmt3b, did not exhibit this correlation (FIG. 6A). Immunohistochemistry confirmed that the expression of DNMT3a was absent in E16 RGCs or retina but intense in P10 retina (FIG. 1B and FIG. 6B), which was especially pronounced in the RGCs as shown by colocalization with RBPMS, an RGC marker (FIG. 1B). Moreover, Dnmt3a levels were increased in the RGCs two days after optic nerve crush injury (ONC; FIG. 1C), while Dnmt1 and Dnmt3b remained unchanged or downregulated (FIG. 6C). These data suggest a correlation between DNMT3a upregulation and the decrease of optic nerve regenerative capacity in mice.

To ascertain whether DNMTs affect RGC axonal regrowth, the effects of decitabine, a commercially available pan DNMT inhibitor (Juarez-Mercado et al., 2020) was assessed in vitro. Consistent with the lack of nerve regeneration, retinal explants of adult (>2 months old) wildtype mice and post-mortem human eyes showed minimal axonal outgrowth as revealed by β-III tubulin immunolabeling. In contrast, both mouse and human retinal explants treated with decitabine exhibited significant increase in the number and length of axonal growth (FIG. 1D-J).

To determine which DNMT(s) regulate the axon growth capacity in postnatal RGCs, a genetic approach was adopted. Two DNMT families play different roles in methylation: DNMT1 maintains the methylation patterns, whereas the DNMT3 family (especially DNMT3a and DNMT3b) establishes initial methylation patterns (Chen et al., 2020). Conditional knockout mice were generated that carried RGC-specific allele with abolished catalytic activity of Dnmt1 (CKO^{Dnmt1}) (Jackson-Grusby et al., 2001) orDnmt3a (CKO^{Dnmt3a}) (Kaneda et al., 2004). Vglut2-Cre transgenic mice can drive the expression of Cre recombinase in virtually all RGCs and sporadically cone photoreceptors, but not other retinal cell types (Vong et al., 2011: Tran et al., 2019). This was verified by crossing Vglut2-Cre transgenic mice with the Ai9 mouse line, which expressed the fluorescent reporter tdTomato from the Rosa26 locus in a Cre-dependent manner (R26-tdTomato). Approximate 90% of RBPMS+ RGCs and a small number of cones (Wassle et al., 2006), but not other retinal cells, were positive for tdTomato (FIG. 6D, E).

By generating CKO^{Dnmt1} and CKO^{Dnmt3a}, it was noted that the adult homozygous CKO^{Dnmt3a+/-} mice developed obesity and malocclusion, presumably because vGlut2 drives Cre expression in many CNS neurons. The mice also suffered a high mortality rate from anesthetics during surgical procedures. In contrast, heterozygous CKO^{Dnmt3a+/-} mice grew and bred normally without apparent health concerns or retinal structural changes. Therefore, the heterozygous CKO mice were chosen to as a focus. The RGCs were isolated using magnetic bead-conjugated Thy-1 antibody and detected significant downregulation of Dnmt3a mRNA in heterozygous CKO^{Dnmt3a+/-} RGCs compared to those from the littermate controls (FIG. 6F). In homozygous CKO^{Dnmt3a+/-} RGCs, a low level of Dnmt3a mRNA was detected, likely because Thy-1 is not exclusively expressed by RGCs in the rodent retinas (Barnstable et al., 1984; Perry et al_, 1984). The mRNA levels of Dnmt1 and Dnmt3a were downregulated selectively in the RGCs, but not in the entire retinas of CKO^{Dnmt1+/-} and CKO^{Dnmt3a+/-} mice, respectively (note that RGCs comprise ~1% of all retinal cells; FIG. 6F-I). The axonal growth capacity was examined using the retinal explant cultures derived from adult CKO^{Dnmt1+/-} and CKO^{Dnmt3a+/-} mice, and littermate controls that carried Cre without floxed alleles or floxed alleles without Cre (FIG. 1K-N). While the retinal explants of control mice showed minimal axonal growth, those from adult CKO^{Dnmt3a} mice exhibited an over 20-fold increase in axon number (FIG. 1L) and at least a 2-fold increase in average axon length (FIG. 1M) compared to control mice. The retinal explants of CKO^{Dnmt3a+/-} and CKO^{Dnmt3a+/-} mice revealed similarly increased number and rate of axon regeneration (data not shown), suggesting that the absence of one Dnmt3a allele was sufficient to unleash the barrier to axon regeneration. In contrast, retinal explants of CKO^{Dnmt1+/-} mice showed no significant improvement in axonal growth compared to controls (FIG. 1L AND M, FIG. 6J). It was also shown that the DNMT3a deficiency in CKO^{Dnmt3a+/-} RGCs did not have significant impact on the expression of Dnmt1 (FIG. 6K) or Dnmt3b (FIG. 6L). In agreement with the downregulation of Dnmt3a, diminished DNA methylation was detected in CKO^{Dnmt3a+/-} RGCs, but not other retinal cell types compared to controls (FIG. 6M, N). Therefore, DNMT3a, rather than DNMT1, negatively regulates the intrinsic program for RGC axon growth during maturation.

### Example 2: Optic Nerve Reinnervation and Restoration of Vision by Dnmt3a Deficiency

In vivo experiments were done to test if the RGC-specific DNMT3a deficiency restores axons' regenerative capacity in adult RGCs using the optic nerve crush (ONC) injury model. Adult CKO^{Dnmt3a+/-} mice, as well as their littermate Cre or floxed control mice, were subjected to ONC. Axonal regrowth was assessed by labeling RGC axons with cholera toxin B subunit (CTB) two days before mice were sacrificed (Lu et al., 2020; Cho et al., 2005). In control mice, CTB-positive axons were observed only proximal to the crush site (FIG. 2A) at all time points examined after ONC, confirming the failure of nerve regeneration. In contrast, numerous RGC axons regenerated across the lesion site for long distances in CKO^{Dnmt3a+/-} mice, with many growing more than 3 mm distal to the lesion by 14 days post ONC (FIG. 2A). Notably, the number of regenerated axons was so great that in most cases, it was impossible to count the number of regenerating axons in optic nerve sections. Thus, the axon regeneration was quantified by measuring the CTB-labeling immunofluorescent intensity at various distances posterior to the crush site. At all distances, at least a 3-fold increase in the intensity of CTB-labeling was observed in the CKO^{Dnmt3a+/-} mice compared to controls; the difference was >8-fold up to 750 μm distal to the lesion (FIG. 2B). Moreover, RGCs of CKO^{Dnmt3a+/-} mice displayed a significant increase in survival as shown by RBPMS-immunolabeling (50% vs 12% at 14 days post-ONC. FIG. 2C, D). No signs of RGC proliferation were detected as assessed by EdU incorporation assays (data not shown), suggesting that the increased RGC number was not due to the birth of new neurons or RGCs.

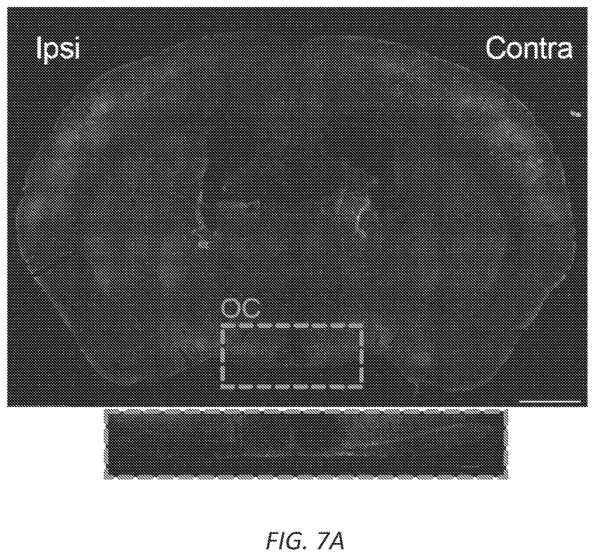

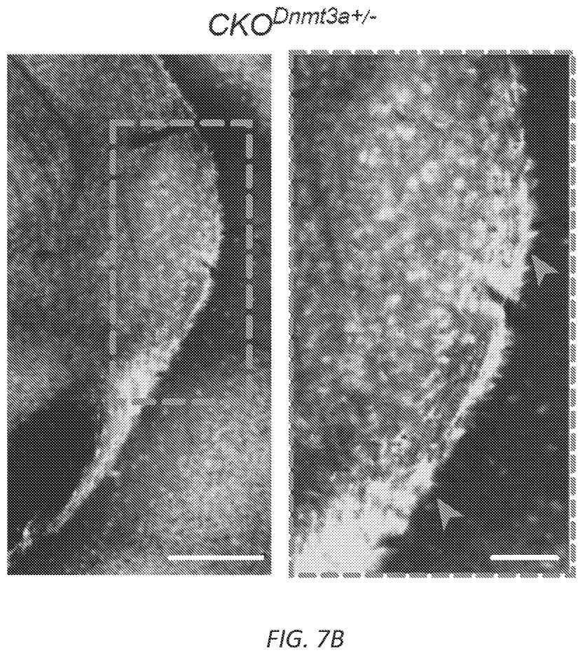

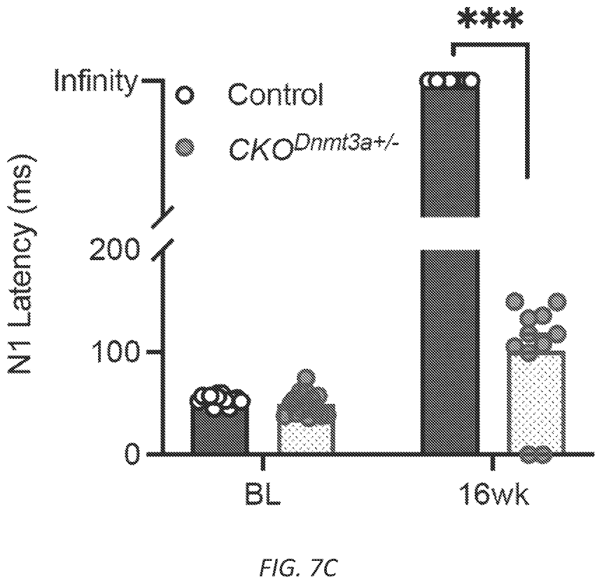

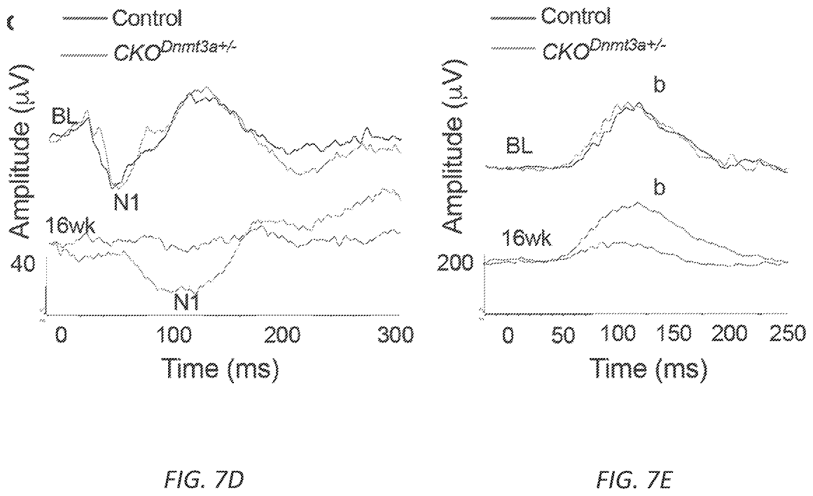

The regenerating axons continued to grow along the optic nerve over time. As early as 4 weeks following the injury, many regenerating axons grew past the optic chiasm, where a major barrier to RGC axon regeneration and target reinnervation was previously reported (Pernet et al., 2014: Conceicao et al., 2019). Greater fluorescent intensity of labeled axons entered the brain when examined at 8- and 16-weeks post-injury, suggesting continual growth of axons. By 16 weeks post-ONC, a large number of regenerating axons crossed the optic chiasm of the contralateral side (FIG. 2E, F). In contrast, no axons were evident in the optic chiasm (FIG. 7A), optic tract, and brain targets (data not shown) of control mice. Unprecedented numbers of regenerating axons were seen in the optic tract of CKO^{Dnmt3a+/-} mice, primarily extending along the side contralateral to the injury and reinnervating the dorsal and ventral lateral geniculate nuclei (LGN) as well as the pretectum (FIG. 2G and FIG. 7B). These findings showed that Dnmt3a deficiency in RGCs empowers long-distance and robust optic nerve regeneration and reinnervation into the central visual targets of the optic nerve in adult mice.

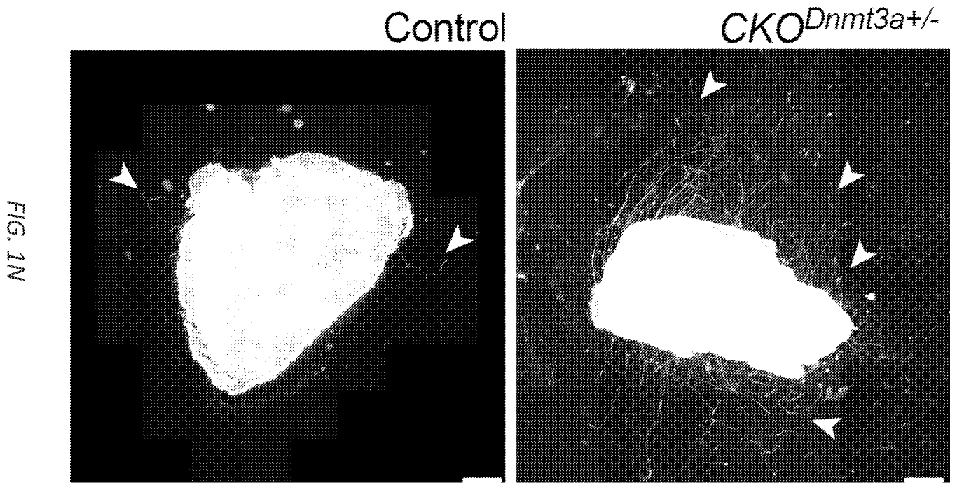

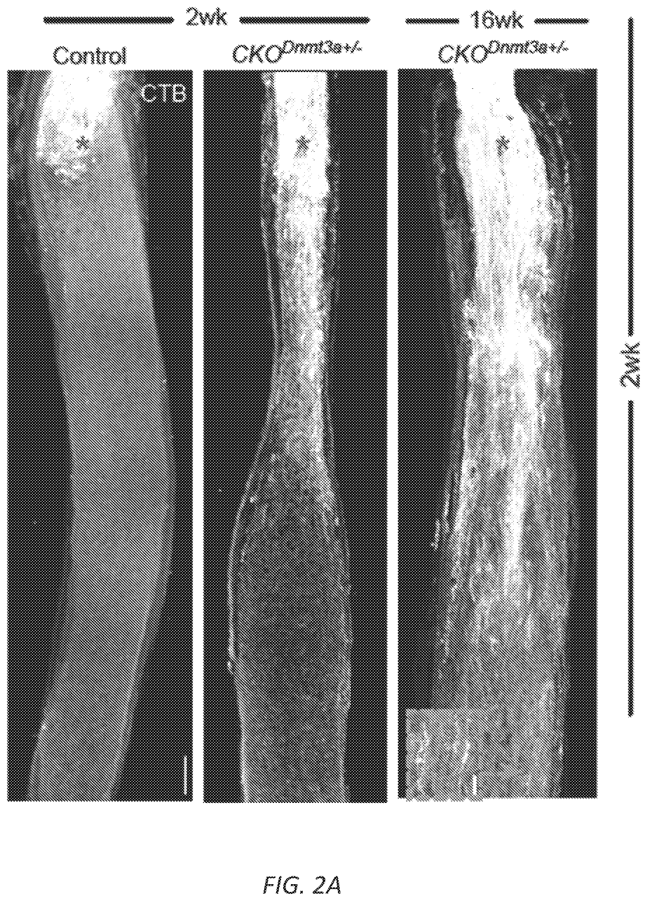

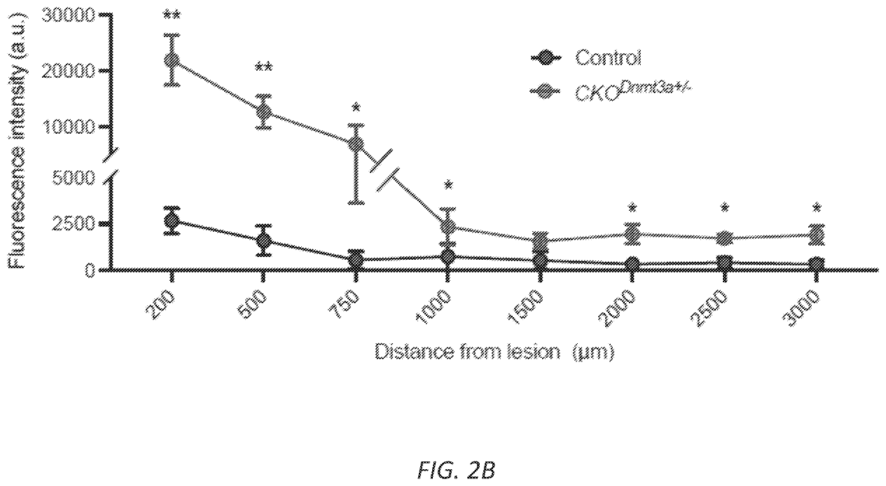

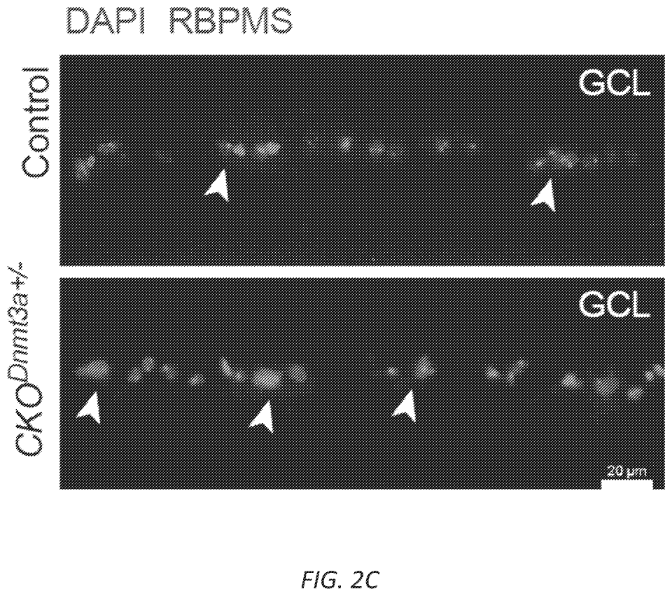

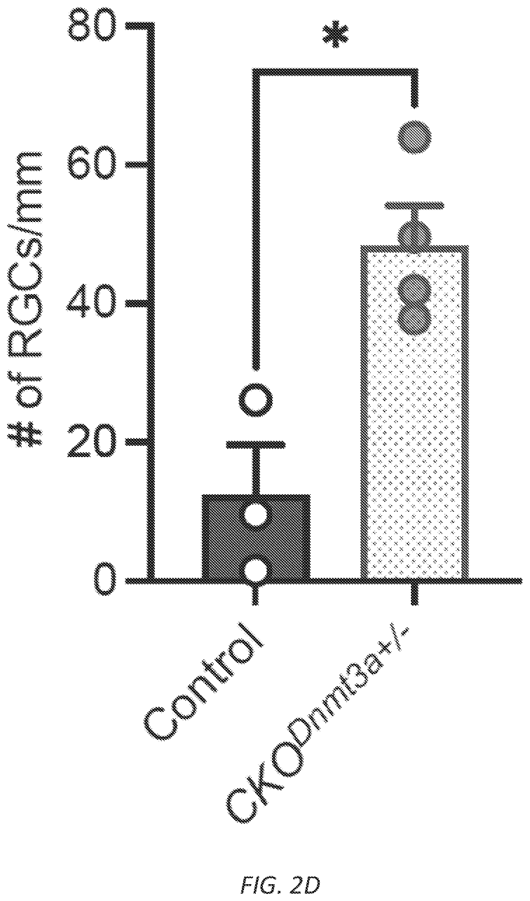

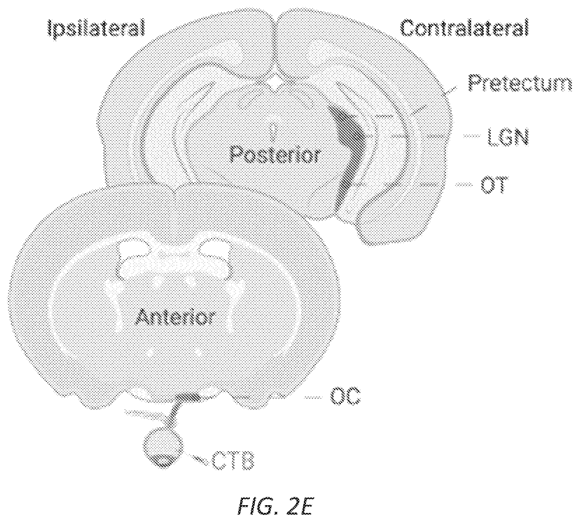

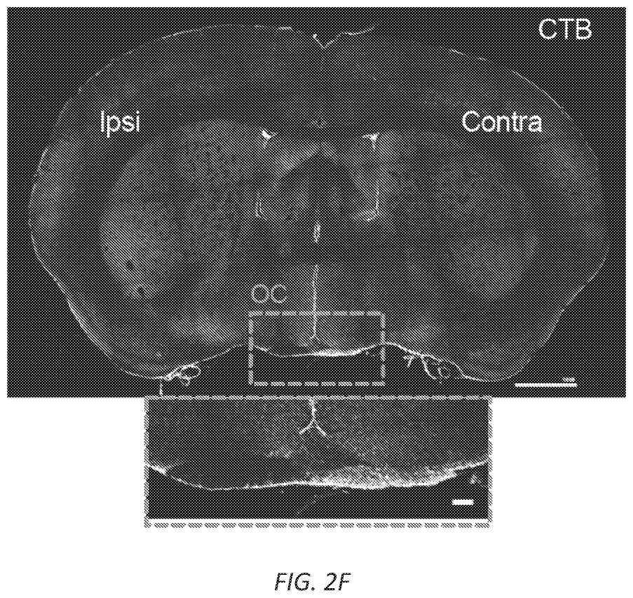

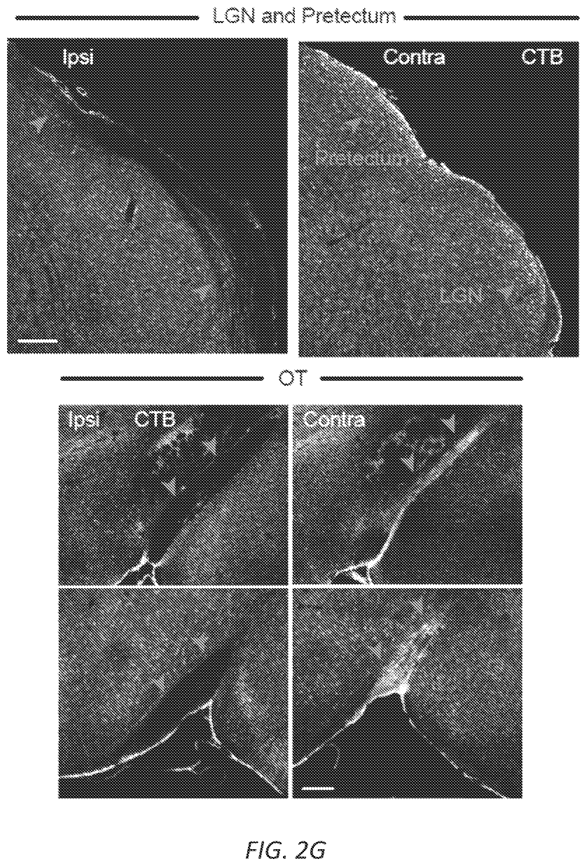

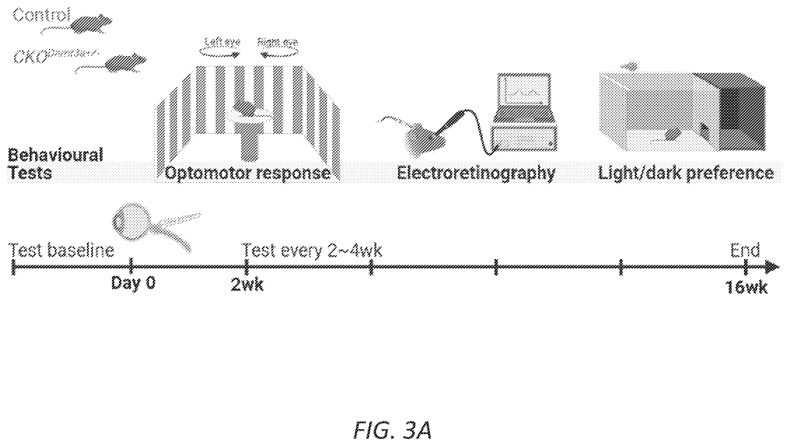

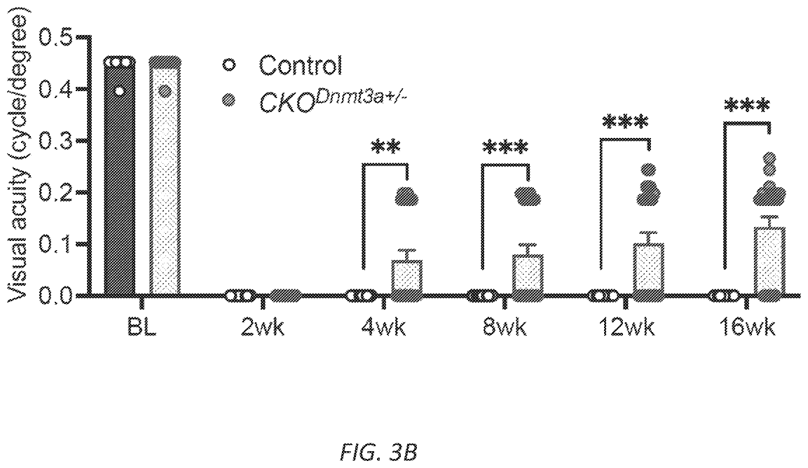

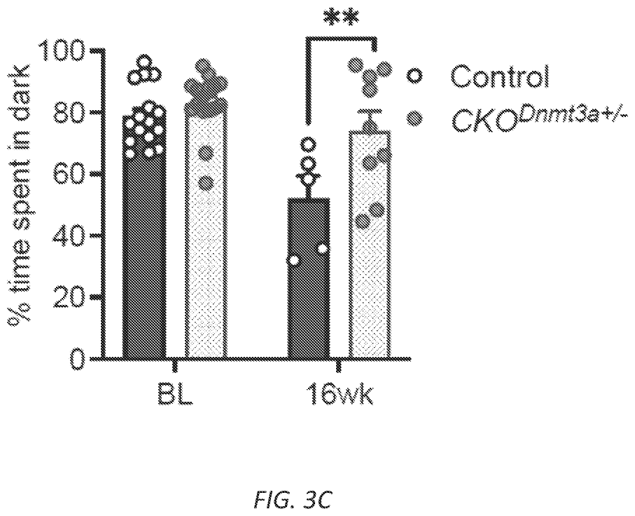

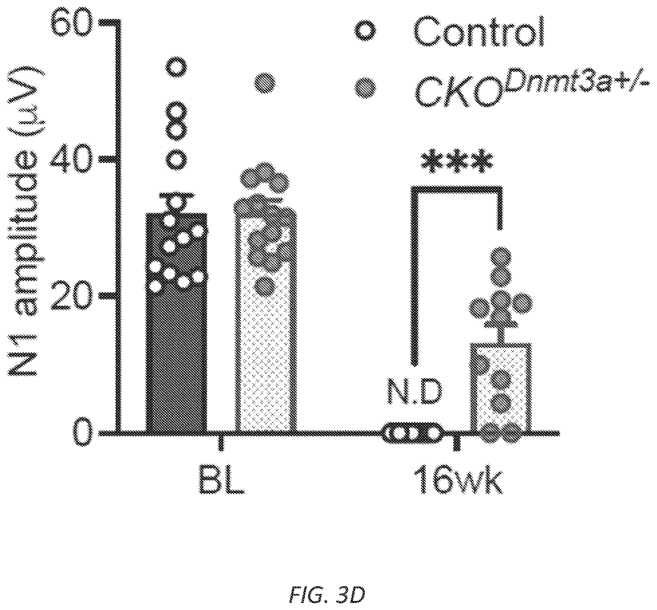

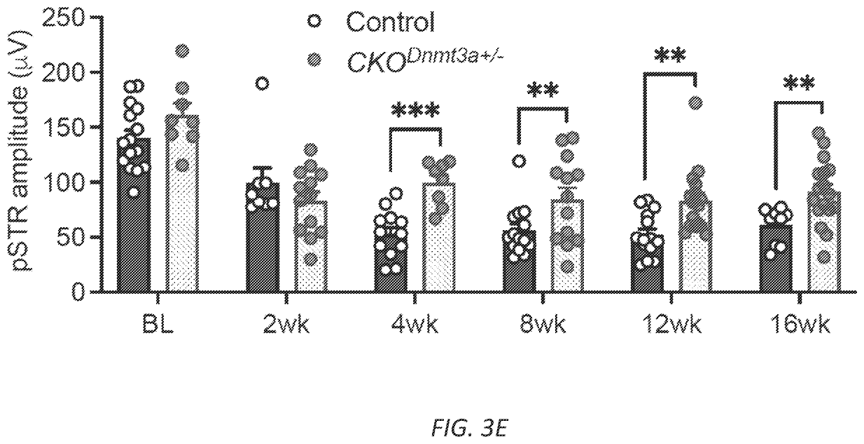

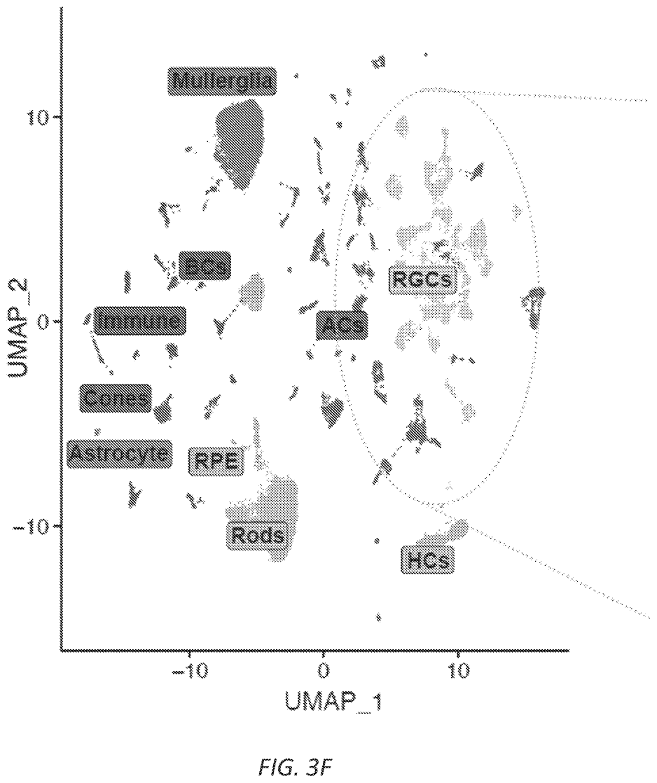

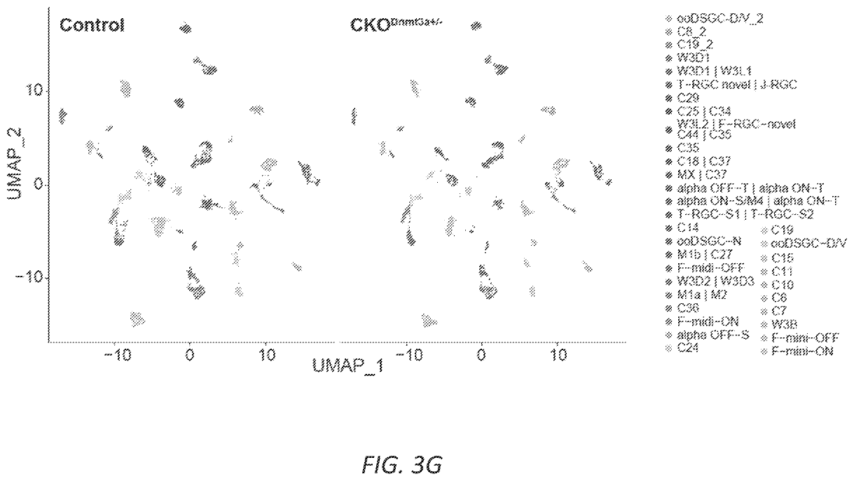

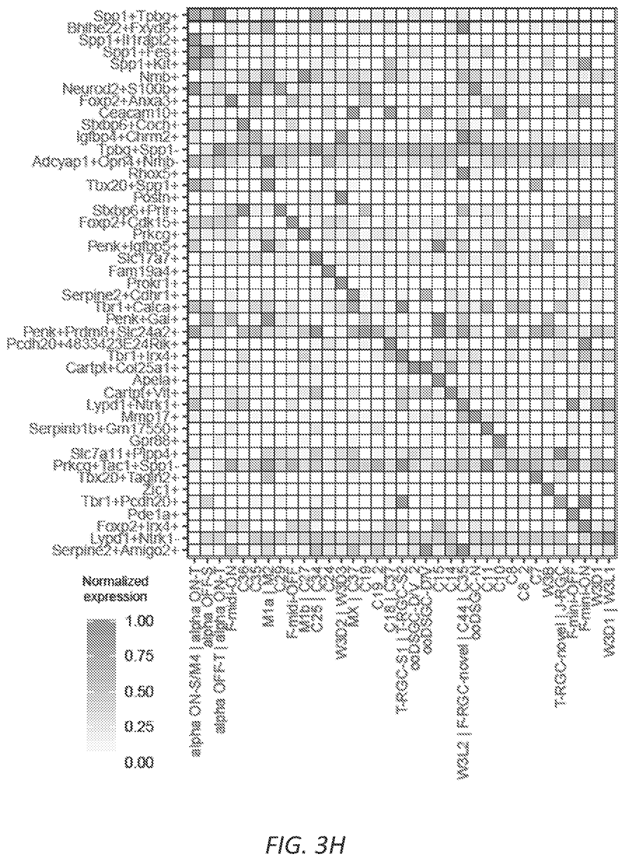

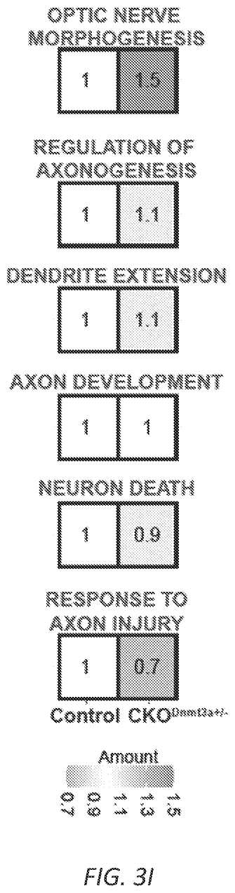

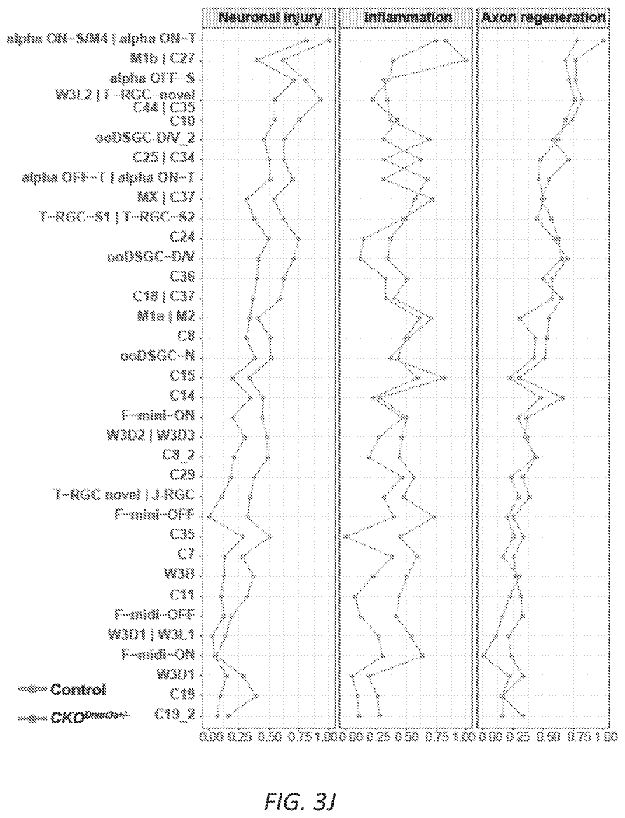

Next, it was tested if the optic nerve reinnervation into the brain targets of adult CKO^{Dnmt3a+/-} mice led to the restoration of RGC function and vision after ONC (FIG. 3A). The mouse visual function was assessed by measuring the optomotor response (OMR), which uses the head tracking behavior of mice to determine their spatial vision (e.g., visual acuity) (Shi et al., 2018). Normal mice without the injury typically exhibited a visual acuity of 0.4-0.5 cycle/degree as measured by OMR. When examined at 2 weeks post ONC, all animals exhibited a complete loss of head tracking behavior in OMR assays, indicative of blindness following nerve injury (FIG. 3B). OMR remained absent in control mice throughout the study period (up to 16 weeks post-lesion). In contrast, ~40% of the CKO^{Dnmt3a+/-} mice started to show a recovery of OMR by 4 weeks post-injury, a time corresponding to the entry of RGC axons into the brain and reinnervating the central visual targets as shown above. The percentage of CKO^{Dnmt3+/-} mice which had regained OMR increased with time and reached ~70% by 16 weeks post-ONC. Quantification of OMR-based visual acuity reached 0.26 cycle/degree, a recovery of more than 50% of the normal value of visual acuity in mice (FIG. 3B).

Additional assays were performed to evaluate the RGC and visual functions. First, in a dark/light preference assay, normal mice spent over 80% of their time in a dark environment. Wildtype mice subjected to ONC showed no preference for a dark environment when examined up to 16 weeks post-injury. In contrast, the CKO^{Dnmt3a+/-} mice with ONC recovered to the same level as observed in uninjured mice by 16 weeks post injury (FIG. 3C), showing a clear indication of a restoration of light perception. Second, the visually evoked potentials (VEPs) in the cortex were tested. Control mice showed an absence of or flat VEP N1 wave (with an infinite delay of N1 latency; FIG. 3D and FIG. 7C, D) up to 16 weeks after nerve crush. Whereas, in parallel with the OMR recovery, ~60% of CKO^{Dnmt3a+/-} mice regained VEP N1 responses at an amplitude to half of its baseline value by 16 weeks post-injury (FIG. 3D; FIG. 7D). Of note, there was a significant delay in N1 latency in CKO^{Dnmt3a+/-} mice after injury compared to the normal uninjured group (FIG. 7C, D), potentially due to immature synaptic reconnection or incomplete myelination of the regenerated axons that should impede the speed of electrical signal transmission from the eye to the brain (Ridder et al., 2006; You et al., 2011). Moreover, significant increase in the amplitude of positive scotopic threshold response (pSTR) (Tang et al., 2020), a readout of the electrical activity of RGCs, was detected in CKO^{Dnmt3a+/-} mice during the period of 4-16 weeks post-injury when compared to control mice (FIG. 3E; FIG. 7E). Together, these results demonstrated the reversal of RGC function and vision loss following axon regeneration in a crush-injured optic nerve in adult mice via a single gene manipulation.

### Example 3: Wide-Scale Transcriptome Shifts by Various RGC Types Towards an Injury Resilient and Regenerative Status

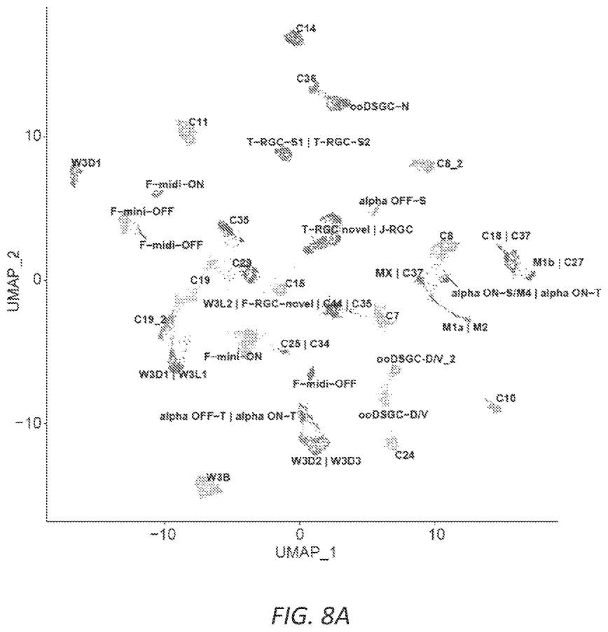

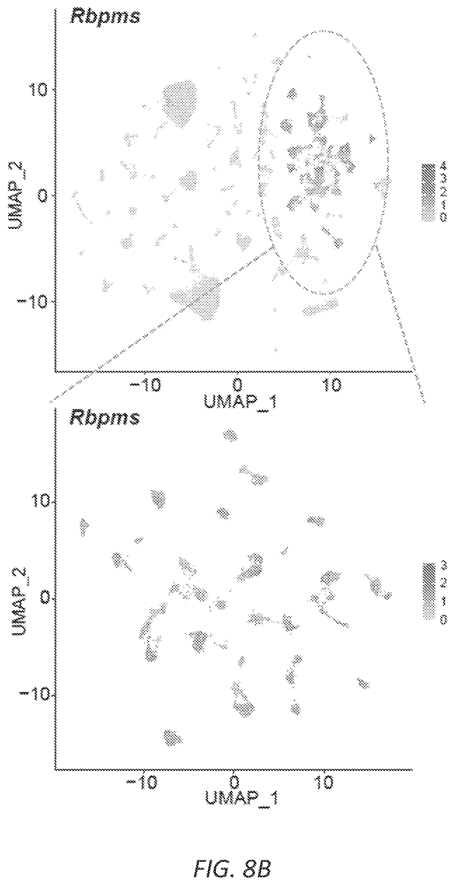

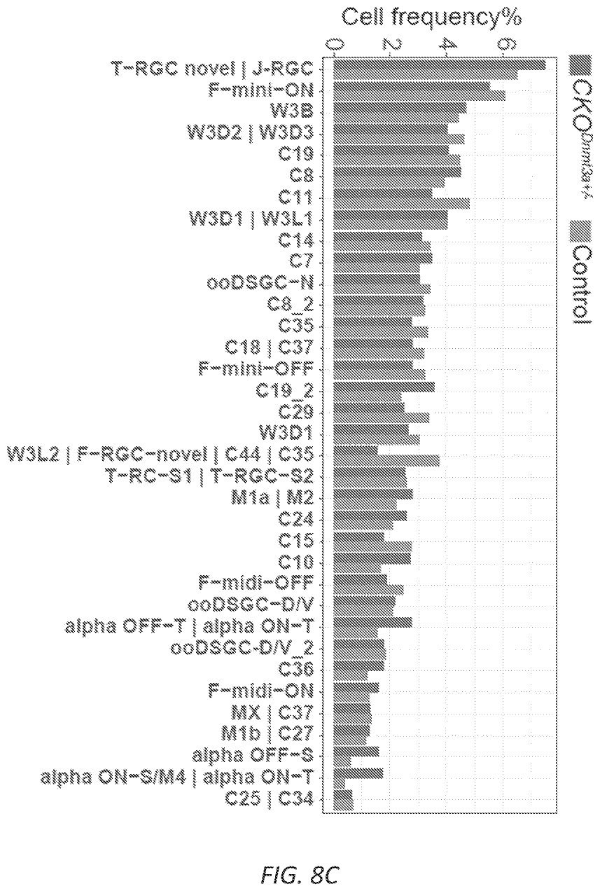

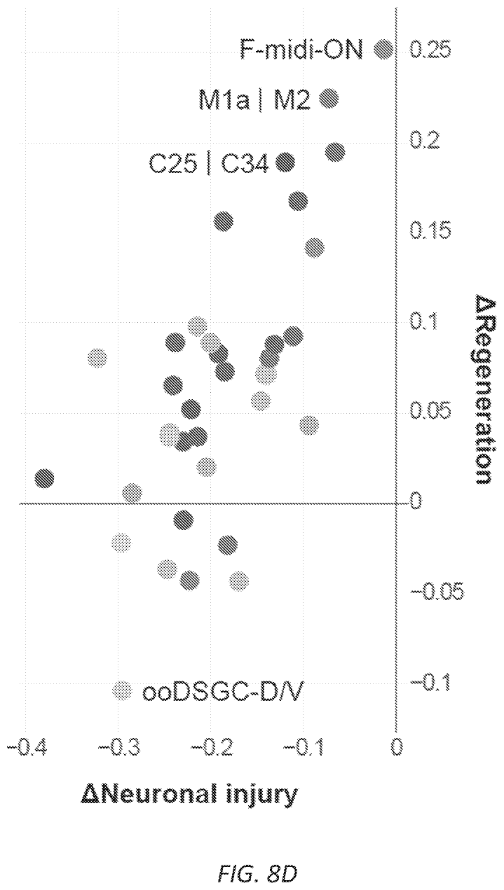

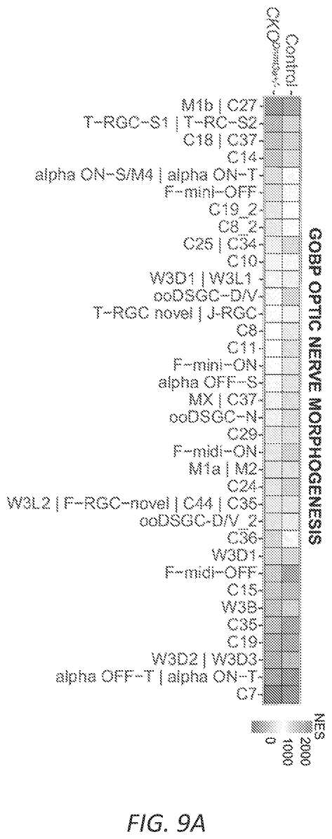

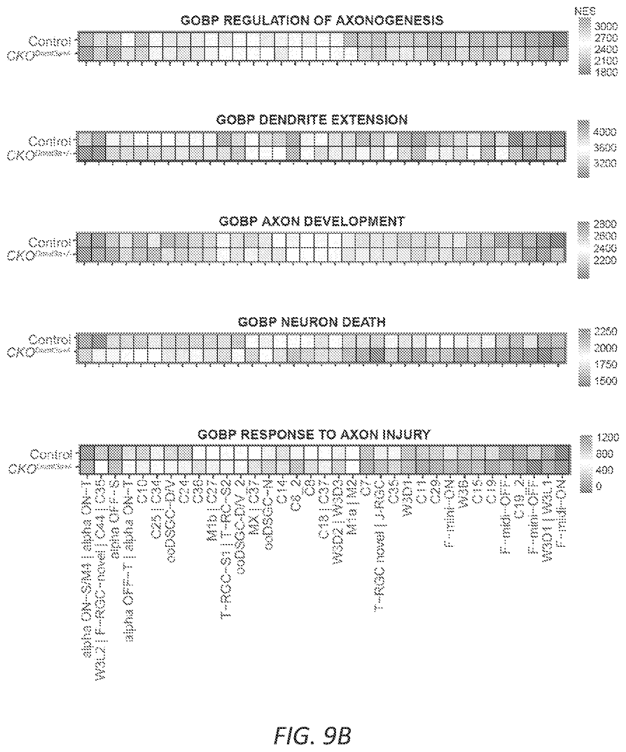

Mouse retina contains 45 RGC types that differ dramatically in their survival and regenerative ability following ONC (Tran et al., 2019), therefore, it is important to understand if DNMT3a deficiency promotes axon regeneration of specific RGC types. To examine if the DNMT3a deficiency promotes axon regeneration of specific RGC types by shifting the RGC landscape or by altering the injury responses (pathophysiology condition) in selective RGC types, the RGC transcriptome was profiled using the single nuclei RNA sequencing. Retinas were collected at 2 days post-injury when few if any RGCs have died, but responses to injury are already apparent (Chen et al., 1997; Jacobi et al., 2022). RGC nuclei were enriched by fluorescence-activated cell sorting (FACS) using a NeuN antibody and profiled by droplet-based snRNA-seq using the 10× platform (Lim et al., 2016). It was examined if the proportions of RGC types was affected by Dnmt3a deficiency. In the retinas of both CKO^{Dnmt3a+/-} and control mice, it was observed that 35 RGC clusters, within which 42 of the 45 reported RGC types were identified based on the list of RGC atlas markers (Tran et al., 2019; Jacobi et al., 2022) (FIG. 3F-H and FIG. 8A-C). More than half of the clusters (24/35) were 1:1 matches with the reported atlas RGC types, while the other 11 each contained 2-4 RGC types (FIG. 3H). The mixture of RGC types within clusters is likely due to the smaller number of RGCs profiled here compared to the earlier report. Virtually, no differences were found in the distribution of RGC clusters between CKO^{Dnmt3a+/-} and control mice (FIG. 3G). Over 90% of RGC clusters showed similar frequencies in CKO^{Dnmt3a+/-} and control mice (FIG. 3F-H and FIG. 8A-C). Notably, the frequencies of three aRGC subsets (αON-S/M4|αON-T, αOFF-S and αOFF-T|αON-T), which are the rarest and the most vulnerable and regenerative RGC types (Lim et al., 2016; Duan et al., 2015), were slightly increased in CKO^{Dnmt3a+/-} mice compared to controls. They collectively represented ~6% of the total RGC population in CKO^{Dnmt3a+/-} mice compared to <3% in control mice (FIG. 8C). Collectively, DNMT3a deficiency does not alter the distribution of retinal cell and RGC profiles or drastically changes the frequencies of the vast majority of RGC types except it somewhat increases the occurrence of rare αRGC subsets.

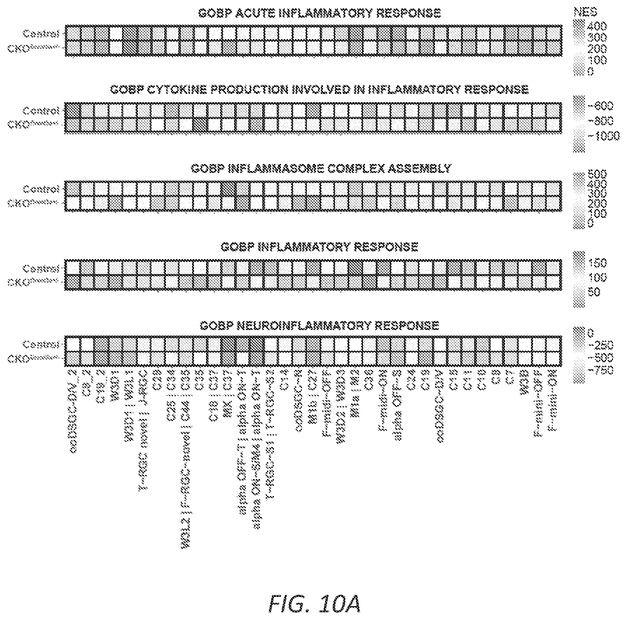

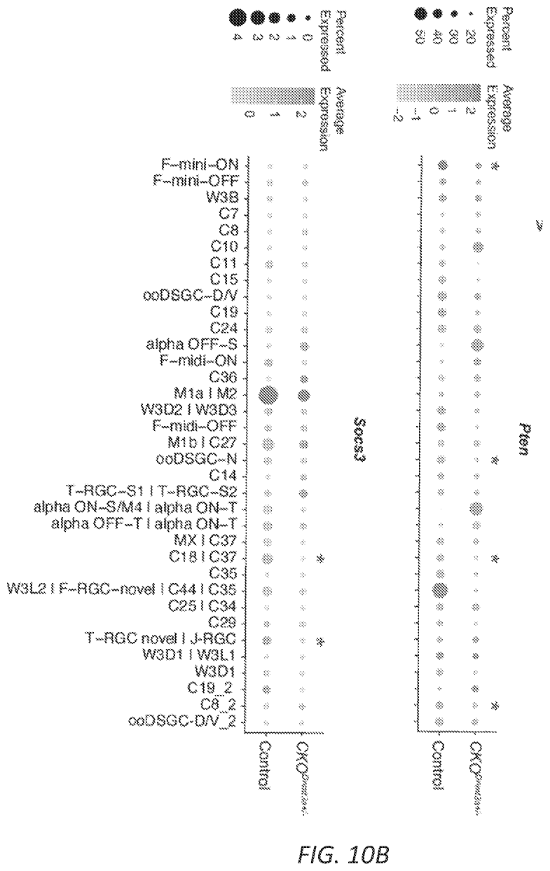

Based on these findings, differences in gene expression changes were assessed after ONC between the control and the CKO^{Dnmt3a+/-} RGCs. In line with the robust optic nerve regeneration, upregulation of the nerve growth-related pathways was observed as classified by Gene Ontology (GO) terms, including optic nerve morphogenesis, axonogenesis and dendrite extension (FIG. 3I). In contrast, genes associated with neuron death, axon injury response, and neuroinflammation signals classified by GO terms, were downregulated compared to controls, indicating enhanced resilience to injury. By combining the normalized enrichment scores (NES) of injury response- and growth-regulated gene categories, respectively, in each individual RGC clusters, a consistent pattern of gene expression changes was noted across a broad spectrum of RGC types (FIG. 3J and FIG. 8D, 4A, B, 5A), indicating cell type-independent transformation into growth-promoting and injury-resilient states. Surprisingly, comprehensive downregulation of Pten expression, a well-established endogenous inhibitor of axon regeneration which, if deleted promotes selective axon regeneration of αRGCs (Rheaume et al., 2023: Sun et al., 2011), was not observed in the majority of CKO^{Dnmt3a+/-} RGC types, except in four clusters (FIG. 10B). Moreover, only two CKO^{Dnmt3a+/-} RGC clusters displayed significantly decreased expression of Socs3, another well-known axon regeneration inhibitor (Jacobi et al., 2022: Sun et al., 2011), when compared to the controls. These findings suggest that CKO^{Dnmt3a+/-} mice can switch on a Pten- or SOCS3-independent axon growth mechanism. Contrary to promoting nerve regeneration in selective RGC types, DNMT3a deficiency triggered a broad-spectrum RGC-transcriptome shift toward a gene profile associated with axon regeneration in nearly all RGC types, including those previously shown to be susceptible to injury (e.g., C35. C25/C34) and those relatively resilient to injury (e.g., aRGCs, M1-RGCs) (Tran et al., 2019). Thus, Dnmt3a deficiency results in wide-scale change of transcriptome profiles in various RGC types toward injury-resilient, less inflammatory, and pro-regenerative states after ONC.

### Example 4: Multifaceted Induction of Axon Regeneration Machinery in Dnmt3a Deficient RGCs

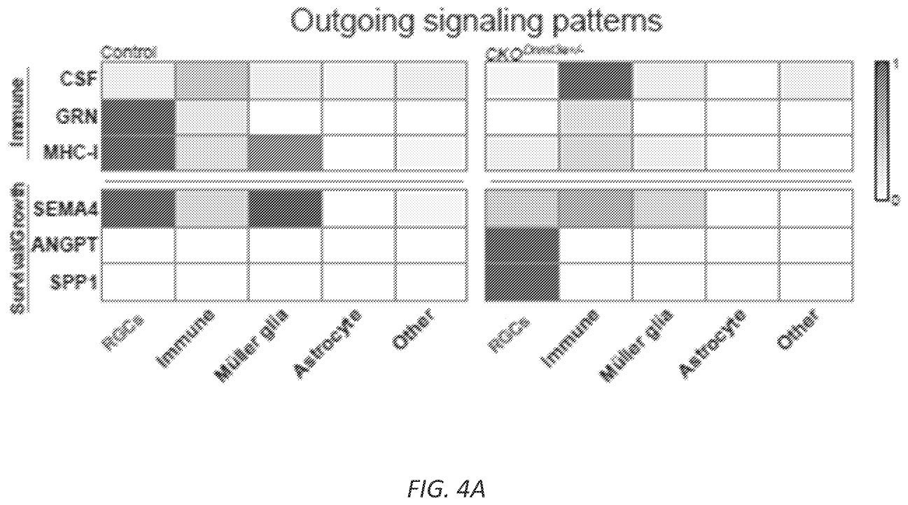

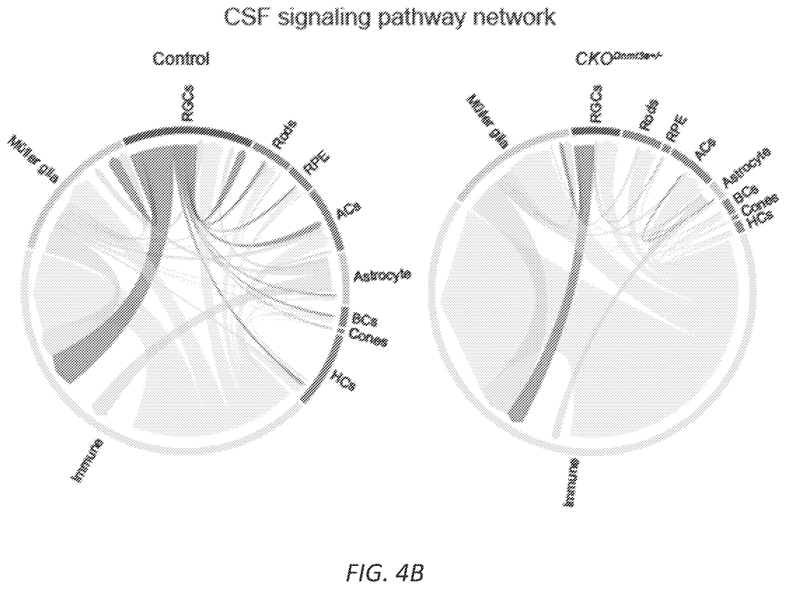

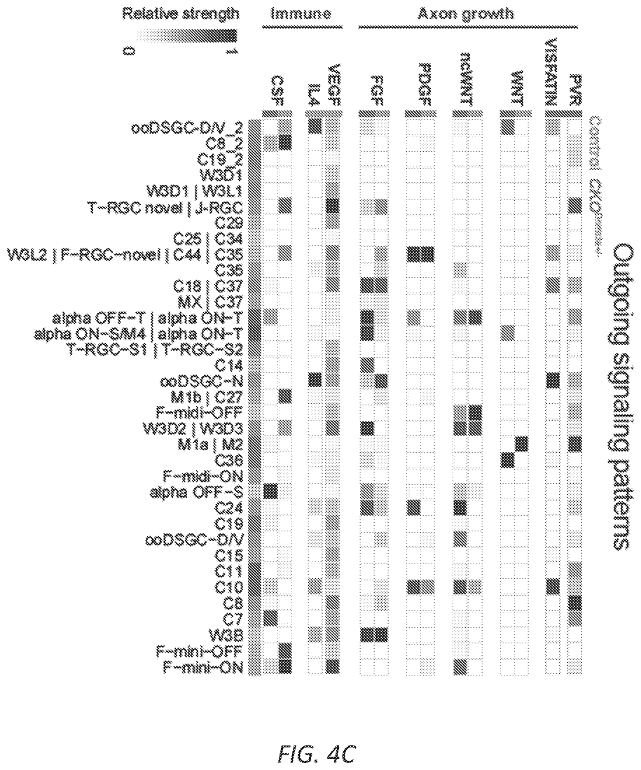

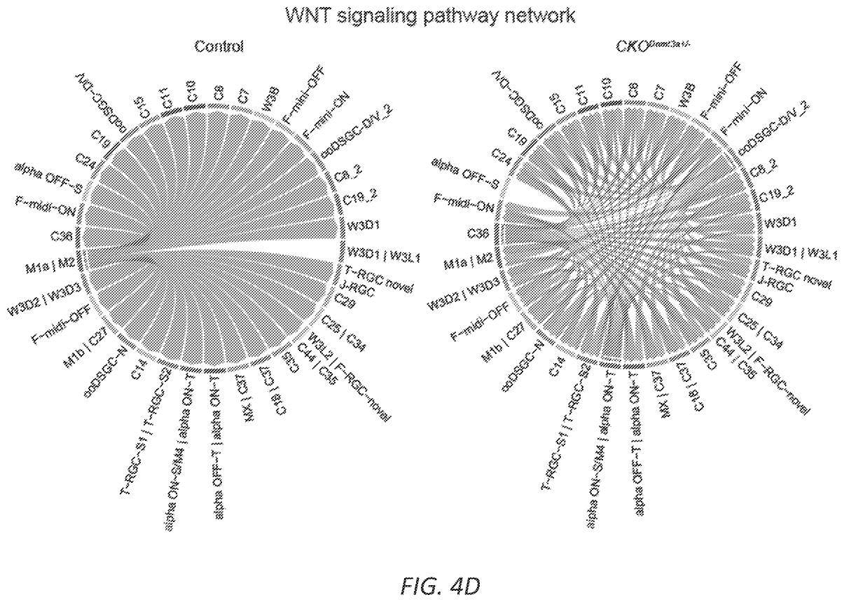

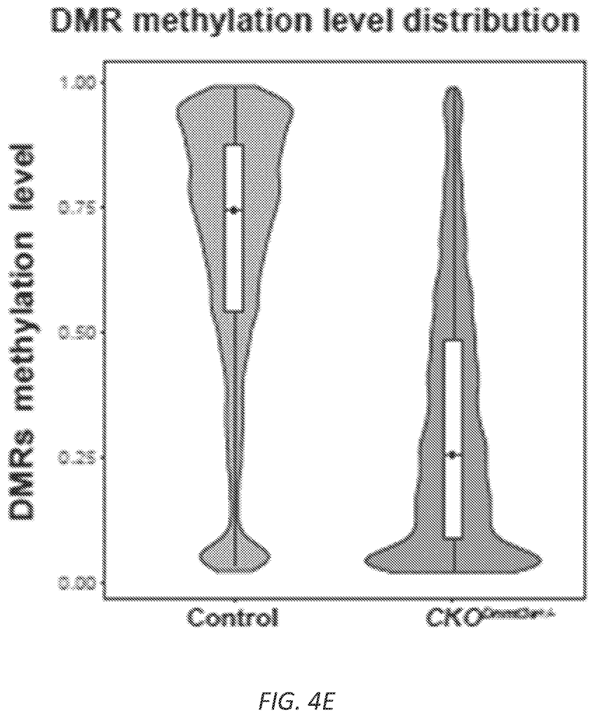

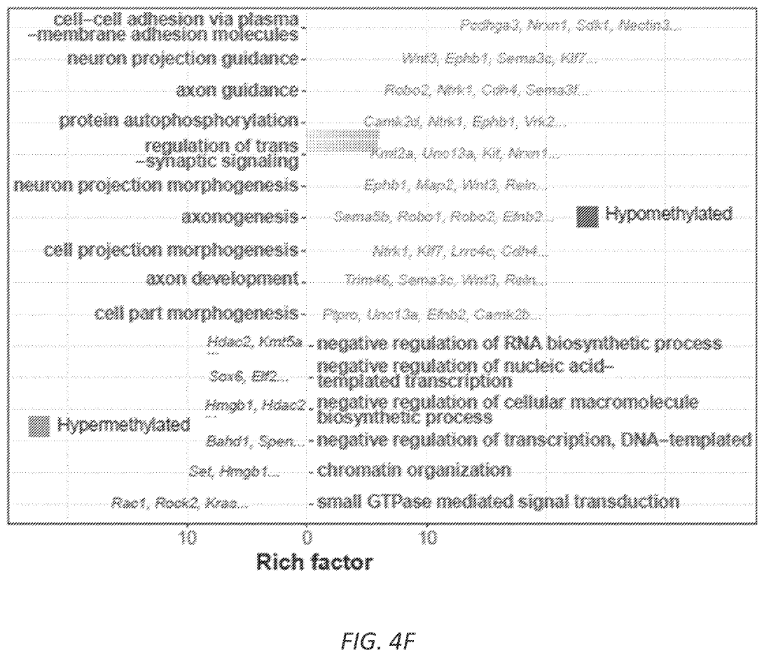

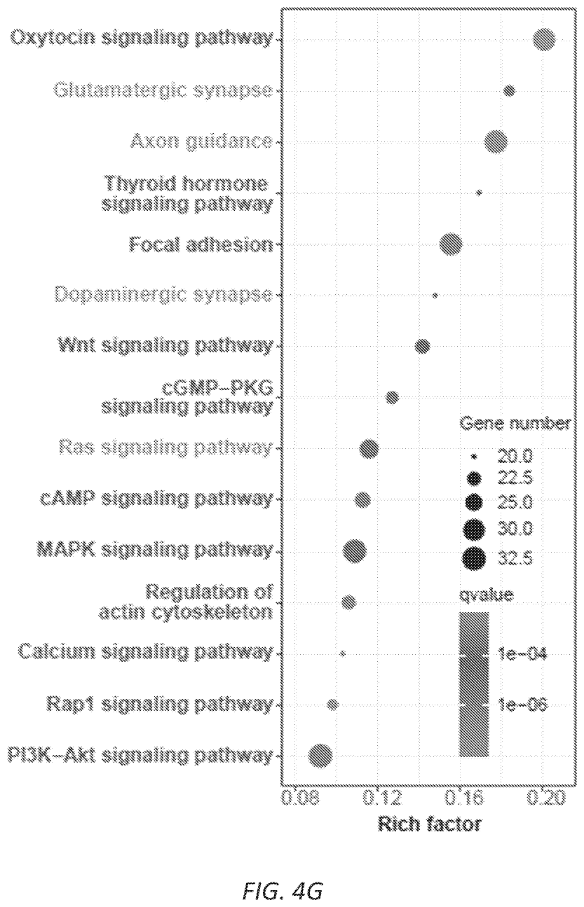

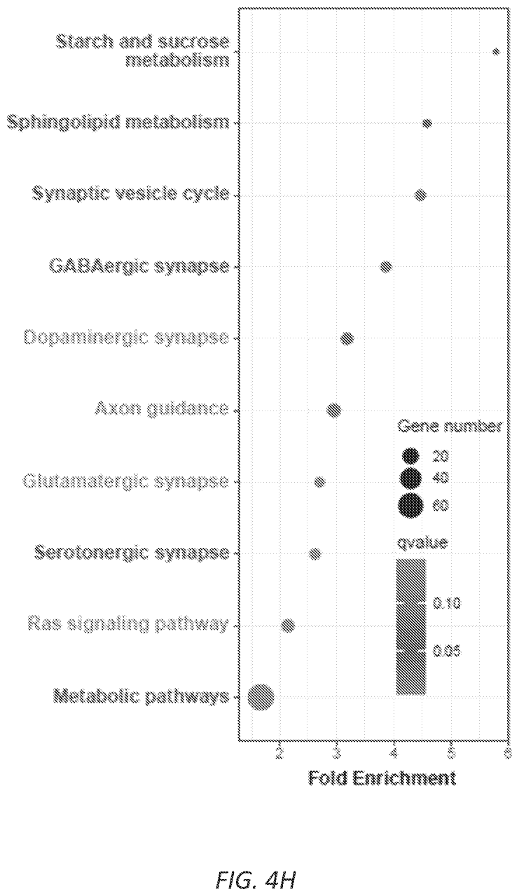

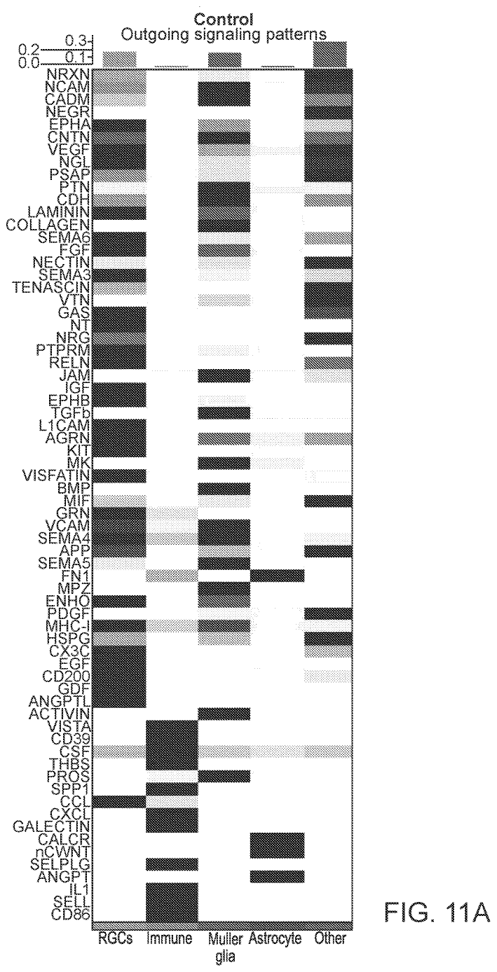

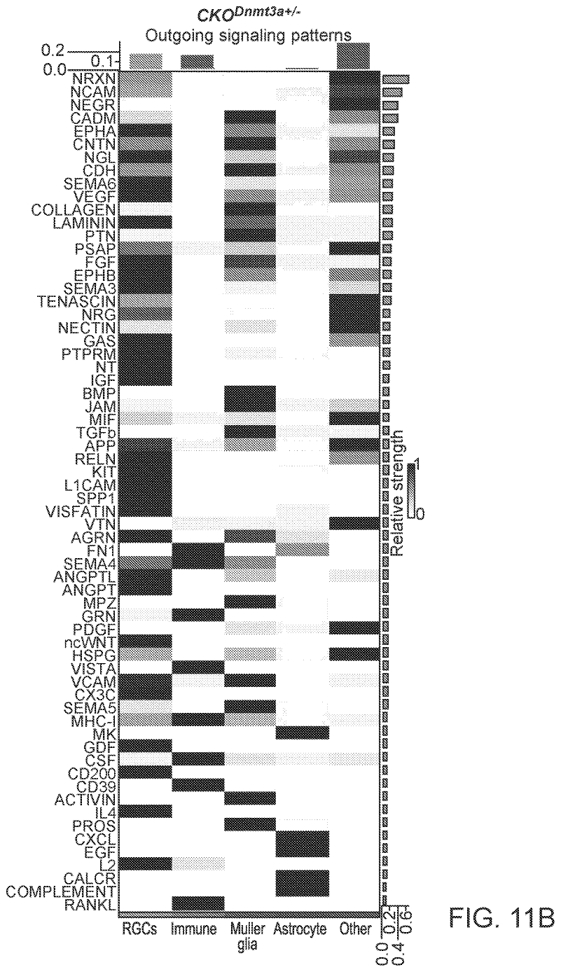

The inability of RGC axons to regenerate is believed to stem from a complex interplay of intrinsic factors and environmental cues, with CNS glial cells releasing axon growth-inhibitory signals and posing a formidable barrier to axon regeneration (Varadarajan et al., 2022). To gain deeper insights into the signaling mechanisms through which RGC-specific Dnmt3a deficiency disinhibits the development of glial barrier to axonal regrowth, CellChat analysis, a bioinformatic algorithm designed to predict and decipher intercellular cues exchanged by various cell types (Jin et al., 2021) was used. Given that DNMT3a dysfunction in CKO^{Dnmt3a+/-} mice is RGC-specific, the focus was placed on the outgoing signals from the RGCs to other cell types identified in our snRNA-seq (FIG. 3F). As shown above, CellChat analysis consistently predicted significant downregulation of RGC-derived inflammatory signals in the CKO^{Dnmt3a-/-} mice compared to the controls, primarily associated with three immune activator (FIG. 4A and FIG. 11A, B): colony stimulating factor (CSF) (Tang et al., 2020), granulin (GRN) (Park et al., 2011), and major histocompatibility complex class 1 (MHC-1) (Rao et al., 1993). Notably, the chord diagram showed that in control mice, CSF originating from the RGCs signaled to multiple other retinal cell classes, especially immune cells, whereas this RGC signal was drastically diminished in the CKO^{Dnmt3a-/-} mice (FIG. 4B). DNMT3a deficiency also led to downregulation of axon growth-inhibitory signals, such as Semaphorin 4 (SEMA4) (Carulli et al., 2021), and upregulation of synapse and axon growth-promoting signals, such as angiopoietin (ANGPT) (Zhou et al., 2019; Luck et al., 2021), and secreted phosphoprotein 1/osteopontin (SPP1) (Li et al., 2022) (FIG. 4A). Therefore, RGC-specific DNMT3a deficiency not only induces an intrinsic pro-regenerative program within RGCs but also enables a permissive environment by sending decreased inflammatory signals and increased growth-promoting signals.

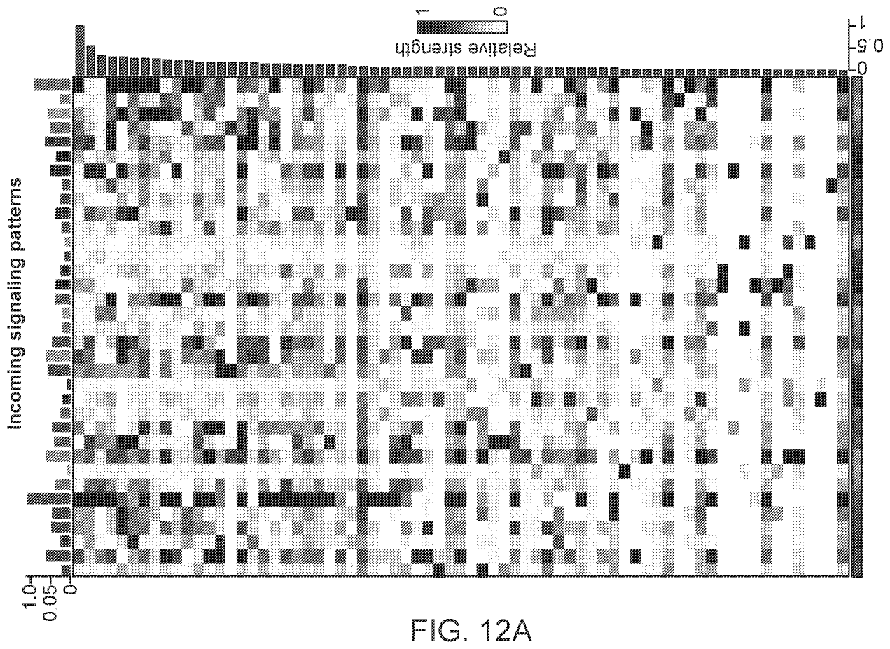

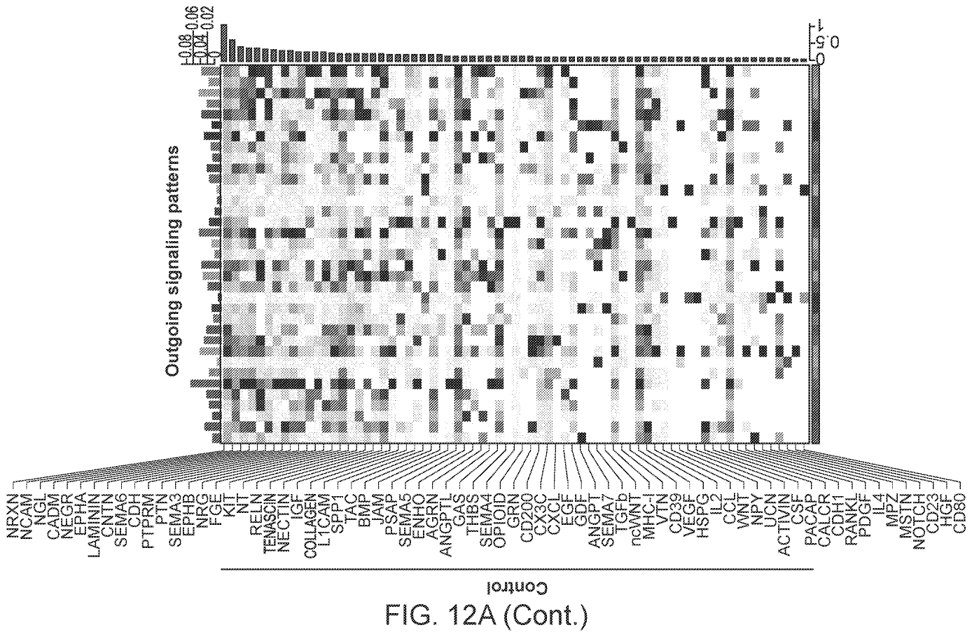

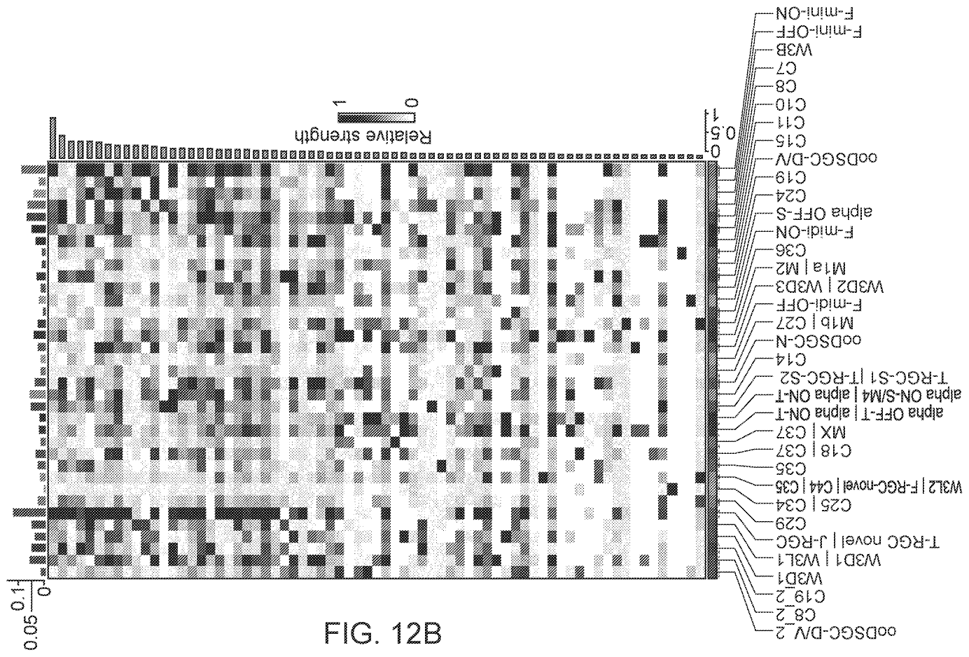

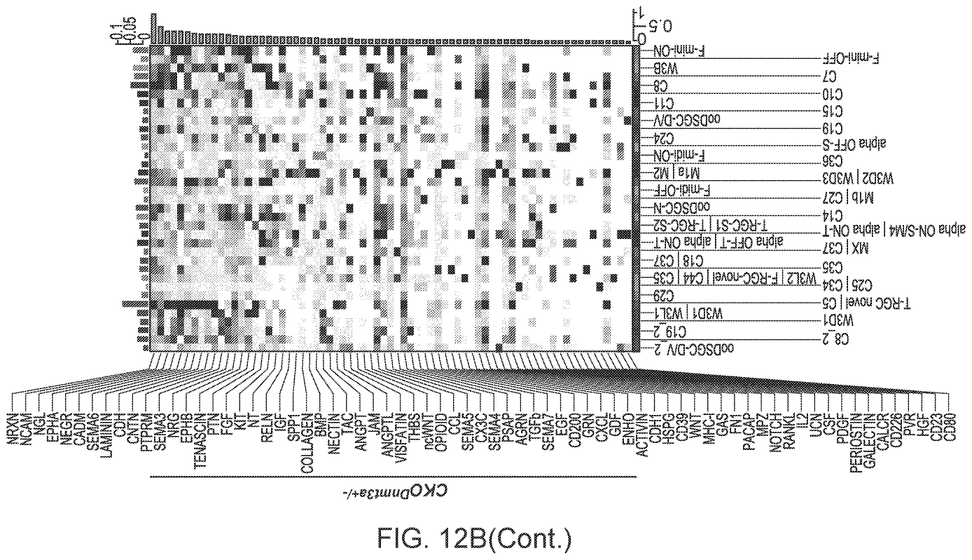

To determine how DNMT3a deficiency alters the communication among RGC types, CellChat signaling was compared among the RGCs in CKO^{Dnmt3a+/-} and the control mice. The results indicated the up-regulation of classical Wnt (Garcia et al., 2018) and non-canonical Wnt (Musada et al., 2022), PDGF and FGF signaling (Johnson et al., 2014) by many RGC types in the CKO^{Dnmt3a+/-} mice compared to the controls (FIG. 4C and FIG. 12A, B). The chord diagram of the WNT communication indicated that intrinsically photosensitive RGC types M1a and M2 were the sole major source of WNT signal in controls, while in the CKO^{Dnmt3a+/-} mice, multiple RGC types upregulated outgoing WNT signaling to other RGCs (FIG. 4D). The CKO^{Dnmt3a+/-} RGCs also exhibited distinctive expression of poliovirus receptor (PVR) (Ikeda et al., 2004; Kajita et al., 2007) and visfatin (Erfani et al., 2015; Erfani et al., 2015), two growth and survival-related genes that were absent in the control RGCs. Conversely, the growth factors with pro-inflammatory properties, such as vascular endothelial growth factor (VEGF) (Carmeliet et al., 2000) and interleukin 4 (Milner et al., 2010; Rakyan et al., 2011), were highly expressed only in the control RGCs but not in the CKO^{Dnmt3a+/-} RGCs (FIG. 4C). These results strengthened the conclusion that RGC deficiency of DNMT3a contributes to distinctive pro-regenerative intercellular signaling and suppressed inflammatory responses.

### Example 5: DNA Methylation of Axon Growth Gene Networks Regulated by Dnmt3a

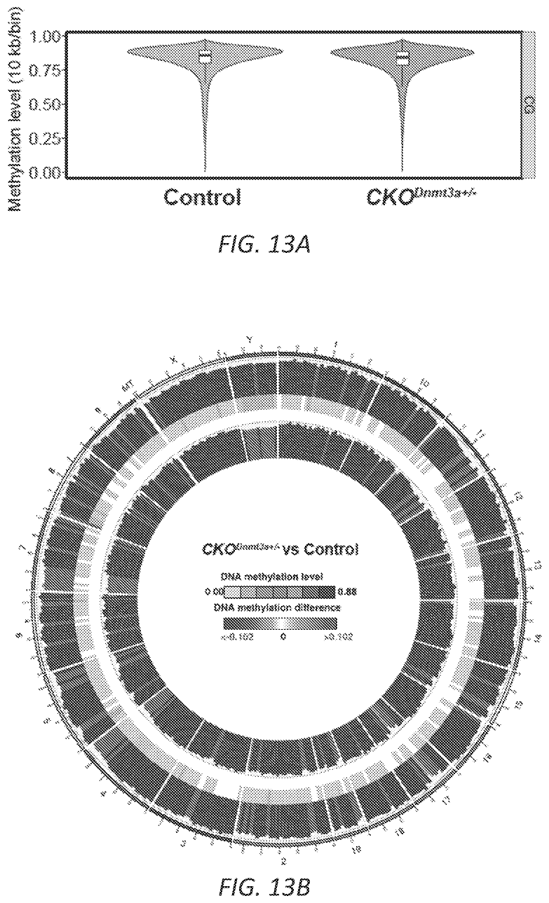

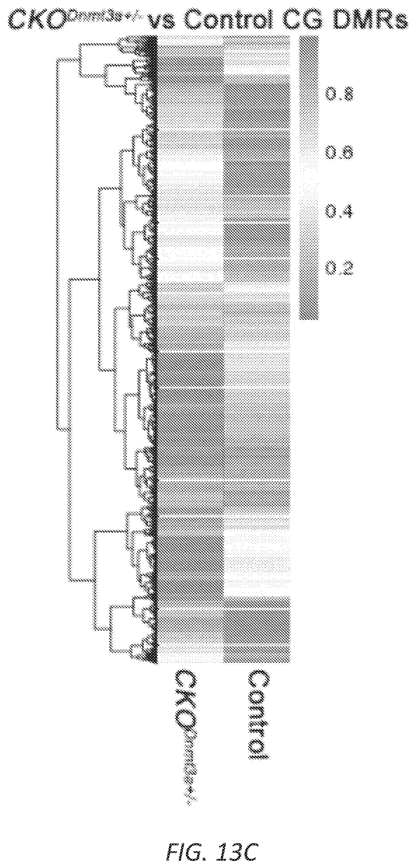

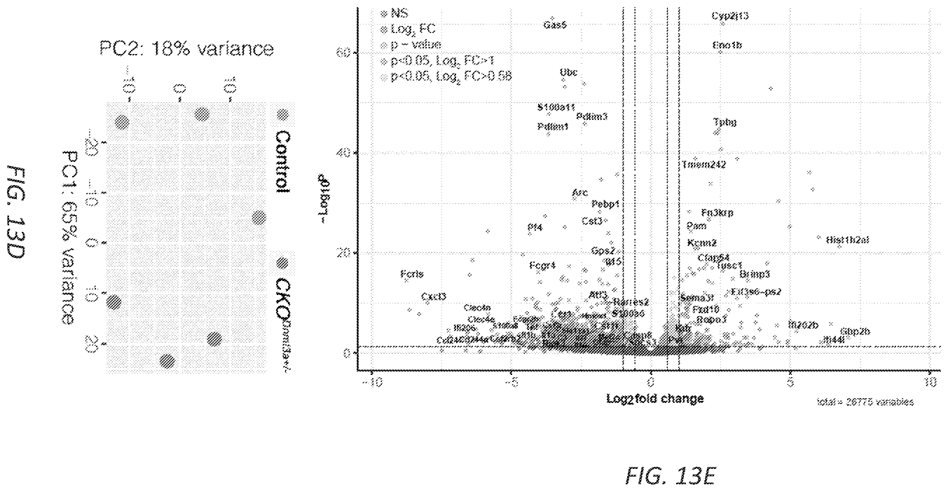

DNMT mediates DNA methylation to serve biological functions. To investigate how DNMT3a deficiency altered the landscape of DNA methylation in RGCs, a whole genome bisulfite sequencing (WGBS) was conducted for the RGCs isolated 2 days post-ONC. Compared to the controls, the CKO^{Dnmt3a+/-} RGCs showed lower CG methylation levels specifically in the differentially methylated regions (DMRs) (FIG. 4E), which genomic regions showed different methylation status across cell types or individuals thus were regarded as possible functional regions involved in gene transcriptional regulation (Rakyan et al., 2011). Minimal alteration in genome-wide DNA methylation level was detected in the CKO^{Dnmt3a+/-} RGCs (FIG. 13A, B). DMRs WGBS disclosed 4,836 CG DMRs, including 4,004 hypo-DMRs and 832 hyper-DMRs (FIG. 13C). Annotation of hypomethylated DMR-related genes identified 48 biological processes. Consistent with the observed transcriptomic shifts toward a pro-regenerative state as shown above in the CKO^{Dnmt3a+/-} RGCs, 14 of the hypomethylated pathways were linked to axon growth, including axon guidance, axonogenesis, and axon development (e.g., Wnt3, Klf7, Map2) (Carulli et al., 2021; Patel et al., 2017; Plachez et al., 2008) and 6 were related to synaptic process (e.g., UNC13a and Nrxn1) (Sudhof et al., 2008; Yang et al., 2011) (FIG. 4F). KEGG pathway analysis in total DMR-related genes again depicted distinctive enrichment in pathways related to nerve growth and synaptic process that includes key signaling events such as Wnt, Ras, and cAMP, axon guidance, and dopaminergic and glutamatergic synapse (FIG. 4G). The data establish that NDMT3a directly regulates the methylation of gene networks that control axon growth and synaptic process.

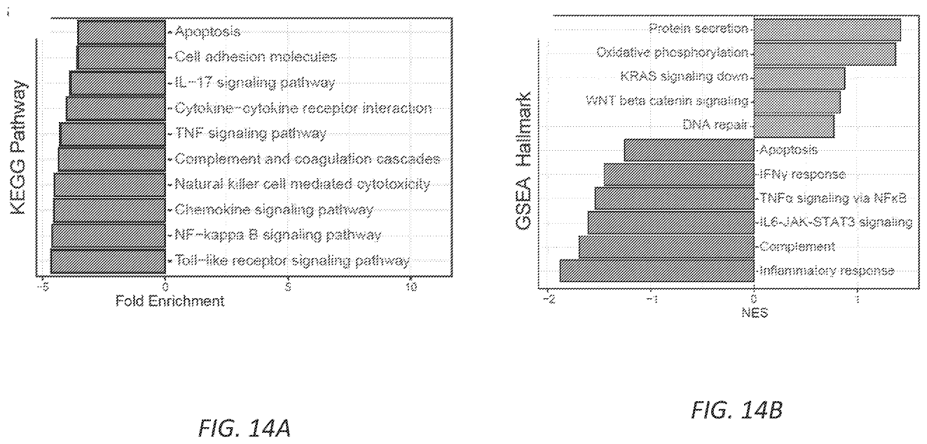

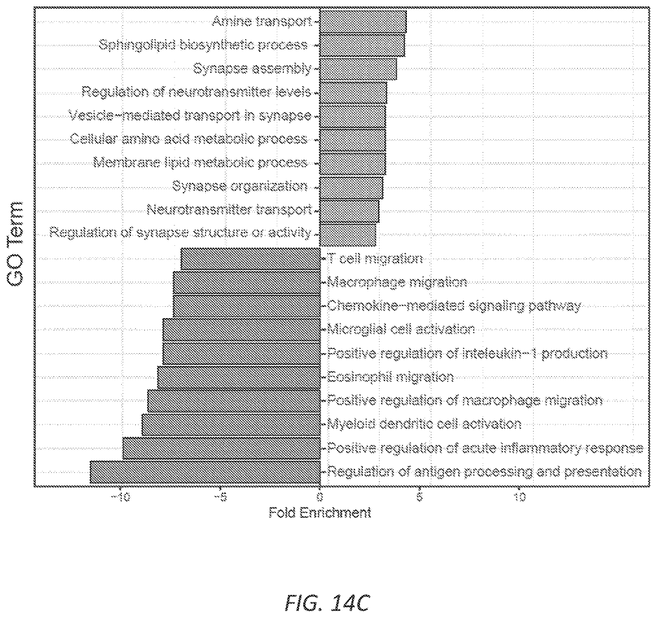

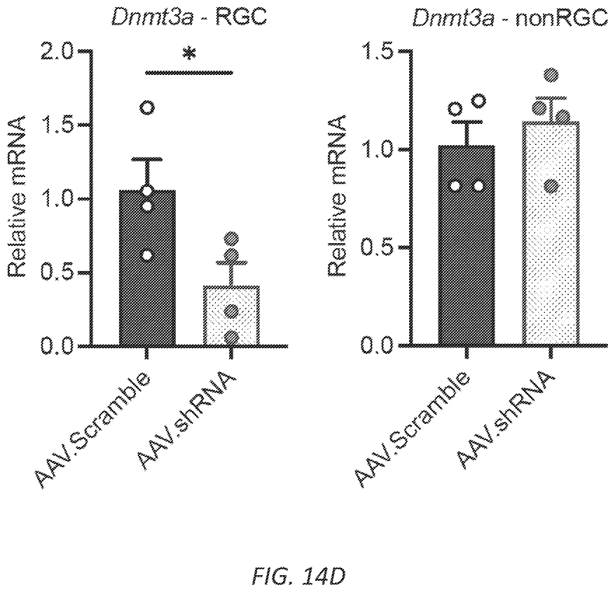

To validate that hypomethylation of genes controlling the axon growth and synaptic process in DNMT3a-deficient mice results in correspondent gene expression changes, comprehensive transcriptome profiling was conducted by a bulk RNA-seq on RGCs isolated two days after ONC. Principal component analysis clearly separated the CKO^{Dnmt3a+/-} RGCs from the controls (FIG. 13D). Differentially expressed (DEGs) identified 1,459 genes (Fold change>1.5; padj<0.05) between CKO^{Dnmt3a+/-} and control RGCs (FIG. 13E). KEGG analyses revealed alignment between upregulated gene pathways in the CKO^{Dnmt3a+/-} RGCs with those found to be hypomethylated above, including synaptic signaling and axon guidance (FIG. 4H). On the other hand, downregulated DEGs in the CKO^{Dnmt3a+/-} RGCs were primarily involved in cell death and immune regulation, such as apoptosis, TNFα and chemokine/cytokine signaling (FIG. 14A). GSEA and GO enrichment studies confirmed the results of KEGG analysis, showing downregulation of immune response (e.g., T cell and macrophage migration, chemokine-mediated signaling, and microglial activation) and inflammatory gene pathways (e.g., IFNγ, TNFα, and IL6-JAK-STAT3 signaling) (Levkovitch-Verbin et al., 2015) with concurrent upregulation of key events regulating axon growth (e.g., Wnt/β-catenin) and synaptic assembling and transport (FIG. 14B, C). This result was congruent with the prediction of the CellChat analysis.

The gene expression pattern changes detected by the bulk RNA-seq in the CKO^{Dnmt3a+/-} and the control mice was verified by qPCR. The CKO^{Dnmt3a+/-} RGCs isolated at 2 days post-injury showed consistent significant differences in the expression of the aforementioned gene pathways, including the upregulation of axon growth-related genes (e.g., mTOR, Spp1, Gap43) and downregulation of growth inhibitors (e.g. Atf3) as well as cell apoptotic and inflammatory genes (e.g., Gas5, Casp3, Casp8, Ripk1) (Varadarajan et al., 2022; Jacobi et al., 2022: Levkovitch-Verbin et al., 2015; Han et al., 2022) compared to the control RGCs (FIG. 41). Interestingly, these differences were only detected in RGCs isolated post ONC. RGCs taken from the uninjured CKO^{Dnmt3a+/-} mice showed no significant differences in the expression of any of these genes compared to the RGCs from the uninjured control mice (FIG. 4I). The data suggest that the loss of DNMT3a driven by Vglut2-Cre has little impact on gene expression of normal RGCs but rather results in reprogramming of RGCs' injury responses toward an injury-resilient and pro-regenerative status post-ONC. Together, these studies demonstrate that Dnmt3a deficiency reshaped the injury-induced transcriptomic and intercellular communication landscape of RGCs, contributing to the functional regeneration of the optic nerve.

### Example 6: Therapeutic Inhibition of Dnmt3a Rescued Visual Function in Adult Wildtype Mice after Optic Nerve Crush (ONC)

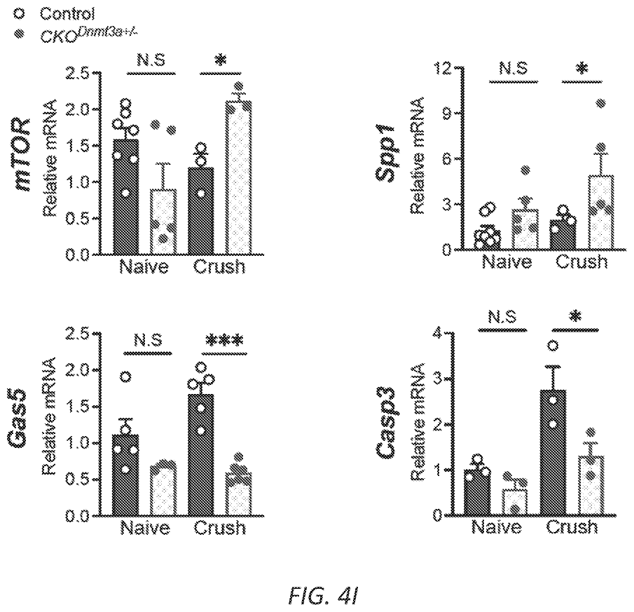

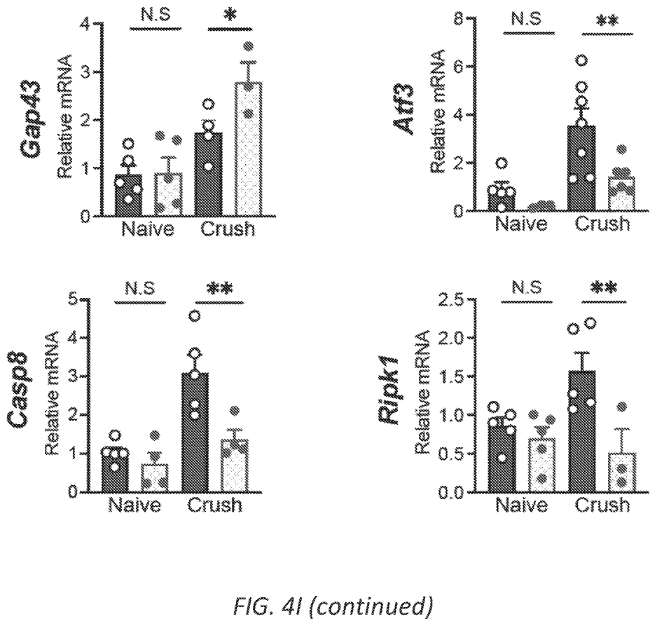

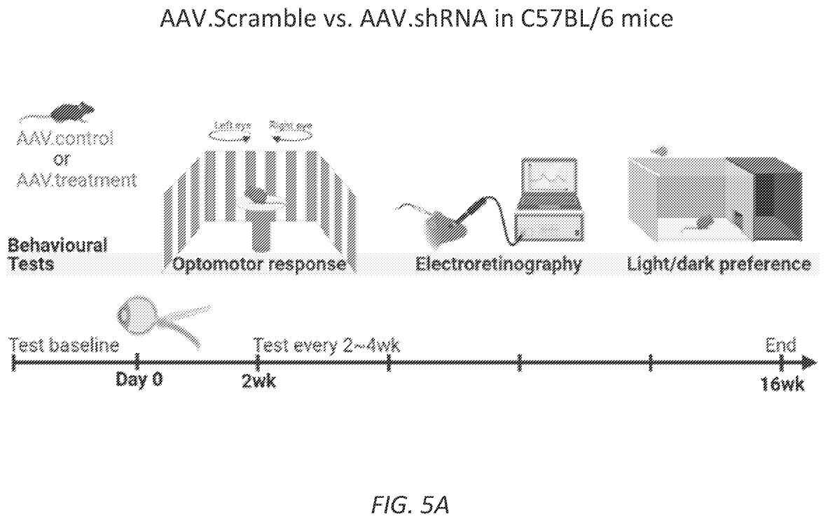

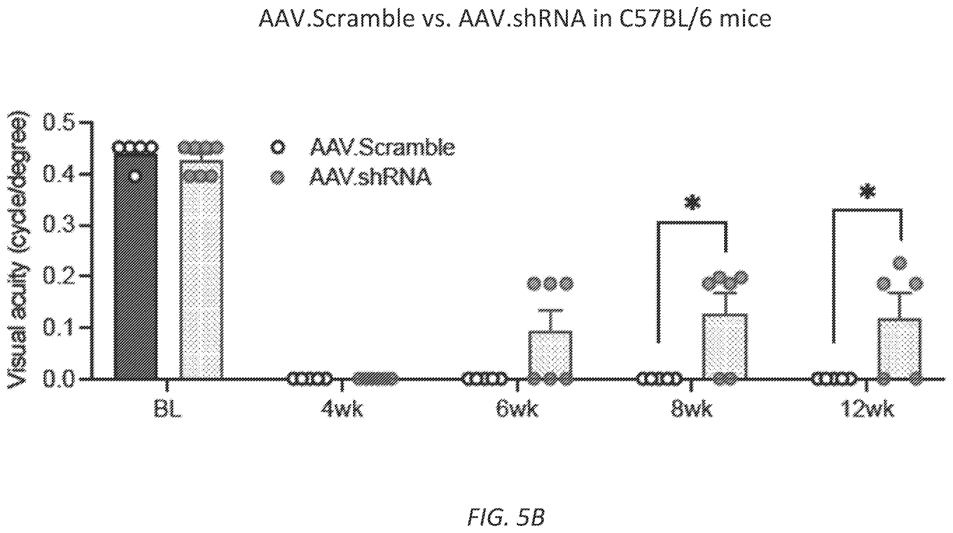

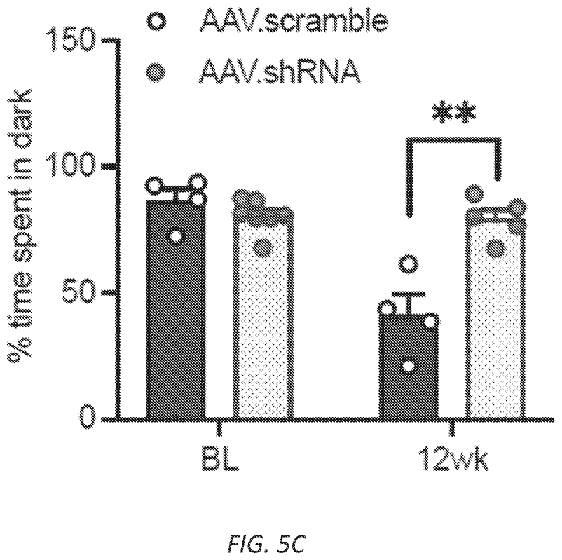

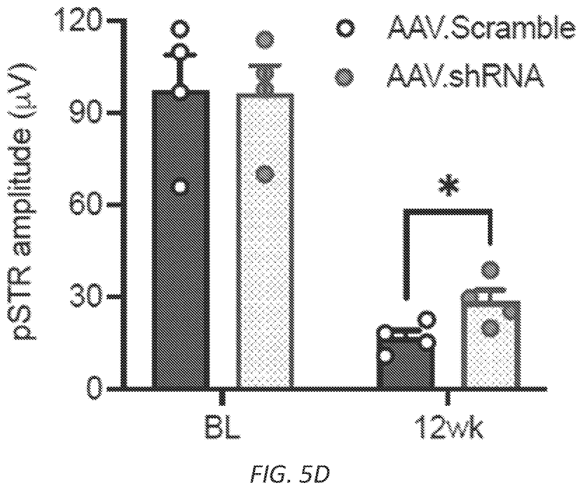

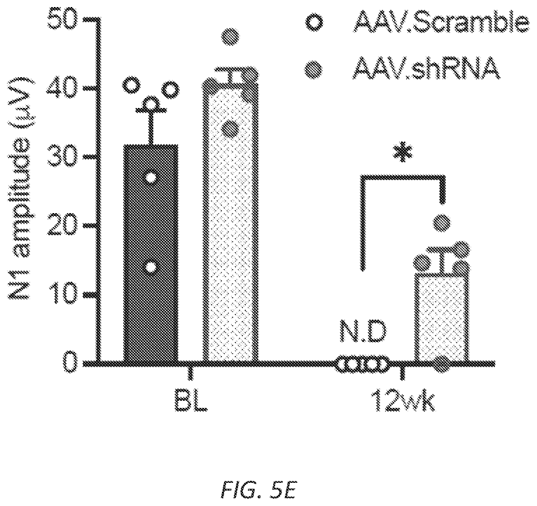

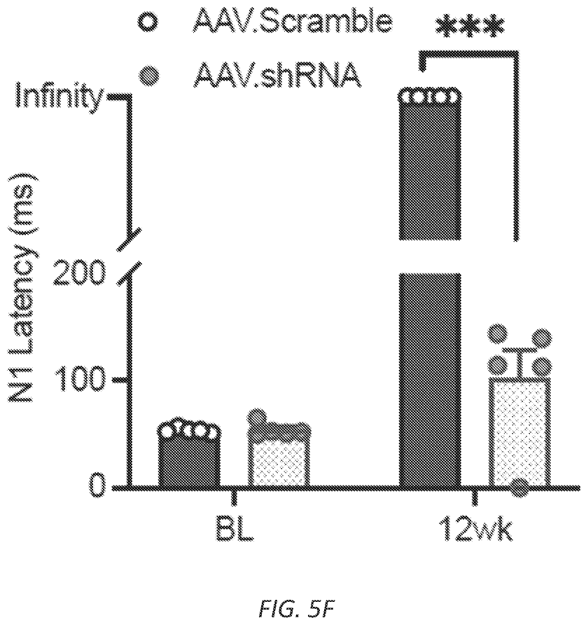

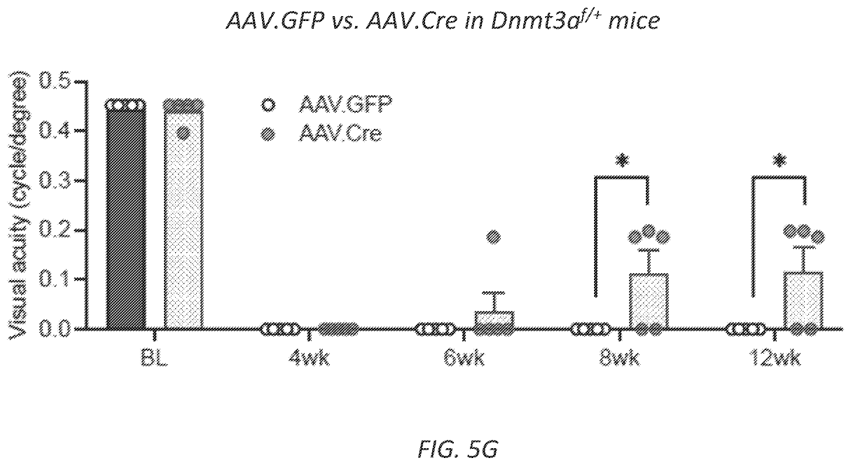

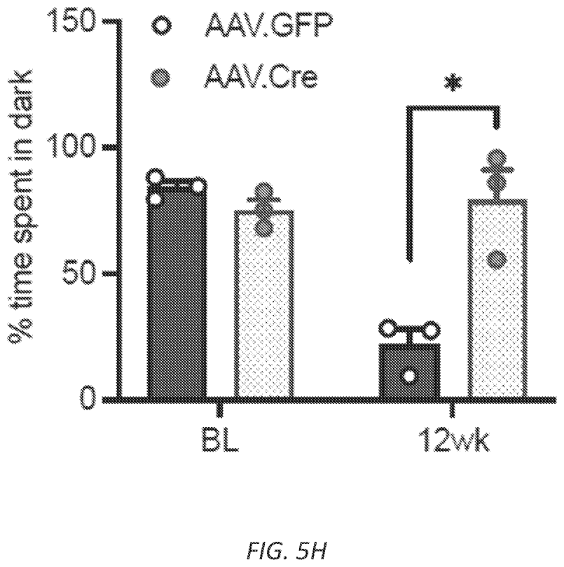

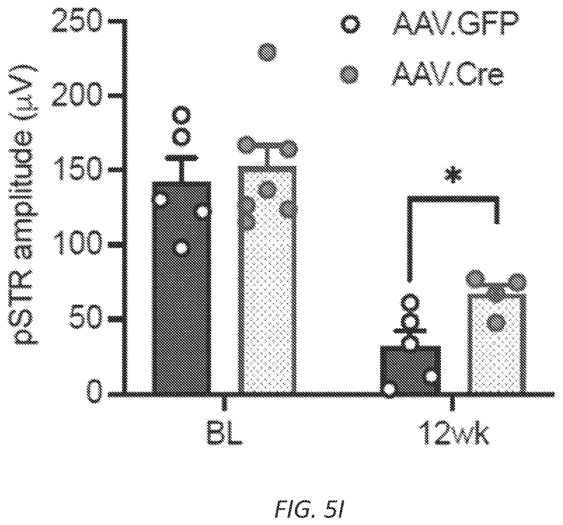

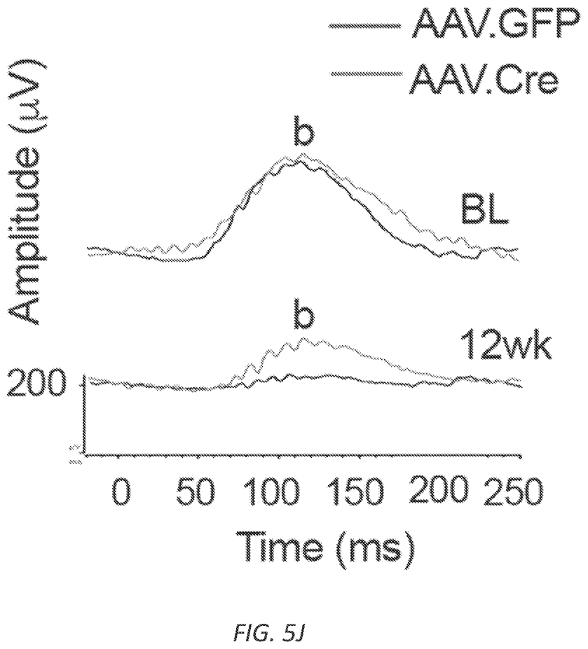

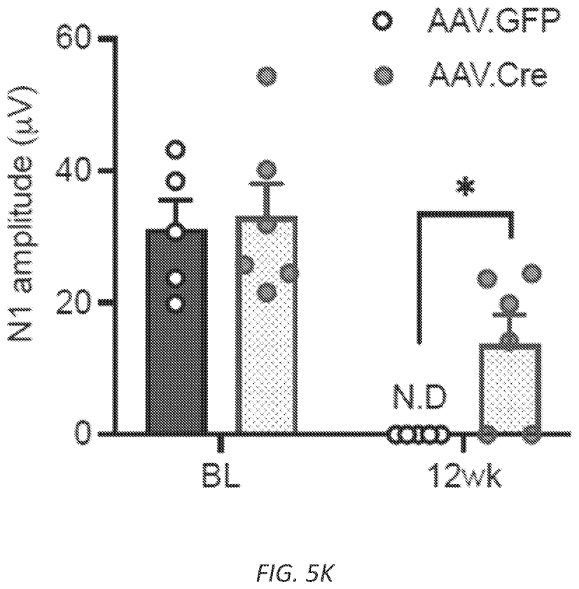

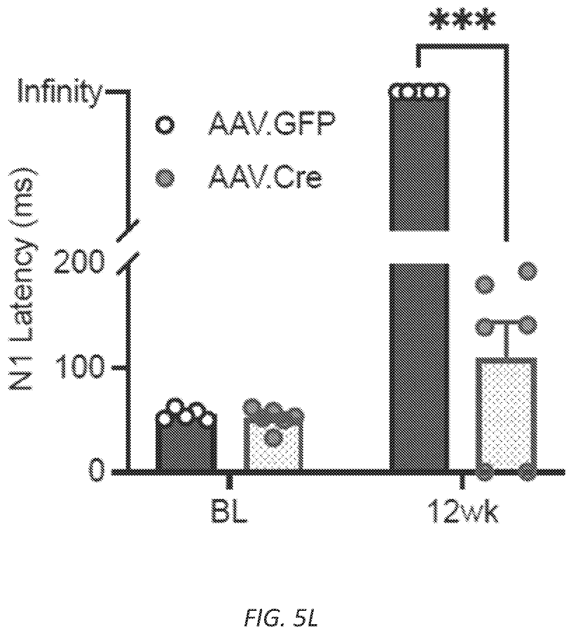

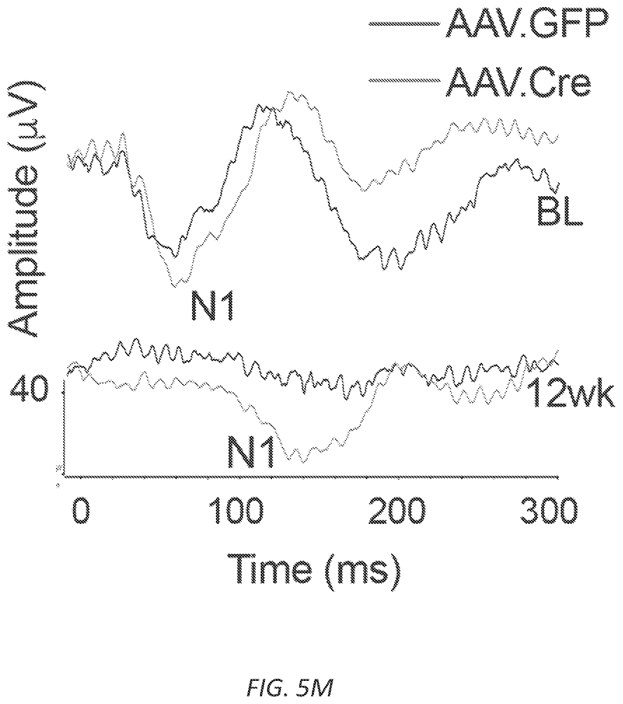

To assess the therapeutic potential of Dnmt3a inhibition, AAV treatment experiments were conducted in adult mice after ONC (FIG. 5A). To downregulate the expression of DNMT3a, AAV-shRNA was injected intravitreally into adult wildtype mice or AAV-Cre was injected into mice carrying heterozygous floxed Dnmt3a allele (fl/+) immediately after ONC. Detection of GFP expression revealed wide-spread AAV-infection in RGCs, and qPCR analysis confirmed the downregulation of the Dnmt3a mRNA levels in RGCs of eyes injected with AAV-shRNA or AAV-Cre compared to AAV-scrambled RNA or AAV-GFP injected eyes (FIG. 14D, E). Tracking of the spatial vision with OMR at multiple timepoints post-injury indicated partial recovery of visual acuity in over 50% of the mice that received AAV-shRNA treatment starting at 6 weeks post-injury (FIG. 5A, B), a two-week delay of recovery than that was seen in the CKO^{Dnmt3a+/-} mice above. This corresponds to the two week-time period that is required for AAV to reach its peak/plateau of gene expression after intravitreal injection (not shown). In contrast, the control mice received injection of AAV-scrambled RNA showed no positive OMR up to 12 weeks after injury. By 12 weeks post-ONC, AAV-shRNA-treated mice regained light perception and exhibited no difference in light/dark box assays compared to their uninjured baselines (FIG. 5C). Moreover, the AAV-shRNA-treated mice demonstrated improved pSTR and N1 amplitude of VEP compared to the control mice that received AAV-scrambled RNA injection, which remained blind without any sign of light perception or VEP response (FIG. 5D-F). Similar recovery of visual acuity, light perception, pSTR, and VEP improvement were also observed in fl/− mice that received AAV-Cre compared to the AAV-GFP injected mice (FIG. 5G-M). Similarly, delays in VEP N1 latency were observed in both the AAV-shRNA-treated wildtype mice and the AAV-Cre-injected fl/+ mice, suggesting impaired myelination. These results demonstrate that Dnmt3a inhibition holds an immense therapeutic potential for the treatment of optic nerve diseases or injury, offering a viable avenue for restoring vision in vivo.

### REFERENCES

1 Varadarajan, S. G., Hunyara, J. L., Hamilton, N. R., Kolodkin, A. L. & Huberman, A. D. Central nervous system regeneration. Cell 185, 77-94 (2022). doi.org:10.1016/j.cell.2021.10.029
2 Williams, P. R., Benowitz, L. I., Goldberg, J. L. & He, Z. Axon Regeneration in the Mammalian Optic Nerve. Annu Rev Vis Sci 6, 195-213 (2020). doi.org:10.1146/annurev-vision-022720-094953
3 Kristensen, L. S., Nielsen, H. M. & Hansen, L. L. Epigenetics and cancer treatment. Eur J Pharmacol 625, 131-142 (2009). doi.org:10.1016/j.ejphar.2009.10.011
4 Chen, D. F. & Tonegawa, S. Why do mature CNS neurons of mammals fail to re-establish connections following injury—functions of bcl-2. Cell Death Differ 5, 816-822 (1998). doi.org:10.1038/sj.cdd.4400431
5 Chen, D. F., Schneider, G. E., Martinou, J. C. & Tonegawa, S. Bcl-2 promotes regeneration of severed axons in mammalian CNS. Nature 385, 434-439 (1997).
6 Chen. D. F., Jhaveri. S. & Schneider. G. E. Intrinsic changes in developing retinal neurons result in regenerative failure of their axons. Proc Natl Acad Sci USA 92, 7287-7291 (1995). doi.org:10.1073/pnas.92.16.7287
7 Goldberg, J. L., Klassen, M. P., Hua, Y. & Barres, B. A. Amacrine-signaled loss of intrinsic axon growth ability by retinal ganglion cells. Science 296, 1860-1864 (2002). doi.org: 10.1126/science.1068428
8 Rao, R. C. et al. Dynamic patterns of histone lysine methylation in the developing retina. Invest Ophthalmol Vis Sci 51, 6784-6792 (2010). doi.org:10.1167/iovs.09-4730
9 Lu, Y. et al. Reprogramming to recover youthful epigenetic information and restore vision. Nature 588, 124-129 (2020). doi.org:10.1038/s41586-020-2975-4
10 Juarez-Mercado, K. E. et al. Expanding the Structural Diversity of DNA Methyltransferase Inhibitors. Pharmaceuticals (Basel) 14 (2020). doi.org:10.3390/ph14010017
11 Day, J. J. & Sweatt, J. D. DNA methylation and memory formation. Nat Neurosci 13, 1319-1323 (2010). doi.org:10.1038/nn.2666
12 Ji, Y., Zhao, M., Qiao, X. & Peng, G. H. Decitabine improves MMS-induced retinal photoreceptor cell damage by targeting DNMT3A and DNMT3B. Front Mol Neurosci 15, 1057365 (2022). doi.org:10.3389/fnmol.2022.1057365
13 Farinelli, P. et al. DNA methylation and differential gene regulation in photoreceptor cell death. Cell Death Dis 5, e1558 (2014). doi.org:10.1038/cddis.2014.512
14 Chen, Z. & Zhang, Y. Role of Mammalian DNA Methyltransferases in Development. Annu Rev Biochem 89, 135-158 (2020). doi.org:10.1146/annurev-biochem-103019-102815
15 Jackson-Grusby, L. et al. Loss of genomic methylation causes p53-dependent apoptosis and epigenetic deregulation. Nat Genet 27, 31-39 (2001). doi.org:10.1038/83730
16 Kaneda, M. et al. Essential role for de novo DNA methyltransferase Dnmt3a in paternal and maternal imprinting. Nature 429, 900-903 (2004). doi.org:10.1038/nature02633
17 Vong, L. et al. Leptin action on GABAergic neurons prevents obesity and reduces inhibitory tone to POMC neurons. Neuron 71, 142-154 (2011). doi.org:10.1016/j.neuron.2011.05.028
18 Tran, N. M. et al. Single-Cell Profiles of Retinal Ganglion Cells Differing in Resilience to Injury Reveal Neuroprotective Genes. Neuron 104, 1039-1055 e1012 (2019). doi.org:10.1016/j.neuron.2019.11.006
19 Wassle. H., Regus-Leidig. H. & Haverkamp, S. Expression of the vesicular glutamate transporter vGluT2 in a subset of cones of the mouse retina. J Comp Neurol 496, 544-555 (2006). doi.org:10.1002/cne.20942
20 Bamstable, C. J. & Drager, U. C. Thy-1 antigen: a ganglion cell specific marker in rodent retina. Neuroscience 11, 847-855 (1984). doi.org:10.1016/0306-4522(84)90195-7
21 Perry, V. H., Morris, R. J. & Raisman, G. Is Thy-1 expressed only by ganglion cells and their axons in the retina and optic nerve? J Neurocytol 13, 809-824 (1984). doi.org:10.1007/BF01148495
22 Cho, K. S. et al. Re-establishing the regenerative potential of central nervous system axons in postnatal mice. J Cell Sci 118, 863-872 (2005). doi.org:10.1242/jcs.01658
23 Pernet, V. & Schwab, M. E. Lost in the jungle: new hurdles for optic nerve axon regeneration. Trends Neurosci 37, 381-387 (2014). doi.org:10.1016/j.tins.2014.05.002
24 Conceicao, R. et al. Expression of Developmentally Important Axon Guidance Cues in the Adult Optic Chiasm. Invest Ophthalmol Vis Sci 60, 4727-4739 (2019). doi.org:10.1167/iovs.19-26732
25 Shi. C. et al. Optimization of Optomotor Response-based Visual Function Assessment in Mice. Sci Rep 8, 9708 (2018). doi.org:10.1038/s41598-018-27329-w
26 Ridder, W. H., 3rd & Nusinowitz, S. The visual evoked potential in the mouse—origins and response characteristics. Vision Res 46, 902-913 (2006). doi.org:10.1016/j.visres.2005.09.006
27 You, Y., Klistorner, A. Thie. J. & Graham. S. L. Latency delay of visual evoked potential is a real measurement of demyelination in a rat model of optic neuritis. Invest Ophthalmol Vis Sci 52, 6911-6918 (2011). doi.org:10.1167/iovs.11-7434
28 Tang. Y. et al. Therapeutic Targeting of Retinal Immune Microenvironment With CSF-1 Receptor Antibody Promotes Visual Function Recovery After Ischemic Optic Neuropathy. Front Immunol 11, 585918 (2020). doi.org:10.3389/fimmu.2020.585918
29 Jacobi, A. et al. Overlapping transcriptional programs promote survival and axonal regeneration of injured retinal ganglion cells. Neuron 110, 2625-2645 e2627 (2022). doi.org:10.1016/j.neuron.2022.06.002
30 Hahn, J. et al. Evolution of neuronal cell classes and types in the vertebrate retina. bioRxiv (2023). doi.org:10.1101/2023.04.07.536039
31 Lim, J. H. et al. Neural activity promotes long-distance, target-specific regeneration of adult retinal axons. Nat Neurosci 19, 1073-1084 (2016). doi.org:10.1038/nn.4340
32 Duan, X. et al. Subtype-specific regeneration of retinal ganglion cells following axotomy: effects of osteopontin and mTOR signaling. Neuron 85, 1244-1256 (2015). doi.org:10.1016/j.neuron.2015.02.017
33 Rheaume, B. A. et al. Pten inhibition dedifferentiates long-distance axon-regenerating intrinsically photosensitive retinal ganglion cells and upregulates mitochondria-associated Dynlt1a and Lars2. Development 150 (2023). doi.org:10.1242/dev.201644
34 Sun, F. et al. Sustained axon regeneration induced by co-deletion of PTEN and SOCS3. Nature 480, 372-375 (2011). doi.org:10.1038/nature10594
35 Jin. S. et al. Inference and analysis of cell-cell communication using CellChat. Nat Commun 12, 1088 (2021). doi.org:10.1038/s41467-021-21246-9
36 Park, B. et al. Granulin is a soluble cofactor for toll-like receptor 9 signaling. Immunity 34, 505-513 (2011). doi.org:10.1016/j.immuni.2011.01.018
37 Rao, K. & Lund, R. D. Optic nerve degeneration induces the expression of MHC antigens in the rat visual system. J Comp Neurol 336. 613-627 (1993). doi.org:10.1002/cne.903360413
38 Carulli. D. de Winter, F. & Verhaagen, J. Semaphorins in Adult Nervous System Plasticity and Disease. Front Synaptic Neurosci 13, 672891 (2021). doi.org:10.3389/fnsyn.2021.672891
39 Zhou, H. et al. Angiopoietin-2 induces the neuronal differentiation of mouse embryonic NSCs via phosphatidylinositol 3 kinase-Akt pathway-mediated phosphorylation of mTOR. Am J Transl Res 11, 1895-1907 (2019).
40 Luck, R. et al. The angiopoietin-Tie2 pathway regulates Purkinje cell dendritic morphogenesis in a cell-autonomous manner. Cell Rep 36, 109522 (2021). doi.org:10.1016/j.celrep.2021.109522
41 Li, S. & Jakobs, T. C. Secreted phosphoprotein 1 slows neurodegeneration and rescues visual function in mouse models of aging and glaucoma. Cell Rep 41, 111880 (2022). doi.org:10.1016/j.celrep.2022.111880
42 Garcia, A. L., Udeh, A., Kalahasty, K. & Hackam, A. S. A growing field: The regulation of axonal regeneration by Wnt signaling. Neural Regen Res 13, 43-52 (2018). doi.org:10.4103/1673-5374.224359
43 Musada, G. R., Carmy-Bennun, T. & Hackam, A. S. Identification of a Novel Axon Regeneration Role for Noncanonical Wnt Signaling in the Adult Retina after Injury. eNeuro 9 (2022). doi.org:10.1523/ENEURO.0182-22.2022
44 Johnson, T. V. et al. Identification of retinal ganglion cell neuroprotection conferred by platelet-derived growth factor through analysis of the mesenchymal stem cell secretome. Brain 137, 503-519 (2014). doi.org:10.1093/brain/awt292
45 Ikeda, W. et al. Nectin-like molecule-5/Tage4 enhances cell migration in an integrin-dependent, Nectin-3-independent manner. J Biol Chem 279, 18015-18025 (2004). doi.org:10.1074/jbc.M312969200
46 Kajita, M., Ikeda, W., Tamaru. Y. & Takai, Y. Regulation of platelet-derived growth factor-induced Ras signaling by poliovirus receptor Necl-5 and negative growth regulator Sprouty2. Genes Cells 12, 345-357 (2007). doi.org:10.1111/j.1365-2443.2007.01062.x
47 Erfani, S. et al. Nampt/PBEF/visfatin exerts neuroprotective effects against ischemia/reperfusion injury via modulation of Bax/Bcl-2 ratio and prevention of caspase-3 activation. J Mol Neurosci 56, 237-243 (2015). doi.org:10.1007/s12031-014-0486-1
48 Erfani, S. et al. Visfatin reduces hippocampal CA1 cells death and improves learning and memory deficits after transient global ischemia/reperfusion. Neuropeptides 49, 63-68 (2015). doi.org:10.1016/j.npep.2014.12.004
49 Carmeliet, P. Mechanisms of angiogenesis and arteriogenesis. Nat Med 6, 389-395 (2000). doi.org:10.1038/74651
50 Daies, J. M., Schellhardt, L. & Wood, M. D. The Role of the IL-4 Signaling Pathway in Traumatic Nerve Injuries. Neurorehabil Neural Repair 35, 431-443 (2021). doi.org:10.1177/15459683211001026
51 Milner, J. D. et al. Sustained IL-4 exposure leads to a novel pathway for hemophagocytosis, inflammation, and tissue macrophage accumulation. Blood 116, 2476-2483 (2010). doi.org:10.1182/blood-2009-11-255174
52 Rakyan, V. K., Down, T. A., Balding, D. J. & Beck, S. Epigenome-wide association studies for common human diseases. Nat Rev Genet 12, 529-541 (2011). doi.org:10.1038/nrg3000
53 Patel, A. K., Park, K. K. & Hackam, A. S. Wnt signaling promotes axonal regeneration following optic nerve injury in the mouse. Neuroscience 343, 372-383 (2017). doi.org:10.1016/j.neuroscience.2016.12.020
54 Plachez, C. et al. Robos are required for the correct targeting of retinal ganglion cell axons in the visual pathway of the brain. Mol Cell Neurosci 37, 719-730 (2008). doi.org:10.1016/j.mcn.2007.12.017
55 Sudhof, T. C. Neuroligins and neurexins link synaptic function to cognitive disease. Nature 455, 903-911 (2008). doi.org:10.1038/nature07456
56 Yang, Y. & Calakos, N. Muncl3-1 is required for presynaptic long-term potentiation. J Neurosci 31, 12053-12057 (2011). doi.org:10.1523/JNEUROSCL.2276-11.2011
57 Levkovitch-Verbin, H. Retinal ganglion cell apoptotic pathway in glaucoma: Initiating and downstream mechanisms. Prog Brain Res 220, 37-57 (2015). doi.org:10.1016/bs.pbr.2015.05.005
58 Han, X. et al. Gas5 inhibition promotes the axon regeneration in the adult mammalian nervous system. Exp Neurol 356, 114157 (2022). doi.org:10.1016/j.expneurol.2022.114157

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects. advantages, and modifications are within the scope of the following claims.

What is claimed is:

1. A method of restoring vision in a subject with an optic nerve injury or condition, the method comprising administering to the eye of the subject a therapeutically effective amount of an DNMT3a inhibitor.

2. The method of claim 1, wherein the DNMT3a inhibitor is a small molecule inhibitor selected from the group consisting of: decitabine, SAHA, GSKex1, compound 40, compound 40_3, Compound 15a, SGI-1027, MC3343, MC3353, BIX-01294, CM-272, CM-579, UVI5008, and propiophenone.

3. The method of claim 1, wherein the DNMT3a inhibitor is an inhibitory nucleic acid that binds to a portion of DNMT3A.

4. The method of claim 3, wherein the inhibitory nucleic acid is antisense RNA, antisense DNA, chimeric antisense oligonucleotides, interference RNA (RNAi), short interfering RNA (siRNA); or a short, hairpin RNA (shRNA).

5. The method of claim 1, wherein administering to the eye of the subject an AAV vector comprises the inhibitory nucleic acid that binds to DNMT3A.

6. The method of claim 5, wherein the AAV vector comprises AAV2.

7. The method of claim 5, wherein the administration comprises intravitreal injection.

8. The method of claim 1, wherein the optic nerve injury or condition is a physical injury.

9. The method of claim 8, wherein the physical injury comprises compression injury, ischemia, stroke, trauma, shear force injury, tear injury, and/or surgery.

10. The method of claim 1, wherein the optic nerve injury or condition comprises glaucoma, optic neuropathy, ischemic optic neuropathy, optic neuritis, optic nerve atrophy, idiopathic intracranial hypertension, or whole eye transplantation.

11. The method of claim 1, wherein the subject is an adult.

12. A nucleic acid having the sequence of SEQ ID NO: 2 or SEQ ID NO: 3.

13. A vector encoding a nucleic acid having the sequence of SEQ ID NO: 2 or SEQ ID NO: 3.


SEQUENCE LISTING (3 sequences)

SEQ ID NO: 1 - RNA, 48 nt, synthetic construct
  source 1..48: mol_type = other RNA
cctaaggttaagtcgccctcgctcgagcgagggcgacttaaccttagg

SEQ ID NO: 2 - RNA, 48 nt, synthetic construct
  source 1..48: mol_type = other RNA
cgctccgctgaaggaatatttctcgagaaatattccttcagcggagcg

SEQ ID NO: 3 - RNA, 48 nt, synthetic construct
  source 1..48: mol_type = other RNA
ggcatccactgtgaatgataactcgagttatcattcacagtggatgcc